SEQUENCE LISTING (3 sequences)

SEQ ID NO: 1 - DNA, 20 bp, Unknown
  Description of Unknown Nucleating polypeptide binding motif sequence
  modified_base (1)..(1): a, c, t or g
  modified_base (3)..(3): a, c, t or g
nbndccdshagrkgghrshv

SEQ ID NO: 2 - DNA, 20 bp, Unknown
  Description of Unknown Nucleating polypeptide binding motif sequence
  modified_base (18)..(18): a, c, t or g
  modified_base (20)..(20): a, c, t or g
vhsrhggkrgahsdccdnbn

SEQ ID NO: 3 - DNA, 7 bp, Artificial Sequence
  Description of Artificial Sequence Synthetic oligonucleotide
tatawaw

(19) United States
(12) Patent Application Publication
(10) Pub. No.: US 2022/0267756 A1
(43) Pub. Date: Aug. 25, 2022

(54) COMPOSITIONS AND METHODS FOR MODULATING GENOMIC COMPLEX INTEGRITY INDEX

(71) Applicant: Flagship Pioneering Innovations V, Inc., Cambridge, MA (US)

(72) Inventors: Laura Gabriela Lande, Chestnut Hill, MA (US); David Arthur Berry, Newton, MA (US); Rahul Karnik, Cambridge, MA (US)

(21) Appl. No.: 17/754,050

(22) PCT Filed: Sep. 22, 2020
(86) PCT No.: PCT/US2020/051988

§ 371 Date: Mar. 22, 2022

Related U.S. Application Data
(60) Provisional application No. 62/904,310, filed on Sep. 23, 2019.

(51) Int. Cl. C12N 15/10; C12N 15/63; C12N 9/22; G16B 20/40

(52) U.S. Cl. CPC C12N 15/102; G16B 20/40; C12N 9/22; C12N 15/63

(57) ABSTRACT

The present disclosure relates generally to modulation of genomic complexes via modulation (e.g., disruption) based on certain integrity index scores.

### CROSS-REFERENCE TO RELATED APPLICATIONS

The present application claims priority to and benefit from U.S. provisional application U.S. Ser. No. 62/904,310 (filed Sep. 23, 2019), the contents of which is herein incorporated by reference.

### BACKGROUND

Certain genomic structures can affect gene expression. In targeting genomic structures for modulation to affect gene expression, it can be helpful to understand characteristics of the genomic structures, e.g., how frequently they occur and in which types of cells. There is a need for methods and compositions that evaluate genomic structures and apply said evaluations to better affect gene expression.

### SUMMARY

The present disclosure provides, in part, technologies and methods for modulating (e.g., disrupting) a genomic complex, e.g., anchor sequence-mediated conjunctions (ASMC), in a subject (e.g., a mammalian subject) by administering a modulating agent (e.g., disrupting agent) targeted to the genomic complex (e.g., ASMC) to the subject, wherein the genomic complex (e.g., ASMC) has or has been identified as having an integrity index (e.g., as measured by Formula 2 or 3 described herein) of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0). Those skilled in the art are familiar with genomic complexes, including those comprising anchor sequence-mediated conjunctions (e.g., genomic loops). Modulation, e.g., disruption, of a genomic complex (e.g., ASMC) can affect, for example, expression of target genes associated with said genomic complex (e.g., ASMC). The integrity index of a genomic complex (e.g., ASMC) is, in part, a value representing the frequency of incidence of the genomic complex (e.g., ASMC) in a given cell population and optionally in a given time period.

Without wishing to be bound by theory, it is thought that modulating, e.g., disrupting, a genomic complex (e.g., ASMC) having or having been identified as having a high integrity index (e.g., 0.5-1) may have an improved (e.g., increased) effect, e.g., on expression of a target gene associated with the genomic complex, relative to modulation of a genomic complex without regard to integrity index or modulation of a genomic complex having a low integrity index (e.g., less than 0.5 (and optionally greater than 0)). A genomic complex having a higher integrity index indicates the genomic complex is present more frequently in a given cell population and/or time period, and/or the genomic sequence elements of the genomic complex are more strongly associated with one another. Without wishing to be bound by theory, modulation, e.g., disruption, of such a genomic complex may have a more significant effect on expression of an associated target gene than a similar modulation of a more weakly or infrequently associated genomic complex.

Without wishing to be bound by theory, it is thought that modulating, e.g., disrupting, a genomic complex (e.g., ASMC) having or having been identified as having an intermediate integrity index (e.g., 0.25-0.75) may have an improved (e.g., increased) effect, e.g., on expression of a target gene associated with the genomic complex, relative to modulation of a genomic complex without regard to integrity index, modulation of a genomic complex having a low integrity index (e.g., less than 0.25 (and optionally greater than 0)), or modulation of a genomic complex having a high integrity index (e.g., greater than 0.75 (and optionally less than or equal to 1)). A genomic complex having an intermediate integrity index indicates the genomic complex is dynamically present and absent in a given cell population and/or time period, and/or the genomic sequence elements of the genomic complex are strongly associated enough to interact frequently but weakly associated enough to disengage with one another frequently too. Without wishing to be bound by theory, modulation, e.g., disruption, of such a genomic complex may have a more significant effect on expression of an associated target gene than a similar modulation of a more weakly or infrequently associated genomic complex or a stronger more frequently associated genomic complex. A modulating agent, e.g., disrupting agent, described herein may be more likely to achieve modulation, e.g., disruption, of a genomic complex (e.g., ASMC) having or having been identified as having an intermediate integrity index due to the malleable, dynamic interaction(s) maintaining/forming the genomic complex.

The present disclosure also provides, in part, technologies for modulating (e.g., disrupting) a genomic complex, e.g., anchor sequence-mediated conjunctions (ASMC), in a subject (e.g., a mammalian subject) by administering a modulating agent (e.g., disrupting agent) targeted to the genomic complex (e.g., ASMC) to the subject, wherein the genomic complex (e.g., ASMC) has or has been identified as having a specificity index (e.g., as measured by Formula 1 or the methods of Example 1) that is less than a threshold value (e.g., a specificity index less than 0.5). The specificity index of a genomic complex (e.g., ASMC) is, in part, a value representing the rarity of a genomic complex (e.g., ASMC) across a plurality of cell populations. Without wishing to be bound by theory, it may be advantageous to target a genomic complex (e.g., ASMC) that is present in a target cell or cell type of interest and that has a low specificity index (e.g., less than 0.5). A low specificity index indicates that a genomic complex (e.g., ASMC) is present in fewer cell populations than a genomic complex having a high specificity index. Targeting a genomic complex (e.g., ASMC) with a low specificity index may cause fewer off-target effects in non-target cells by virtue of the target genomic complex not being present in as many non-target cells. For example, it may be advantageous to target a genomic complex (e.g., ASMC) present only in a cell type of interest for the purposes of altering expression of a target gene associated with the target genomic complex, because it is less likely (e.g., not likely) that targeting said genomic complex would affect expression of the target gene in other cell types not comprising the target genomic complex.

The present disclosure also provides modulating agents, e.g., disrupting agents, for use in the methods described herein. In some embodiments, a modulating agent, e.g., disrupting agent, binds a genomic complex (e.g., ASMC), wherein the genomic complex (e.g., ASMC) has or has been identified as having an integrity index (e.g., as measured by Formula 2 or 3 described herein) of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0). In some embodiments, a modulating agent, e.g., disrupting agent, binds a genomic complex (e.g., ASMC), wherein the genomic complex (e.g., ASMC) has or has been identified as having a specificity index of less than 0.5 (e.g., less than 0.45, 0.4, 0.35, 0.3, 0.25, 0.2, 0.15, 0.1, or 0.05).

The present disclosure also provides, in part, technologies and methods for selecting a subject (e.g., a mammalian subject, e.g., a human subject) for administration of a modulating agent (e.g., a disrupting agent) to modulate (e.g., disrupt) a genomic complex, e.g., anchor sequence-mediated conjunctions (ASMC), comprising identifying a value for the integrity index (e.g., as measured by Formula 2 or 3) of the genomic complex (e.g., ASMC) in the subject, and, if the integrity index is within a predetermined range (e.g., between 0.25-0.75 (e.g., 0.3-0.4, 0.4-0.5, 0.5-0.6, or 0.6-0.7), or between 0.5-1 (e.g., about 0.5-0.6, 0.6-0.7, 0.7-0.8, 0.8-0.9, or 0.9-1.0)), then selecting the subject for administration of the modulating agent, e.g., disrupting agent. Without wishing to be bound by theory, it may be advantageous to administer a modulating agent (e.g., disrupting agent) to a subject having a target genomic complex (e.g., ASMC) that has or has been identified as having an integrity index within a predetermined range as compared to a subject not having a target genomic complex (e.g., ASMC) that has or has been identified as having an integrity index within a predetermined range. For example, a subject having a target cell type comprising a target genomic complex having an intermediate integrity index may, due to the malleable, dynamic interaction(s) maintaining/forming said genomic complex, achieve a more effective (e.g., increased) modulation (e.g., disruption) of said genomic complex upon being administered a modulating agent (e.g., disrupting agent) than a subject not having a target genomic complex having an intermediate integrity index or a subject having a target genomic complex having an integrity index that is not intermediate (e.g., a high or low integrity index). As a further example, a subject having a target cell type comprising a target genomic complex having a high integrity index may, due to the strength of the interactions maintaining/forming said genomic complex and/or the frequency of the incidence of said genomic complex, achieve a more effective (e.g., increased) modulation (e.g., disruption) of said genomic complex upon being administered a modulating agent (e.g., disrupting agent) than a subject not having a target genomic complex having a high integrity index or a subject having a target genomic complex having an integrity index that is not high (e.g., is low).

The present disclosure also provides, in part, technologies and methods for selecting a subject (e.g., a mammalian subject, e.g., a human subject) for administration of a modulating agent (e.g., a disrupting agent) to modulate (e.g., disrupt) a genomic complex, e.g., anchor sequence-mediated conjunctions (ASMC), comprising determining whether the genomic complex (e.g., ASMC) is present in a target cell type and/or one or more non-target cell types in the subject, and, if the genomic complex (e.g., ASMC) is not present in at least one non-target cell type in the subject, then selecting the subject for administration of the modulating agent (e.g., disrupting agent). In some embodiments, determining comprises identifying a value for the specificity index (e.g., as measured by Formula 1) of the genomic complex (e.g., ASMC) in the subject, and, if the specificity index is less than a threshold value (e.g., a specificity index less than 1, e.g., less than 0.5), then selecting the subject for administration of the modulating agent, e.g., disrupting agent.

Without wishing to be bound by theory, it may be advantageous to administer a modulating agent (e.g., disrupting agent) to a subject having a target genomic complex (e.g., ASMC) that is present in a target cell type, wherein: (i) the subject has or has been identified as having at least one non-target cell type in which the genomic complex (e.g., ASMC) is not present as compared to a subject that has or has been identified as having fewer (e.g., no) non-target cell types in which the genomic complex (e.g., ASMC) is not present; or (ii) the target genomic complex (e.g., ASMC) has or has been identified as having a specificity index less than a threshold value (e.g., as compared to a subject not having a target genomic complex (e.g., ASMC) that has or has been identified as having a specificity index less than a threshold value or a subject having a target genomic complex (e.g., ASMC) that has or has been identified as having a specificity index at or above the threshold value). For example, a subject having at least one non-target cell type in which the genomic complex (e.g., ASMC) is not present may, due to the lower incidence of the target genomic complex in non-target cells/tissues, experience fewer side effects and/or off-target genomic complex modulation upon being administered a modulating agent (e.g., disrupting agent) than a subject in which the genomic complex (e.g., ASMC) is present in more, e.g., all, non-target cell types. As a further example, a subject having a target genomic complex having a low specificity index may, due to the lower incidence of the target genomic complex in non-target cells/tissues, experience fewer side effects and/or off-target genomic complex modulation upon being administered a modulating agent (e.g., disrupting agent) than a subject not having a target genomic complex having a low specificity index (e.g., a high specificity index).

The present disclosure also provides, in part, technologies and methods for evaluating a genomic complex (e.g., ASMC) in a target cell, comprising, determining whether the genomic complex (e.g, ASMC) is present in the target cell, and determining whether the genomic complex (e.g., ASMC) is present in one or more non-target cells, e.g., one or more reference cell types, e.g., one or more (e.g., all) reference cell types of Table 2. In some embodiments, a method of evaluating a genomic complex (e.g., ASMC) in a target cell, comprises determining the specificity index for the genomic complex (e.g., ASMC) in a target cell, e.g., in relation to one or more reference cell types, e.g., one or more (e.g., all) reference cell types of Table 2. Without wishing to be bound by theory, understanding whether a genomic complex (e.g., ASMC) of interest is present in a target cell of a subject and whether said complex is present in non-target cells of the subject is important for determining whether to administer a modulating agent (e.g., disrupting agent) to a subject. If the genomic complex (e.g., ASMC) is not present in the target cell of the subject, a modulating agent (e.g., disrupting agent) may not have any effect on expression, e.g., of a target gene associated with the genomic complex (e.g., ASMC). If the genomic complex (e.g., ASMC) is present in one or more non-target cell types of the subject, a modulating agent (e.g., disrupting agent) may have off-target effects (e.g., on expression of a target gene associated with the genomic complex (e.g., ASMC) in non-target cell types) and/or cause side effects for the subject.

The present disclosure also provides, in part, technologies and methods for evaluating a test modulating agent (e.g., a test disrupting agent) comprising contacting a test cell with the test modulating agent (e.g., a test disrupting agent), identifying in a genomic complex (e.g., ASMC) of interest in the test cell an integrity index (e.g., as measured by Formula 2 or Formula 3, e.g., by the methods of Example 2) of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0), and comparing the integrity index to a reference value (e.g., the integrity index of the genomic complex (e.g., ASMC) in a control cell, e.g., a control cell that is otherwise similar to the test cell but that was not contacted with the test modulating agent (e.g., disrupting agent)). Without wishing to be bound by theory, it is thought that if a test modulating agent (e.g., disrupting agent) is a modulating agent (e.g., disrupting agent), the integrity index of the genomic complex (e.g., ASMC) of interest will be lower in cells contacted with the test modulating agent (e.g., disrupting agent) than in a control cell that is otherwise similar to the test cell but that was not contacted with the test modulating agent (e.g., disrupting agent).

The present disclosure also provides, in part, technologies and methods for evaluating a test modulating agent (e.g., a test disrupting agent) comprising contacting a test cell with the test modulating agent (e.g., a test disrupting agent), determining whether a genomic complex (e.g., ASMC) is present in the test cell, and contacting one or more non-target cells, e.g., one or more reference cell types, e.g., one or more (e.g., all) reference cell types of Table 2, with the test modulating agent (e.g., test disrupting agent). In some embodiments, a method of evaluating a test modulating agent (e.g., a test disrupting agent) comprises determining the specificity index for the genomic complex (e.g., ASMC) before and/or after contact with the test modulating agent, e.g., in relation to one or more reference cell types, e.g., one or more (e.g., all) reference cell types of Table 2.

Additional features of any of the aforesaid methods or compositions include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Sep. 23, 2019. When one gene or protein references a plurality of sequence accession numbers, all of the sequence variants are encompassed.

### ENUMERATED EMBODIMENTS

1. A method of disrupting a genomic complex, e.g., anchor sequence mediated conjunction (ASMC), in a mammalian subject, comprising:

administering to a subject a disrupting agent targeted to the genomic complex, e.g., ASMC,

wherein the genomic complex, e.g., ASMC, has, or is identified as having, an IntIndᵢ, measured by Formula 2

$$( {IntInd}_{i} = min ( \frac{Frequency of genomic complex \\ ( e . g . , ASMC ) i in cell sample}{95 th percentile frequency of all genomic \\ complexes ( e . g . , ASMCs ) within cell sample} , 1 ) ) ,$$

of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0).
2. A method of disrupting a genomic complex, e.g., anchor sequence mediated conjunction (ASMC), in a mammalian subject, comprising:

administering to a subject a disrupting agent targeted to the genomic complex, e.g., ASMC, wherein the genomic complex, e.g., ASMC, has, or is identified as having, an IntInch, measured by Formula 3

$$( {IntInd}_{i} = min ( \frac{\begin{matrix}log 2 ( number of PETs supporting \\ genomic complex ( e . g . , ASMC ) i )\end{matrix}}{Normilzation factor} , 1 ) ) ,$$

of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0),

where the normalization factor is the 99th percentile of the base-2 logarithm of the number of PETs (paired end tags) supporting any single loop.

3. A method of disrupting a genomic complex, e.g., anchor sequence mediated conjunction (ASMC), in a mammalian subject, comprising:

administering to a subject a disrupting agent targeted to the genomic complex, e.g., ASMC,

wherein the genomic complex, e.g., ASMC, is present in a target cell type, and

wherein the genomic complex, e.g., ASMC, is present in less than 9, 8, 7, 6, 5, 4, 3, 2, or 1 reference cell types of Table 2.

4. The method of embodiment 3, wherein the target cell type is chosen from: neuronal cells (e.g., CNS cells), myocytes (e.g., cardiomyocytes), blood cells (e.g., immune cells), endothelial cells, hepatocytes, CD34+ cells, CD3+ cells, and fibroblasts.
5. A disrupting agent that specifically binds a genomic complex, e.g., ASMC,

wherein the genomic complex, e.g., ASMC, has, or is identified as having, an IntIndᵢ, measured by Formula 2

$$( {IntInd}_{i} = \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} ) ,$$

of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0).
6. A disrupting agent that specifically binds a genomic complex, e.g., ASMC,

wherein the genomic complex, e.g., ASMC, has, or is identified as having, an IntIndᵢ,

measured by Formula 3

$$( {IntInd}_{i} = min ( \frac{\begin{matrix}log 2 ( number of PETs supporting \\ genomic complex ( e . g . , ASMC ) i )\end{matrix}}{Normalization factor} , 1 ) ) ,$$

of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0).
7. A disrupting agent that specifically binds a genomic complex, e.g., ASMC,

wherein the genomic complex, e.g., ASMC, is present in a target cell type, and

wherein the genomic complex, e.g., ASMC, is present in less than 9, 8, 7, 6, 5, 4, 3, 2, or 1 reference cell types of Table 2.

8. The disrupting agent of any of embodiments 5-7, wherein the disrupting agent comprises a nucleic acid complementary to DNA sequence of the genomic complex, e.g., ASMC.
9. A method of selecting a mammalian subject for administration of a disrupting agent to disrupt a genomic complex, e.g., anchor sequence mediated conjunction (ASMC), comprising:

identifying a value of an IntIndᵢ, measured by Formula 2

$$( {IntInd}_{i} = min ( \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} , 1 ) ) ,$$

of the ASMC in the mammalian subject, and

if the IntIndᵢ is within a predetermined range, then selecting the subject for administration of the disrupting agent.

10. A method of selecting a mammalian subject for administration of a disrupting agent to disrupt a genomic complex, e.g., anchor sequence mediated conjunction (ASMC), comprising:

identifying a value of an IntIndᵢ, measured by Formula 3

$$( {IntInd}_{i} = min ( \frac{\begin{matrix}log 2 ( number of PETs supporting \\ genomic complex ( e . g . , ASMC ) i )\end{matrix}}{Normalization factor} , 1 ) ) ,$$

of the genomic complex, e.g., ASMC, in the mammalian subject, and

if the IntIndᵢ is within a predetermined range, then selecting the subject for administration of the disrupting agent.

11. The method of embodiment 9 or 10, which further comprises administering to the mammalian subject a disrupting agent targeted to the genomic complex, e.g., ASMC.
12. The method of any of embodiments 9-11, wherein the value of the IntIndᵢ as measured by Formula 2 or Formula 3 is, or the predetermined range is, between 0.25-0.75 (e.g., 0.3-0.4, 0.4-0.5, 0.5-0.6, or 0.6-0.7), or between 0.5-1 (e.g., about 0.5-0.6, 0.6-0.7, 0.7-0.8, 0.8-0.9, or 0.9-1.0).
13. A method of selecting a mammalian subject for administration of a disrupting agent to disrupt a genomic complex, e.g., anchor sequence mediated conjunction (ASMC), comprising:

determining whether the genomic complex, e.g., ASMC, is present in a target cell type in the subject, and

determining whether the genomic complex, e.g., ASMC, is present in one or more non-target cell types in the subject

and, if the genomic complex, e.g., ASMC, is not present in at least one non-target cell type in the subject, then selecting the subject for administration of the disrupting agent.

14. A method of evaluating a genomic complex, e.g., anchor sequence mediated conjunction (ASMC), in a cell, comprising:

identifying, in the genomic complex, e.g., ASMC, in the cell, an IntIndᵢ, measured by Formula 2

$$( {IntInd}_{i} = min ( \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} , 1 ) ) ,$$

of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0).
15. A method of evaluating a genomic complex, e.g., anchor sequence mediated conjunction (ASMC), in a cell, comprising:

identifying, in the genomic complex, e.g., ASMC, in the cell, an IntIndᵢ, measured by Formula 3 of

$$( {IntInd}_{i} = min ( \frac{\begin{matrix}log 2 ( number of PETs supporting \\ genomic complex ( e . g . , ASMC ) i )\end{matrix}}{Normalization factor} , 1 ) ) ,$$

between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0).
16. The method of embodiment 14 or 15, which further comprises contacting the cell with a disrupting agent targeted to the genomic complex, e.g., ASMC.
17. The method of embodiment 14 or 15, which further comprises, after contacting the cell with the disrupting agent, an additional step of identifying, in the genomic complex, e.g., ASMC, in the cell, an IntIndᵢ as measured by Formula 2 or Formula 3.
18. A method of evaluating a genomic complex, e.g., anchor sequence mediated conjunction (ASMC), in a target cell, comprising:

determining whether the genomic complex, e.g., ASMC, is present in the target cell, and

determining whether the genomic complex, e.g., ASMC, is present in one or more non-target cell, e.g., one or more (e.g., all) reference cell types of Table 2.

19. A method of evaluating a test disrupting agent, comprising:

contacting a test cell with the test disrupting agent,

identifying, in a genomic complex, e.g., ASMC, in the test cell, an IntIndᵢ, measured by Formula 2

$$( {IntInd}_{i} = min ( \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} , 1 ) ) ,$$

of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0), and

comparing the IntIndᵢ to a reference value, e.g., wherein the reference value is the IntIndᵢ of the genomic complex, e.g., ASMC, in a control cell, e.g., wherein the control cell is an otherwise similar cell that was not contacted with the test disrupting agent.

20. A method of evaluating a test disrupting agent, comprising:

contacting a test cell with the test disrupting agent,

identifying, in a genomic complex, e.g., ASMC, in the cell, an IntIndᵢ, measured by Formula 3

$$( {IntInd}_{i} = min ( \frac{\begin{matrix}log 2 ( number of PETs supporting \\ genomic complex ( e . g . , ASMC ) i )\end{matrix}}{Normalization factor} , 1 ) ) ,$$

between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0), and

comparing the IntIndᵢ to a reference value, e.g., wherein the reference value is the IntIndᵢ of the genomic complex, e.g., ASMC, in a control cell, e.g., wherein the control cell is an otherwise similar cell that was not contacted with the test disrupting agent.

21. A method of evaluating a test disrupting agent, comprising:

contacting a test cell with the test disrupting agent,

determining whether a genomic complex, e.g., ASMC, is present in the test cell, and

contacting one or more (e.g., all) reference cell types (e.g., reference cell types of Table 2) with the test disrupting agent.

22. The method of any of embodiments 19-21, which comprises contacting each of a plurality of test cells with each of a plurality of test disrupting agents, e.g., from a library of compounds.
23. The method or composition of any of embodiments 1, 5, 9, 14, or 19, wherein the IntIndᵢ is measured using ChIA-PET, e.g., against CTCF, e.g., as described in Example 2.
24. The method of any of embodiments 2, 6, 10, 15, or 20, wherein the IntIndᵢ is measured using ChIA-PET, e.g., against CTCF, e.g., as described in Example 2.
25. The method of any of embodiments 3, 7, 13, 18, or 21 wherein genomic complex, e.g., ASMC, presence is measured by ChIA-PET, e.g., against cohesin, e.g., using an assay of Example 1.
26. The method of any of embodiments 1, 5, 9, 14, or 19, wherein the cell sample is a cell line sample or a primary cell sample (e.g., a biopsy sample).
27. The method of any of the preceding embodiments, wherein the disrupting agent comprises a DNA-binding moiety that binds specifically to one or more target anchor sequences within a cell and not to non-targeted anchor sequences within the cell with sufficient affinity that it competes with binding of an endogenous nucleating polypeptide within the cell.
28. The method of embodiment 27, wherein the disrupting agent further comprises a negative effector moiety associated with the DNA-binding moiety so that, when the DNA-binding moiety is bound at the one or more target anchor sequences, the negative effector moiety is localized thereto, the negative effector moiety being characterized in that dimerization of the endogenous nucleating polypeptide is reduced when the negative effector moiety is present as compared with when it is absent.
29. The method of any of the preceding embodiments, wherein the disrupting agent comprises (i) a site-specific targeting moiety and (ii) a deaminating agent.
30. The method of any of the preceding embodiments, wherein the disrupting agent comprises (i) a fusion polypeptide comprising an enzymatically inactive Cas polypeptide and a deaminating agent, or a nucleic acid encoding the fusion polypeptide; and (ii) a guide RNA, wherein the guide RNA targets the fusion polypeptide to an anchor sequence comprised by the genomic complex, e.g., ASMC.
31. The method of any of the preceding embodiments, wherein the disrupting agent comprises (i) a site-specific targeting moiety and (ii) an epigenetic modifying agent, e.g., wherein the epigenetic modifying agent is selected from a DNA methylase, DNA demethylase, histone methyltransferase, a histone deacetylase, or any combination thereof.
32. The method of any of the preceding embodiments, wherein the disrupting agent comprises (i) a fusion polypeptide comprising an enzymatically inactive Cas polypeptide and an epigenetic modifying agent, or a nucleic acid encoding the fusion polypeptide; and (ii) a guide RNA, wherein the guide RNA targets the fusion polypeptide to an anchor sequence comprised by the genomic complex, e.g., ASMC.
33. The method of any of the preceding embodiments, wherein the disrupting agent comprises a fusion polypeptide comprising a TAL effector molecule and an epigenetic modifying agent, or a nucleic acid encoding the fusion polypeptide, wherein the TAL effector molecule targets the fusion polypeptide to an anchor sequence comprised by the genomic complex, e.g., ASMC.
34. The method of any of the preceding embodiments, wherein the disrupting agent comprises a fusion polypeptide comprising a Zn finger molecule and an epigenetic modifying agent, or a nucleic acid encoding the fusion polypeptide, wherein the Zn finger molecule targets the fusion polypeptide to an anchor sequence comprised by the genomic complex, e.g., ASMC.
35. The method of any of the preceding embodiments, wherein the IntIndᵢ as measured by Formula 2 or Formula 3 in a cell of the subject, is reduced to less than 0.3-0.4, 0.4-0.5, 0.5-0.6, 0.7-0.8, or 0.8-0.9.
36. The method of any of the preceding embodiments, wherein the IntIndᵢ as measured by Formula 2 or Formula 3 in a cell of the subject, is reduced by at least 0.1, 0.2, 0.3, 0.4, 0.5, 0.6, 0.7. 0.8, or 0.9.
37. The method of any of the preceding embodiments, which further comprises, after administration of the disrupting agent, obtaining a value for (e.g., measuring) the IntIndᵢ as measured by Formula 2 or Formula 3 of the genomic complex, e.g., ASMC.
38. The method of embodiment 37, which further comprises, responsive to the value for the IntIndᵢ as measured by Formula 2 or Formula 3, administering one or more additional doses of the disrupting agent to the mammalian subject, or administering one or more different therapies.
39. The method of embodiment 38, which comprises administering the one or more additional doses of the disrupting agent to the mammalian subject until the IntIndᵢ as measured by Formula 2 or Formula 3 in a cell of the subject, is less than 0.9, 0.8, 0.7, 0.6, 0.5, 0.4, 0.3, 0.2, or 0.1.
40. The method of the preceding embodiments, which further comprises, after administration of the disrupting agent, determining obtaining a value for (e.g., measuring) expression of a gene associated with (e.g., situated at least partially within) the genomic complex, e.g., ASMC.
41. The method of embodiment 40, which further comprises, responsive to the value for the expression of the gene, administering one or more additional doses of the disrupting agent to the mammalian subject, or administering one or more different therapies.
42. The method of any of the preceding embodiments, wherein the genomic complex, e.g., ASMC, comprises a gene listed in Table 4 or 5.
43. The method of any of the preceding embodiments, wherein the genomic complex, e.g., ASMC, comprises an anchor sequence, or two anchor sequences, listed in Table 4 or 5.
44. The method of any of the preceding embodiments, wherein the genomic complex, e.g., ASMC, is bound by a polypeptide selected from CTCF, cohesin, YY1, USF1, TAF3, or ZNF143.
45. The method of any of the preceding embodiments wherein the genomic complex, e.g.,

ASMC, is a type 1 ASMC.

46. The method of any of embodiments 1-44, wherein the genomic complex, e.g., ASMC, is a type 2 ASMC.
47. The method of any of the preceding embodiments wherein disruption of the genomic complex, e.g., ASMC, results in upregulation of expression of a situated at least partly within the genomic complex, e.g., ASMC.
48. The method of any of embodiments 1-46, wherein disruption of the genomic complex, e.g., ASMC, results in downregulation of expression of a gene situated at least partly within the genomic complex, e.g., ASMC.
49. The method of embodiment 48, wherein the IntIndᵢ as measured by Formula 2 or Formula 3 of the genomic complex, e.g., ASMC, in the cell is at least 0.5, 0.55, 0.6, 0.65, 0.7, 0.75, 0.8, 0.85, or 0.9.

### Definitions

A, an, the: As used herein, the singular forms “a,” “an” and “the” include plural referents unless the context clearly dictates otherwise.
Agent: As used herein, the term “agent”, may be used to refer to a compound or entity of any chemical class including, for example, a polypeptide, nucleic acid, saccharide, lipid, small molecule, metal, or combination or complex thereof. As will be clear from context to those skilled in the art, in some embodiments, the term may be utilized to refer to an entity that is or comprises a cell or organism, or a fraction, extract, or component thereof. Alternatively or additionally, as those skilled in the art will understand in light of context, in some embodiments, the term may be used to refer to a natural product in that it is found in and/or is obtained from nature. In some embodiments, again as will be understood by those skilled in the art in light of context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some embodiments, the term “agent” may refer to a compound or entity that is or comprises a polymer; in some embodiments, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.
Anchor Sequence: The term “anchor sequence” as used herein, refers to a sequence recognized by a conjunction nucleating polypeptide (e.g., a nucleating polypeptide) that binds sufficiently to form an anchor sequence-mediated conjunction. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments of the present disclosure, technologies are provided that may specifically target a particular anchor sequence or anchor sequences, without targeting other anchor sequences (e.g., sequences that may contain a conjunction nucleating polypeptide (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated.
Anchor sequence-mediated conjunction: The term “anchor sequence-mediated conjunction” (also abbreviated ASMC) as used herein, refers to a DNA structure that occurs and/or is maintained via physical interaction or binding of at least two anchor sequences in the DNA by one or more proteins, such as nucleating polypeptides, or one or more proteins and/or a nucleic acid entity (such as RNA or DNA), that bind the anchor sequences to enable spatial proximity and functional linkage between the anchor sequences.
Associated with: Two events or entities are “associated” with one another, as that term is used herein, if presence, level, function, and/or form of one is correlated with that of the other. For example, in some embodiments, a particular entity (e.g., polypeptide, genetic signature, metabolite, microbe, etc.) is considered to be associated with a particular disease, disorder, or condition, if its presence, level, function, and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a target gene is “associated with” an anchor sequence-mediated conjunction if modulation (e.g., disruption) of the anchor sequence-mediated conjunction causes an alteration in expression (e.g., transcription) of the target gene. For example, in some embodiments, modulation (e.g., disruption) of an anchor sequence-mediated conjunction causes an enhancing or silencing/repressor sequence to associate with or become unassociated with a target gene, thereby altering expression of the target gene. In some embodiments, a target gene is associated with an ASMC if the target gene is situated within or partially within the ASMC.
Disruption: As will be understood by those skilled in the art, the term “disruption” is used to refer to a decrease in incidence (e.g., frequency, extent, etc) of a particular entity or event relating to an appropriate reference. For example, when used in reference to a particular genomic complex (e.g., a genomic complex at a particular genomic location or site), it means that incidence of that genomic complex at that genomic location or site is reduced relative to an appropriate reference (e.g., absence of a modulating agent as described herein). As will be appreciated by those skilled in the art, incidence may be reflected in presence (existence), formation, function, and/or stability of the relevant genomic complex (e.g., anchor sequence-mediated conjunction).
Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).
Genomic complex: As used herein, the term “genomic complex” is a complex that brings together two genomic sequence elements that are spaced apart from one another on one or more chromosomes, via interactions between and among a plurality of protein and/or other components (potentially including the genomic sequence elements). In some embodiments, the genomic sequence elements are anchor sequences to which one or more protein components of the complex binds. In some embodiments, a genomic complex may be an anchor sequence mediated conjunction (ASMC). In some embodiments, a genomic complex comprises one or more ASMCs. In some embodiments, a genomic sequence element may be or comprise an anchor sequence (e.g., a CTCF binding motif), a promoter and/or an enhancer. In some embodiments, a genomic sequence element includes at least one or both of a promoter and/or an enhancer. In some embodiments, genomic complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., enhancer RNA (eRNA)), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration that it does not adopt when the complex is not formed.
Integrity Index: The term “integrity index” as used herein refers to a value that is a quantitative representation of the frequency of a particular genomic complex, e.g., ASMC, across a relevant cell population (e.g., in a cell line or cell lines, or in cells of a given tissue type, e.g., from a particular subject). In some embodiments, across a relevant cell population comprises over a set time period (e.g., at a particular developmental/differentiation stage, at a certain disease/condition stage, or a certain time pre- or post-treatment with a therapeutic agent). The integrity index of a genomic complex, e.g., ASMC, for a cell population may be calculated by a variety of means, e.g., by either Formula 2 or 3 and the methods of Example 2. Integrity index may be abbreviated IntInd and may be expressed iteratively, e.g., the IntIndᵢ refers to the integrity index of genomic complex (e.g., ASMC) i.
Nucleating polypeptide: As used herein, the term “nucleating polypeptide” or “conjunction nucleating polypeptide” as used herein, refers to a protein that associates with an anchor sequence directly or indirectly and may interact with one or more conjunction nucleating polypeptides (that may interact with an anchor sequence or other nucleic acids) to form a dimer (or higher order structure) comprised of two or more such conjunction nucleating polypeptides, which may or may not be identical to one another. When conjunction nucleating polypeptides associated with different anchor sequences associate with each other so that the different anchor sequences are maintained in physical proximity with one another, the structure generated thereby is an anchor-sequence-mediated conjunction. That is, the close physical proximity of a nucleating polypeptide-anchor sequence interacting with another nucleating polypeptide-anchor sequence generates an anchor sequence-mediated conjunction (e.g., in some cases, a DNA loop), that begins and ends at the anchor sequence. As those skilled in the art, reading the present specification will immediately appreciate, terms such as “nucleating polypeptide”, “nucleating molecule”, “nucleating protein”, “conjunction nucleating protein”, may sometimes be used to refer to a conjunction nucleating polypeptide. As will similarly be immediately appreciated by those skilled in the art reading the present specification, an assembles collection of two or more conjunction nucleating polypeptides (which may, in some embodiments, include multiple copies of the same agent and/or in some embodiments one or more of each of a plurality of different agents) may be referred to as a “complex”, a “dimer” a “multimer”, etc.
Operably linked: As used herein, the phrase “operably linked” refers to a juxtaposition wherein the components described are in a relationship permitting them to function in their intended manner. A transcriptional control sequence “operably linked” to a functional element, e.g., gene, is associated in such a way that expression and/or activity of the functional element, e.g., gene, is achieved under conditions compatible with the transcriptional control sequence. In some embodiments, “operably linked” transcriptional control sequences are contiguous (e.g., covalently linked) with coding elements, e.g., genes, of interest; in some embodiments, operably linked transcriptional control sequences act in trans to or otherwise at a distance from the functional element, e.g., gene, of interest. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other.
Pharmaceutical composition: As used herein, the term “pharmaceutical composition” refers to an active agent (e.g., a modulating agent, e.g., a disrupting agent), formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces.
Proximal: As used herein, “proximal” refers to a closeness of two sites, e.g., nucleic acid sites, such that binding of an expression repressor at the first site and/or modification of the first site by an expression repressor will produce the same or substantially the same effect as binding and/or modification of the other site. For example, a DNA-targeting moiety may bind to a first site that is proximal to an enhancer (the second site), and the repressor domain associated with said DNA-targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, or proximal to an anchor sequence is less than 5000, 4000, 3000, 2000, 1000, 900, 800, 700, 600, 500, 400, 300, 200, 100, 50, or 25 base pairs from the target gene (e.g., an exon, intron, or splice site within the target gene), transcription control element, or anchor sequence (and optionally at least 20, 25, 50, 100, 200, or 300 base pairs from the target gene (e.g., an exon, intron, or splice site within the target gene), transcription control element, or anchor sequence).
Specific: As used herein, the term “specific” refers to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In some embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).
Specificity Index: The term “specificity index” as used herein refers to a value that is a quantitative representation of the rarity of a particular genomic complex, e.g., ASMC, across a plurality of cell populations (e.g., across a plurality of cell lines, or a plurality of tissue types, e.g., from a particular subject). In some embodiments, across a plurality of cell populations comprises over a set time period (e.g., at a particular developmental/differentiation stage, at a certain disease/condition stage, or a certain time pre- or post-treatment with a therapeutic agent). For example, the specificity index may be calculated for a given genomic complex (e.g., ASMC) in 10 exemplary cell populations (e.g., neuronal cells, muscle cells, liver cells, etc., e.g., of a subject). The lower the specificity index, the fewer cell populations the genomic complex (e.g., ASMC) detectably occurs in. The specifity index of a genomic complex, e.g., ASMC, may be calculated by a variety of means, e.g., by Formula 1 and the methods of Example 1. Specificity index may be abbreviated SpecInd and may be expressed iteratively, e.g., the SpecInd, refers to the specificity index of genomic complex (e.g., ASMC) i.
Stable/stability: As used herein, “stable” or “stability” refers to tendency of a particular interaction or set of interactions to be present over a period of time. As will be understood by those in the art, greater stability indicates greater tendency to be present over the relevant period of time and/or tendency to remain present over a longer period of time than a less stable interaction or set of interactions. In some embodiments, stability may be altered by altering one or more kinetic features of an interaction or set of interactions (e.g., on rate, off rate, etc); alternatively or additionally, in some embodiments, stability may be altered by altering one or more thermodynamic features of an interaction (e.g., energy level of an “interacting” state as compared with that of a “separated” state, and/or of a transition state between such interacting and separated states.
Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.
Target: An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence. In some embodiments, target binding is direct binding; in some embodiments, target binding may be indirect binding. In some embodiments, a modulating agent (e.g., disrupting agent) targets a genomic complex, e.g., ASMC, by binding to a component (e.g., polypeptide, nucleic acid, and/or genomic sequence element) of the genomic complex, e.g., ASMC.
Target gene: As used herein, the term “target gene” means a gene that is targeted for modulation, e.g., modulation of expression of the gene or modulation of epigenetic markers associated with the gene. In some embodiments, a target gene is part of a targeted genomic complex (e.g., a gene that has at least part of its genomic sequence as part of a target genomic complex, e.g., inside an anchor sequence-mediated conjunction), which genomic complex is targeted by one or more modulating (e.g., disrupting) agents as described herein. In some embodiments, a target gene is modulated by a genomic sequence of a target gene being directly contacted by a modulating (e.g., disrupting) agent as described herein. In some embodiments, a target gene is modulated by one or more components of a genomic complex of which it is part being contacted by a modulating (e.g., disrupting) agent as describe herein. In some embodiments, a target gene is outside of a target genomic complex, for example, is a gene that encodes a component of a target genomic complex (e.g., a subunit of a transcription factor). In some embodiments, a target gene is associated with a genomic complex as described herein.
Targeting moiety: As used herein, the term “targeting moiety” means an agent or entity that specifically targets, e.g., binds, a component or set of components that participate in a genomic complex as described herein (e.g., in an anchor sequence-mediated conjunction). In some embodiments, a targeting moiety in accordance with the present disclosure targets one or more component(s) of a genomic complex as described herein. In some embodiments, a targeting moiety targets a genomic sequence element (e.g., an anchor sequence). In some embodiments, a targeting moiety targets a genomic complex component other than a genomic sequence element. In some embodiments, a targeting moiety targets a plurality or combination of genomic complex components, which plurality may include a genomic sequence element. In some aspects, effective modulation, e.g., disruption, of a genomic complex (e.g., ASMC), as described herein, can be achieved by targeting a complex component other than a genomic sequence element. In some embodiments, a modulating (e.g., disrupting) agent as described herein modulates (e.g., disrupts) a target genomic complex (e.g., ASMC) by targeting at least one component of the target genomic complex.
Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) and/or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.
Transcriptional control sequence: As used herein, the term “transcriptional control sequence” as used herein, refers to a nucleic acid sequence that increases or decreases transcription of a gene, and includes (but is not limited to) a promoter and an enhancer. An “enhancing sequence” refers to a subtype of transcriptional control sequence and increases the likelihood of gene transcription. A “silencing or repressor sequence” refers to a subtypte of transcriptional control sequence and decreases the likelihood of gene transcription.

### DETAILED DESCRIPTION OF CERTAIN EMBODIMENTS

Provided herein are methods and compositions to modulate (e.g., disrupt) a genomic complex (e.g., ASMC) characterized by certain properties (e.g., having a particular integrity index and/or specificity index) using a modulating agent, e.g., disrupting agent. In some embodiments, modulating (e.g., disrupting) a genomic complex (e.g., ASMC) alters gene expression (e.g., within a cell, tissue, organism, etc.) of a gene associated with the targeted genomic complex (e.g., ASMC). Without wishing to be bound by theory, disruption of a genomic complex (e.g., ASMC) based on integrity index and/or specifity index allows for a more effective and tailored therapeutic approach. For example, selecting and disrupting a genomic complex (e.g., ASMC) having an integrity index greater than about 0.25 reduces the probability of altering expression of genes that may have undesirable target characteristics for disruption, such as genes which may be part of a genomic complex (e.g., ASMC) whose incidence is so low that such targeting is unlikely to achieve significant impact on expression of the gene. For example, selecting and disrupting a genomic complex (e.g., ASMC) having an integrity index greater than about 0.5 (e.g., 0.5-1) reduces the probability of altering expression of genes that may have undesirable target characteristics for disruption, such as genes which may be part of a genomic complex (e.g., ASMC) whose incidence is so low that such targeting is unlikely to achieve significant impact on expression of the gene. As another example, selecting and disrupting a genomic complex (e.g., ASMC) having an integrity index greater than or equal to about 0.25 and less than or equal to 0.75 reduces the probability of altering expression of genes that may have undesirable target characteristics for disruption, such as genes which may be part of a genomic complex (e.g., ASMC) whose incidence is so low that such targeting is unlikely to achieve significant impact on expression of the gene, or such as genes that may be part of a genomic complex (e.g., ASMC) whose incidence is so high (e.g., and interactions holding together said complex so strong) that modulation (e.g., disruption) of the complex is difficult or unlikely. As another example, compositions and methods as provided herein can be used to select and/or disrupt a genomic complex having a low integrity index in order to maintain or further lower their low integrity index.

A genomic complex (e.g., ASMC) may be targeted based on its integrity index and/or specificity index. In some embodiments, a targeted genomic complex (e.g., ASMC), as described herein, will be understood to refer to a complex at a particular (e.g., at a single particular) genomic site (e.g., gene or other genomic sequence element) having a particular integrity index (e.g., in a cell, tissue, organ, and/or subject). In some embodiments, a subset of genomic complexes (e.g., ASMCs) is characterized by a particular integrity index or range of indices. In some such embodiments, subset(s) of genomic complexes (e.g., ASMCs) may be targeted based on their observed incidence at a developmentally-specific period of time and/or in a cell-specific location. In some embodiments, a genomic complex (e.g., ASMC) having an integrity index not equivalent to the particular integrity index or outside the range of indices is a non-targeted genomic complex (e.g., ASMC) that may also exist in the same developmentally specific time periods and/or cell specific locations as a targeted genomic complex (e.g., ASMC), however, non-targeted genomic complexes (e.g., ASMCs) are not modulated (e.g., disrupted). In some embodiments, genomic complexes (e.g., ASMCs) characterized by a particular integrity index or range of indices are present in the same cell, tissue, organ, and/or subject as genomic complexes (e.g., ASMCs) not characterized by the particular integrity index or the range of indices. In some embodiments, genomic complexes (e.g., ASMCs) characterized by a particular integrity index or range of indices exist in a separate cell population from genomic complexes (e.g., ASMCs) not characterized by the particular integrity indices. The present disclosure provides, in part, technologies that achieve specific modulation of one or more genes in light of their operational proximity and/or relationship with a genomic complex (e.g., ASMC) characterized by a particular integrity index or range of indices.

In some embodiments, a genomic complex (e.g., ASMC) may be targeted based on its specificity index. In some embodiments, a target genomic complex (e.g., ASMC) is present in a target cell, tissue, or organ of a subject and is less prevalent (e.g., not present) in at least one non-target cell, tissue, or organ of a subject. In some embodiments, a targeted genomic complex (e.g., ASMC), as described herein, will be understood to refer to a complex at a particular (e.g., at a single particular) genomic site (e.g., gene or other genomic sequence element) having a particular specificity index (e.g., in a target cell, tissue, and/or organ, of a subject relative to one or more non-target or reference cells, tissues, and/or organs in the subject).

In some embodiments, a modulating (e.g., disrupting) agent is or comprises a targeting moiety that specifically targets a genomic complex (e.g., ASMC). In some embodiments, a genomic complex (e.g., ASMC) characterized by a particular integrity index or specificity index or range of indices is modulated (e.g., disrupted) by a modulating (e.g., disrupting) agent. In some embodiments, a genomic complex (e.g., ASMC) characterized by a particular integrity index, specificity index, or range of indices is not modulated (e.g., disrupted), however gene expression of a target gene associated with the targeted genomic complex (e.g., ASMC) is altered concomitant with or following an interaction between a modulating (e.g., disrupting) agent and the genomic complex (e.g., ASMC). In some embodiments, a modulating (e.g., disrupting) agent targets a genomic complex (e.g., ASMC) characterized by a particular integrity index and/or specificity index, wherein the modulating agent (e.g., disrupting agent) only has an effect, e.g., disruptive effect, on the targeted genomic complex (e.g., ASMC) and does not modulate (e.g., disrupt) genomic complexes not characterized by the particular integrity index, specificity index, or range of indices. In some embodiments, a modulating (e.g., disrupting) agent targets a genomic complex (e.g., ASMC) characterized by its presence in a target cell, tissue, or organ of a subject and its lower prevalence (e.g., lack of presence) in at least one non-target cell, tissue, or organ of a subject.

### Genomic Complexes

Genomic complexes relevant to the present disclosure include stable structures that comprise a plurality of polypeptide and/or nucleic acid (e.g., ribonucleic acid) components and that co-localize two or more genomic sequence elements (e.g., anchor sequences). In some embodiments, a genomic complex is or comprises an anchor sequence-mediated conjunction (ASMC). In some embodiments, genomic sequence elements that are co-localized in genomic complexes (e.g., ASMCs) relevant to the present disclosure include transcriptional control sequences, e.g., promoter, enhancer, and/or repressor sequences. Alternatively or additionally, in some embodiments, genomic sequence elements that are co-localized in genomic complexes (e.g., ASMCs) include binding sites for proteins that may act as nucleating polypeptides upon binding to the binding sites, such as, e.g., one or more of CTCF, YY1, etc. A genomic complex (e.g., ASMC) may comprise one or more polypeptide components and/or one or more non-genomic nucleic acid components.

In some embodiments, a genomic complex is characterized by its frequency of incidence using a quantitative measure such as an integrity index (e.g., as measured by Formula 2 or 3). In some embodiments, integrity index of a target genomic complex is calculated relative to the frequency of incidence of non-target genomic complexes and/or the frequency of incidence of all genomic complexes (e.g., ASMCs). In some embodiments, target genomic complexes have integrity index scores that allow them to be identified and/or targeted, relative to non-target genomic complexes.

Genomic sequence elements involved in genomic complexes as described herein, may be non-contiguous with one another. In some embodiments with noncontiguous genomic sequence elements (e.g., anchor sequences, promoters, and/or transcriptional regulatory sequences), a first genomic sequence element (e.g., anchor sequence, promoter, or transcriptional regulatory sequence) may be separated from a second genomic sequence element (e.g., anchor sequence, promoter, or transcriptional regulatory sequence) by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a first genomic sequence element (e.g., anchor sequence, promoter, or transcriptional/regulatory sequence) is separated from a second genomic sequence element (e.g., anchor sequence, promoter, or transcriptional regulatory sequence) by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween. In some embodiments, a genomic complex (e.g., ASMC) comprises a first genomic sequence element situated on a first chromosome and a second genomic sequence element situated on a second different chromosome.

### Genomic Sequence Elements

A genomic complex (e.g., ASMC) as described herein, when present, co-localizes two or more genomic sequence elements. In some embodiments, a genomic sequence element in a genomic complex (e.g., ASMC) is specifically bound by another component of the genomic complex (e.g., ASMC), for example a polypeptide component or non-genomic nucleic acid component. In some embodiments, a genomic sequence element may be or comprise an anchor sequence, a transcriptional control sequence (e.g., a promoter, an enhancer, or a silencing or repressor sequence), or a combination thereof. In some embodiments, a target genomic complex (e.g., ASMC) may be modulated (e.g., disrupted) by a modulating (e.g., disrupting) agent binding to or interacting with one or more genomic sequence elements. In some embodiments, a target genomic complex (e.g., ASMC) may be modulated (e.g., disrupted) by a modulating (e.g., disrupting) agent binding to or interacting with one or components that is not a genomic sequence element(s), e.g., a polypeptide component or a non-genomic nucleic acid component.

In some embodiments, a genomic sequence element that is included in a genomic complex (e.g., ASMC), does not comprise one or more of (e.g., all of) MYC, FOXJ3, TUSC5, DAND5, TTC21B, SHMT2, or CDK6, or a portion of any of the foregoing (e.g., a protein coding portion thereof, or a transcriptional control sequence associated with the foregoing). In some embodiments, a genomic complex, e.g., ASMC, does not comprise one or more of (e.g., all of) MYC, FOXJ3, TUSC5, DAND5, TTC21B, SHMT2, or CDK6, or a portion of any of the foregoing (e.g., a protein coding portion thereof, or a transcriptional control sequence associated with the foregoing).

Anchor Sequences

In general, an anchor sequence is a genomic sequence element to which a genomic complex component binds specifically. In some embodiments, binding to an anchor sequence nucleates genomic complex (e.g., ASMC) formation.

An anchor sequence-mediated conjunction (ASMC) comprises a plurality of anchor sequences, e.g., two or more anchor sequences. In some embodiments, anchor sequences can be manipulated or altered to modulate (e.g., disrupt) a naturally occurring genomic complex (e.g., ASMC) or to form a new genomic complex (e.g., ASMC) (e.g., to form a non-naturally occurring genomic complex (e.g., ASMC) with an exogenous or altered anchor sequence). Such alterations may modulate gene expression by, e.g., changing topological structure of DNA, e.g., thereby modulating (e.g., disrupting) the ability of a target gene to interact with gene regulation and control factors (e.g., a transcriptional control sequence, e.g., promoter, enhancer, or repressor sequence).

In some embodiments, chromatin structure is modified by substituting, adding or deleting one or more nucleotides within an anchor sequence. In some embodiments, chromatin structure is modified by substituting, adding, or deleting one or more nucleotides within an anchor sequence of an anchor sequence-mediated conjunction.

In some embodiments, an anchor sequence comprises a nucleating polypeptide binding motif, e.g., a CTCF-binding motif: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO:1), where N is any nucleotide.

A CTCF-binding motif may also be in an opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO:2).

In some embodiments, an anchor sequence comprises SEQ ID NO:1 or SEQ ID NO:2 or a sequence at least 75%, at least 80%, at least 90%, at least 95%, at least 96%, at least 97%, at least 98%, at least 99% identical to either SEQ ID NO:1 or SEQ ID NO:2.

In some embodiments, an anchor sequence-mediated conjunction comprises at least a first anchor sequence and a second anchor sequence. For example, in some embodiments, a first anchor sequence and a second anchor sequence may each comprise a nucleating polypeptide binding motif, e.g., each comprises a CTCF binding motif.

In some embodiments, a first anchor sequence and second anchor sequence comprise different sequences, e.g., a first anchor sequence comprises a CTCF binding motif and a second anchor sequence comprises an anchor sequence other than a CTCF binding motif. In some embodiments, each anchor sequence comprises a nucleating polypeptide binding motif and one or more flanking nucleotides on one or both sides of a nucleating polypeptide binding motif.

Two CTCF-binding motifs (e.g., contiguous or non-contiguous CTCF binding motifs) that can form an ASMC may be present in a genome in any orientation, e.g., in the same orientation (tandem) either 5′-3′ (left tandem, e.g., the two CTCF-binding motifs that comprise SEQ ID NO:1) or 3′-5′ (right tandem, e.g., the two CTCF-binding motifs comprise SEQ ID NO:2), or convergent orientation, where one CTCF-binding motif comprises SEQ ID NO:1 and another other comprises SEQ ID NO:2. CTCFBSDB 2.0: Database For CTCF binding motifs And Genome Organization (http://insulatordb.uthsc.edu/) can be used to identify CTCF binding motifs associated with a target gene.

In some embodiments, an anchor sequence comprises a CTCF binding motif associated with a target gene, wherein the target gene is associated with a disease, disorder and/or condition.

In some embodiments, methods of the present disclosure comprise modulating, e.g., disrupting, a genomic complex (e.g., ASMC), e.g., by modifying chromatin structure, by substituting, adding, or deleting one or more nucleotides within an anchor sequence, e.g., a nucleating polypeptide binding motif. One or more nucleotides may be specifically targeted, e.g., a targeted alteration, for substitution, addition or deletion within an anchor sequence, e.g., a nucleating polypeptide binding motif.

In some embodiments, a genomic complex (e.g., ASMC) may be altered by changing an orientation of at least one nucleating polypeptide binding motif. In some embodiments, an anchor sequence comprises a nucleating polypeptide binding motif, e.g., CTCF binding motif, and a targeting moiety introduces an alteration in at least one nucleating polypeptide binding motif, e.g., altering binding affinity for a nucleating polypeptide.

Transcriptional Control Sequences

In some embodiments, a genomic complex (e.g., ASMC) colocalizes two or more genomic sequence elements that include one or more transcriptional control sequences. Those skilled in the art are familiar with a variety of positive (e.g., promoters or enhancers) or negative (e.g., repressors or silencers) transcriptional control sequences that are associated with genes. Typically, when a cognate regulatory protein is bound to such a transcriptional regulatory sequence, transcription from the associated gene(s) is altered (e.g., increased for a positive regulatory sequence; decreased for a negative regulatory sequence).

Promoter Sequences

In some embodiments, a genomic complex (e.g., ASMC) colocalizes two or more genomic sequence elements, wherein the two or more genomic sequence elements include a promoter. Those skilled in the art are aware that a promoter is, typically, a sequence element that initiates transcription of an associated gene. Promoters are typically near the 5′ end of a gene, not far from its transcription start site.

As those of ordinary skill are aware, transcription of protein-coding genes in eukaryotic cells is typically initiated by binding of general transcription factors (e.g., TFIID, TFIIE, TFIIH, etc) and Mediator to core promoter sequences as a preinitiation complex that directs RNA polymerase II to the transcription start site, and in many instances remains bound to the core promoter sequences even after RNA polymerase escapes and elongation of the primary transcript is initiated.

In many embodiments, a promoter includes a sequence element, such as TATA, Inr, DPE, or BRE, but those skilled in the art are well aware that such sequences are not necessarily required to define a promoter.

### Polypeptide Components

In some embodiments, a genomic complex (e.g., ASMC) comprises one or more polypeptide components. A polypeptide component, e.g., transcription machinery and/or regulatory factors, may be targeted as a way to modulate a genomic complex (e.g., ASMC) containing the polypeptide component. In some embodiments, targeting a polypeptide component alters the structure and/or function of the polypeptide component. In some embodiments, targeting a polypeptide component alters the extent of genomic complex (e.g., ASMC) formation, e.g., the level of genomic complex (e.g., ASMC) present comprising the polypeptide component. In some embodiments, polypeptide components are targeted to alter the association of a non-genomic nucleic acid component with a genomic sequence element of a target genomic complex (e.g., ASMC). In some embodiments, targeting a polypeptide component as described herein changes the frequency and/or duration of association between the polypeptide component and a genomic sequence element of a target genomic complex (e.g., ASMC). In some embodiments, changes to the frequency and/or duration of association between a polypeptide component and a genomic sequence element may modulate (e.g., disrupt) a target genomic complex (e.g., ASMC). In some embodiments, modulating (e.g., disrupting) a target genomic complex (e.g., ASMC) comprises changing (e.g., decreasing) the frequency and/or duration of association between a polypeptide component and a genomic sequence element.

Nucleating Polypeptides

In some embodiments, a genomic complex (e.g., ASMC) comprises a polypeptide component that is or comprises a nucleating polypeptide. A nucleating polypeptide may promote formation of an anchor sequence-mediated conjunction. Nucleating polypeptides that may be targeted by modulating (e.g., disrupting) agents as described herein may include, for example, proteins (e.g., CTCF, USF1, YY1, TAF3, ZNF143, etc) that bind specifically to anchor sequences, or other proteins (e.g., transcription factors) whose binding to a particular genomic sequence element may initiate formation of a genomic complex (e.g., ASMC) as described herein. In some embodiments, a modulating (e.g., disrupting) agent may target one or more anchor sequences or genomic sequence elements to which nucleating polypeptides may bind in a target genomic complex (e.g., ASMC). In some embodiments, a modulating (e.g., disrupting) agent may target (e.g., bind) to a nucleating polypeptide.

A nucleating polypeptide may be, e.g., CTCF, cohesin, USF1, YY1, TATA-box binding protein associated factor 3 (TAF3), ZNF143 binding motif, or another polypeptide that promotes formation of an anchor sequence-mediated conjunction. A nucleating polypeptide may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. A nucleating polypeptide may modulate DNA interactions within or around the anchor sequence-mediated conjunction. For example, a nucleating polypeptide can recruit other factors to an anchor sequence, such that alteration (e.g. disruption) of an anchor sequence-mediated conjunction occurs.

A nucleating polypeptide may also have a dimerization domain for homo- or heterodimerization. One or more nucleating polypeptides, e.g., endogenous and engineered, may interact to form an anchor sequence-mediated conjunction. In some embodiments, a modulating agent, e.g., disrupting agent, disrupts a target genomic complex (e.g., ASMC) by interfering with (e.g. directly or indirectly) this interaction. In some embodiments, a nucleating polypeptide is engineered to further include a stabilization domain, e.g., cohesion interaction domain, to stabilize an anchor sequence-mediated conjunction. In some embodiments, a nucleating polypeptide is engineered to bind a target sequence, e.g., target sequence binding affinity is modulated. In some embodiments, a nucleating polypeptide is selected or engineered with a selected binding affinity for an anchor sequence within an anchor sequence-mediated conjunction.

Nucleating polypeptides and their corresponding anchor sequences may be identified through use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChIA-PET analysis using a bait, such as Cohesin, YY1 or USF1, ZNF143 binding motif, and MS to identify complexes that are associated with a bait.

In some embodiments, a nucleating polypeptide has a binding affinity for an anchor sequence greater than or less than a reference value, e.g., binding affinity for an anchor sequence in absence of an alteration. In some embodiments, a nucleating polypeptide is modulated to alter (e.g. disrupt) its interaction with an anchor sequence-mediated conjunction, e.g. its binding affinity for an anchor sequence within an anchor sequence-mediated conjunction.

Transcription Machinery

In some embodiments, a genomic complex (e.g., ASMC) comprises one or more components of the transcription machinery of a cell. Those skilled in the art are familiar with proteins that participate as part of the transcription machinery involved in transcribing a particular gene (e.g., a protein-coding gene). For example, RNA polymerase (e.g., RNA polymerase II), general transcription factors such as TFIIA, TFIIB, TFIID, TFIIE, TFIIF, and TFIIH, Mediator, certain elongation factors, etc.

In some embodiments, methods described herein (e.g., of modulating, e.g., disrupting a genomic complex (e.g., ASMC) comprise targeting a component of the transcription machinery. Targeting one or more components of transcription machinery involved in a particular genomic complex (e.g., ASMC) may alter expression of one or more genes associated with the genomic complex (e.g., ASMC). For example, in some embodiments, targeting a transcription machinery component of a target genomic complex (e.g., ASMC) may modulate (e.g., disrupt) the genomic complex, e.g., by modulating (e.g., disrupting) or otherwise interfering with interactions between the targeted component and one or more other components of the genomic complex.

Transcription Regulators

In some embodiments, technologies provided herein modulate (e.g., disrupt) a particular genomic complex (e.g., ASMC) by targeting a transcription regulatory protein involved or otherwise associated with the genomic complex (e.g., ASMC). In some embodiments, a modulating (e.g., disrupting) agent modulates a particular genomic complex (e.g., ASMC) by interacting with a transcription regulatory protein such that the genomic complex (e.g., ASMC) no longer comprises the transcription regulatory protein, e.g., by preventing the transcription regulatory protein from interacting with one or more other components of the genomic complex (e.g., ASMC).

Those skilled in the art are aware of a large variety of transcriptional regulatory proteins, many of which are DNA binding proteins (e.g., containing a DNA binding domain such as a helix-loop-helix motif, ETS, a forkhead, a leucine zipper, a Pit-Oct-Unc domain, and/or a zinc finger), many of which interact with core transcriptional machinery by way of interaction with Mediator. In some embodiments, a transcriptional regulatory protein may be or comprise an activator (e.g., that may bind to an enhancer). In some embodiments, a transcriptional regulatory protein may be or comprise a repressor (e.g., that may bind to a silencer).

In some embodiments, targeting a transcription regulatory protein may modulate (e.g., disrupt) a genomic complex (e.g., ASMC), for example by interfering with interactions between the targeted transcription regulatory protein and one or more other components (e.g., with Mediator, or a genomic sequence element to which the transcription regulatory protein binds).

### Non-Genomic Nucleic Acid Components

In some embodiments, a genomic complex (e.g., ASMC) comprises a non-genomic nucleic acid component. In some embodiments, the present disclosure provides technologies for modulating (e.g., disrupting) a genomic complex (e.g., ASMC), e.g., altering the level of the genomic complex, by targeting a non-genomic nucleic acid component of the complex. In some embodiments, the non-genomic nucleic acid component is or comprises an RNA.

For example, those skilled in the art will be aware that genomic complexes may include one or more non-coding RNAs (ncRNAs) such as one or more enhancer RNAs (eRNAs). Those skilled in the art will be aware that eRNAs are typically transcribed from enhancers, and may participate in regulating expression of one or more genes regulated by the enhancer (i.e., target genes of the enhancer). In some embodiments, a genomic complex (e.g., ASMC) comprises an eRNA, an enhancer (e.g., from which the eRNA was transcribed), a promoter (e.g., operably linked to a target gene, e.g., a gene whose expression will be modulated by modulation of the genomic complex). In some embodiments, a genomic complex (e.g., ASMC) comprises an eRNA, an enhancer, a promoter (e.g., operably linked to a target gene), and the eRNA is involved in the genomic complex via, for example, interactions with one or more anchor sequence nucleating polypeptides such as CTCF and YY1, general transcription machinery components, Mediator, and/or one or more sequence-specific transcriptional regulatory agents such as p53 or Oct4. In some embodiments, modulation (e.g., disruption) of a genomic complex (e.g., ASMC) may occur, by targeting a non-coding RNA, e.g., eRNA.

Without being bound by theory, it is contemplated that modulation (e.g., disruption) of a genomic complex (e.g., ASMC) may alter the level of an eRNA, which may, in some embodiments, alter (e.g., decrease) the level of expression of a target gene. In some embodiments, a modulating agent (e.g., disrupting agent) may comprise a component that targets one or more eRNAs. In some embodiments, knockdown of an eRNA may cause knockdown of a target gene.

### Anchor Sequence-Mediated Conjunction (ASMC)

In some embodiments, a genomic complex is or comprises an anchor sequence-mediated conjunction (ASMC). In some embodiments, an anchor sequence-mediated conjunction is formed when nucleating polypeptide(s) bind to anchor sequences in the genome and interactions between and among these proteins and, optionally, one or more other components (e.g., polypeptide components and/or non-genomic nucleic acid components), forms a conjunction in which the anchor sequences are physically co-localized. In some embodiments, one or more genes is associated with an anchor sequence-mediated conjunction. In some embodiments, the anchor sequence-mediated conjunction includes one or more anchor sequences, one or more genes, and one or more transcriptional control sequences, such as an enhancing or silencing sequence. In some embodiments, a transcriptional control sequence is within, partially within, or outside an anchor sequence-mediated conjunction.

In some embodiments, a genomic complex (e.g., an anchor sequence-mediated conjunction) comprises a first anchor sequence, a nucleic acid sequence (e.g., a gene), a transcriptional control sequence, and a second anchor sequence. In some embodiments, a genomic complex (e.g., ASMC) comprises, in order: a first anchor sequence, a transcriptional control sequence, and a second anchor sequence; or a first anchor sequence, a nucleic acid sequence (e.g., a gene), and a second anchor sequence. In some embodiments, either one or both of the nucleic acid sequence (e.g., gene) and the transcriptional control sequence is located within or outside the genomic complex (e.g., ASMC). In some embodiments, a genomic complex (e.g., an anchor sequence-mediated conjunction) includes a TATA box, a CAAT box, a GC box, or a CAP site.

In some embodiments, a genomic complex (e.g., ASMC) colocalizes two genomic sequence elements that are within, partially within, or contiguous with (i) a gene whose expression is modulated (e.g., decreased or increased) by the formation or disruption of the genomic complex; and/or (ii) one or more transcriptional control sequences operably linked to the gene.

The present disclosure is directed, in part, to methods of modulating (e.g., disrupting) a genomic complex, e.g., ASMC, using a modulating agent (e.g., disrupting agent) described herein. In some embodiments, a modulating (e.g., disrupting) agent may modulate transcription of a target gene associated with an ASMC. For example, in some embodiments, transcription of a target gene is activated by its inclusion in an activating ASMC or exclusion from a repressive ASMC; in some embodiments a modulating (e.g., disrupting) agent causes a target gene to be included in an activating ASMC or excluded from a repressive ASMC. In some embodiments, a modulating (e.g., disrupting) agent may cause an anchor sequence-mediated conjunction to comprise a transcriptional control sequence that increases transcription of a nucleic acid sequence (e.g., gene), where the ASMC did not comprise the transcriptional control sequence prior to modulation. In some embodiments, a modulating (e.g., disrupting) agent may cause an anchor sequence-mediated conjunction to exclude a transcriptional control sequence that decreases transcription of a nucleic acid sequence (e.g., gene), where the ASMC comprised the transcriptional control sequence prior to modulation.

In some embodiments, transcription of a target gene is repressed by its inclusion in a repressive ASMC or exclusion from an activating ASMC. In some such embodiments, a modulating (e.g., disrupting) agent causes a target gene to be excluded from an activating ASMC or included in a repressive ASMC. In some embodiments, an anchor sequence-mediated conjunction includes a transcriptional control sequence that decreases transcription of a nucleic acid sequence (e.g., gene). In some embodiments, an anchor sequence-mediated conjunction excludes a transcriptional control sequence that increases transcription of a nucleic acid sequence (e.g., gene).

An “activating ASMC” is an ASMC that is open to active gene transcription, for example, an ASMC comprising a transcriptional control sequence (e.g., a promoter or enhancer) that enhances transcription of an operably linked nucleic acid sequence (e.g., gene). A “repressive ASMC”, is an ASMC that is closed off from active gene transcription, for example, an ASMC comprising a transcriptional control sequence (e.g., a repressor sequence) that represses transcription of an operably linked nucleic acid sequence (e.g., gene). In some embodiments, an ASMC (e.g., an activating ASMC) comprises a gene and an operably linked enhancer and the gene is actively expressed. In some embodiments, an ASMC (e.g., an activating ASMC) comprises a gene and a repressor sequence is situated outside the ASMC, wherein the gene is actively expressed. In some embodiments, an ASMC (e.g., a repressive ASMC) comprises a gene and an operably linked repressor sequence situated within the ASMC and the gene is not actively expressed. In some embodiments, an ASMC (e.g., a repressive ASMC) comprises a gene and an enhancer is situated outside the ASMC, wherein the gene is not actively expressed. In some embodiments, an ASMC (e.g., an activating ASMC) comprises a gene and an operably linked enhancer, wherein a repressor is situated outside the ASMC and the gene is actively expressed. In some embodiments, an ASMC (e.g., a repressive ASMC) comprises a gene and an operably linked repressor sequence, wherein an enhancer situated outside the ASMC and the gene is not actively expressed.

Types of ASMCs

In some embodiments, a genomic complex (e.g., ASMC) comprises or partially comprises one or more, e.g., 2, 3, 4, 5, or more, genes.

In some embodiments, an anchor sequence-mediated conjunction comprises or partially comprises one or more, e.g., 2, 3, 4, 5, or more, transcriptional control sequences. In some embodiments, a target gene is non-contiguous with one or more transcriptional control sequences. In some embodiments where a gene is non-contiguous with its transcriptional control sequence(s), a gene may be separated from one or more transcriptional control sequences by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a gene is separated from a transcriptional control sequence by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

Without wishing to be bound by theory, it is contemplated that in some embodiments, understanding (e.g., identifying or classifying) whether an ASMC is or corresponds to a particular type of anchor sequence-mediated conjunction may help to determine how to modulate gene expression by altering the ASMC, e.g., influencing the choice of DNA-binding moiety or effector moiety. For example, in some embodiments, some types of anchor sequence-mediated conjunctions comprise one or more transcriptional control sequences (e.g., an enhancer) within an anchor sequence-mediated conjunction. Modulation (e.g., disruption) of such an ASMC by modulating (e.g., disrupting) the genomic complex comprising the ASMC and/or modulating (e.g., disrupting) presence of the ASMC within a genomic complex, e.g., altering one or more anchor sequences wherein such an alteration results in a disrupted ASMC, is likely to decrease transcription of a target gene within the genomic complex and/or ASMC. In some embodiments, modulation (e.g., disruption) of a repressive ASMC, or a genomic complex comprising the ASMC, results in increased gene expression. In some embodiments, modulation (e.g., disruption) of an activating ASMC, or a genomic complex comprising the ASMC, results in decreased gene expression.

By way of non-limiting example, ASMCs may be categorized by certain structural features and types. As further described herein, in some embodiments, certain types of ASMCs may be modulated (e.g., disrupted) in particular ways, in order to effect certain structural features (e.g., DNA topology). In some embodiments, changes in structural features may alter post-nucleating activities and programs associated with the genomic complex (e.g., ASMC). In some embodiments, changes in structural features may result from changes to proteins or non-coding sequences that are part of a genomic complex (e.g., ASMC) but not part of a gene itself. In some embodiments, changes in non-structural (e.g., functional) features associated with the genomic complex (e.g., ASMC) in the absence of structural changes may result from changes to proteins or non-coding sequences.

Type 1

In some embodiments, an anchor sequence-mediated conjunction comprises one or more genes and one or more transcriptional control sequences. For example, a target gene and one or more transcriptional control sequences may be located within, at least partially, an anchor sequence-mediated conjunction. Such an ASMC may be referred to herein as a Type 1 ASMC. In some embodiments, disruption of a Type 1 ASMC disrupts accessibility, e.g., operable linkage, of the one or more genes and one or more transcriptional control elements comprised or partially comprised within the Type 1 ASMC.

In some embodiments, a target gene has a defined state of expression, e.g., in its native state, e.g., in a diseased state. For example, a target gene may have a high level of expression and be part of an ASMC, e.g., Type 1 ASMC, comprising or partially comprising the target gene and one or more transcriptional control sequences. By modulating (e.g., disrupting) the ASMC (e.g., Type 1 ASMC), expression of the target gene may be decreased, e.g., transcription of the target gene may be decreased due to conformational changes of DNA previously open to transcription within the ASMC, e.g., decreased transcription due to conformational changes of DNA creating additional distance between the target gene and the one or more transcriptional control sequences (e.g., an enhancer). In some embodiments, disruption of a Type 1 ASMC decreases or abolishes the operable linkage between a transcriptional control sequence (e.g., an enhancer) and a target gene, e.g., thereby decreasing expression of the target gene.

In some embodiments, an ASMC, e.g., Type 1 ASMC, comprises a target gene and one or more transcriptional control sequences (e.g., an enhancer). In some embodiments, modulation (e.g., disruption) of the ASMC decreases expression of the target gene.

In some embodiments, an ASMC, e.g., Type 1 ASMC, comprises a target geneand one or more transcriptional control sequences (e.g., an enhancer) are accessible to, e.g., are operably linked to, the target gene, wherein the transcriptional control sequence(s) reside at least partially (and optionally, less than entirely) within the ASMC. In some embodiments, an ASMC, e.g., Type 1 ASMC, comprises one or more transcriptional control sequences (e.g., an enhancer) and one or more target genes are accessible to, e.g., are operably linked to, the transcriptional control sequence(s), wherein the one or more target genes reside at least partially (and optionally, less than entirely) within the ASMC. In some embodiments, modulation (e.g., disruption) of the ASMC decreases expression of the target gene.

In some embodiments, modulation (e.g., disruption) of an anchor sequence-mediated conjunction, e.g., a Type 1 ASMC, decreases expression of a gene. For example, an exemplary Type 1 anchor sequence-mediated conjunction comprises a gene encoding MYC and disruption of an exemplary Type 1 anchor sequence-mediated conjunction decreases expression of the MYC gene and MYC protein levels.

In some embodiments, an exemplary Type 1 anchor sequence-mediated conjunction comprises a gene encoding Foxj3 and modulation (e.g., disruption) of an exemplary Type 1 anchor sequence-mediated conjunction decreases expression of the Foxj3 gene and Foxj3 protein levels.

Type 2

In some embodiments, an ASMC comprises one or more genes and does not comprise one or more transcriptional control sequences which are situated such that the transcriptional control sequences are not accessible to (e.g., not operably linked to) the one or more genes in the presence of the ASMC. In some embodiments, an ASMC comprises one or more transcriptional control sequences and does not comprise one or more genes which are situated such that the transcriptional control sequences are not accessible to (e.g., not operably linked to) the one or more genes in the presence of the ASMC. For example, an anchor sequence-mediated conjunction may comprise a target gene and the ASMC modulates (e.g., prevents or inhibits) the ability of one or more transcriptional control sequences to regulate, modulate, or influence expression of the target gene. Transcriptional control sequences may be separated from a given gene, e.g., reside on the opposite side, at least partially, e.g., inside or outside, of an anchor sequence-mediated conjunction. Such an ASMC may be referred to herein as a Type 2 ASMC. In some embodiments, disruption of a Type 2 ASMC makes the one or more genes and one or more transcriptional control sequences accessible to (e.g., operably linked to) one another, such that a transcriptional control element may modulate expression of the gene.

In some embodiments, a gene is enclosed within an anchor sequence-mediated conjunction (e.g., Type 2 ASMC), while a transcriptional control sequence (e.g., enhancing sequence) is not enclosed within an anchor sequence-mediated conjunction (e.g., Type 2 ASMC). In some embodiments, a transcriptional control sequence (e.g., enhancing sequence) is enclosed within an anchor sequence-mediated conjunction (e.g., Type 2 ASMC), while a gene is not enclosed within an anchor sequence-mediated conjunction (e.g., Type 2 ASMC).

In some embodiments, a gene is inaccessible to one or more transcriptional control sequences due to an anchor sequence-mediated conjunction, and modulation (e.g., disruption) of an anchor sequence-mediated conjunction (e.g., a Type 2 ASMC) allows one or more transcriptional control sequences to regulate, modulate, or influence expression of a gene.

In some embodiments, a gene is inside and outside (e.g., partially inside and partially outside) an anchor sequence-mediated conjunction (e.g., Type 2 ASMC) and inaccessible to one or more transcriptional control sequences. Modulation (e.g., disruption) of an anchor sequence-mediated conjunction (e.g., Type 2 ASMC) increases access of transcriptional control sequences to regulate, modulate, or influence expression of a gene, e.g., transcriptional control sequences increase expression of a gene.

In some embodiments, a gene is inside an anchor sequence-mediated conjunction (e.g., Type 2 ASMC) and inaccessible to one or more transcriptional control sequences residing outside, at least partially, an anchor sequence-mediated conjunction (e.g., Type 2 ASMC). Modulation (e.g., disruption) of a given anchor sequence-mediated conjunction (e.g., Type 2 ASMC) increases expression of a given gene.

In some embodiments, a gene is outside, at least partially, of an anchor sequence-mediated conjunction (e.g., Type 2 ASMC) and inaccessible to one or more transcriptional control sequences residing inside an anchor sequence-mediated conjunction (e.g., Type 2 ASMC). Modulation (e.g., disruption) of a given anchor sequence-mediated conjunction (e.g., Type 2 ASMC) increases expression of a given gene.

In some embodiments, a target gene has a defined state of expression, e.g., in its native state, e.g., in a diseased state. For example, a target gene may have a moderate to low level of expression. By modulating (e.g., disrupting) an anchor sequence-mediated conjunction (e.g., Type 2 ASMC), expression of a target gene may be modulated, e.g., increased transcription due to conformational changes of DNA previously closed to transcription within an anchor sequence-mediated conjunction (e.g., Type 2 ASMC), e.g., increased transcription due to conformational changes of DNA by bringing transcriptional control sequences (e.g., an enhancer) into closer association with (e.g., operable linkage) to a given target gene.

Detecting Genomic Complexes

The present disclosure is directed, in part, to methods comprising measuring or identifying the presence, quantity, stability, configuration, and/or localization of a genomic complex (e.g., ASMC) by one or more assays. In some embodiments, a given genomic complex (e.g., ASMC) is at a particular genomic site (e.g., bound to a particular genomic sequence element) in a certain measurable quantity or configuration and administration of a modulating agent, e.g., disrupting agent, may change (e.g., increase or decrease) the amount of genomic complex (e.g., ASMC) present at a particular genomic site.

### Assays

Assays known to those of skill in the art and/or described herein may be conducted to determine the presence, quantity, stability, configuration, and/or localization of a genomic complex (e.g., ASMC) (e.g., integrity index of a particular loop type). In some embodiments, assays are conducted to determine if modulation, e.g., disruption, of a genomic complex (e.g., ASMC) has been successful. In some embodiments, an assay may determine the localization of a genomic complex (e.g., ASMC). In some embodiments, an assay may provide data to determine the specificity and/or integrity index of a genomic complex (e.g., ASMC). In some embodiments, an assay provides structural information, e.g., is a structural readout, about the genomic complex (e.g., ASMC). In some embodiments, an assay provides functional information, e.g., is a functional readout, about the genomic complex (e.g., ASMC).

In some embodiments, an assay is or comprises quantifying the amount of a genomic complex (e.g., ASMC) in a given cell(s) or cell type and/or at a given developmental stage and/or at a given point in time and/or over a given period of time. Such assays may be selected from but are not limited to chromatin immunoprecipitation (ChIP), immunostaining, and fluorescent in situ hybridization (FISH). In some embodiments, assays (e.g., immunostaining) may visualize presence of a particular disrupting agent and/or genomic complex. In some embodiments, assays (e.g., fluorescent in situ hybridization assays (FISH)) may both visualize and localize presence of a particular disrupting agent and/or genomic complex. In some embodiments, an assay comprises a step of immunoprecipitation, e.g., chromatin immunoprecipitation, to detect the state (e.g., present vs not present) of a particular genomic complex (e.g., ASMC). In some embodiments, an assay comprises performing one or more serial chromatin immunoprecipitations, e.g., at least a first chromatin immunoprecipitation using an antibody against a first component of a targeted genomic complex (e.g., ASMC), a second chromatin immunoprecipitation using an antibody against a second component of a targeted genomic complex (e.g., ASMC), and optionally a step to determine presence and/or level of a genomic sequence element that is in proximity to the genomic complex (e.g., ASMC) (e.g., a PCR assay).

In some embodiments, an assay is or comprises a chromosome conformation capture assay. In some embodiments, a chromosome capture assay (e.g., a “one vs. one” assay, e.g., a 3C assay detects presence and/or level of interactions between a single pair of genomic loci). In some embodiments, a chromosome capture assay (e.g., a “one vs. many or all” assay, e.g., a 4C assay) detects presence and/or level of interactions between one genomic locus and multiple and/or all other genomic loci. In some embodiments, a chromosome capture assay (e.g., a “many vs. many” assay, e.g., a 5C assay) detects presence and/or level of interactions between multiple and/or many genomic loci within a given region. In some embodiments, a chromosome capture assay (e.g., an “all vs. all” assay, e.g., a Hi-C assay) detects presence and/or level of interactions between all or nearly all genomic loci.

In some embodiments, an assay comprises a step of cross-linking cell genomes (e.g., using formaldehyde). In some embodiments, an assay comprises a capture step (e.g., using an oligonucleotide) to enrich for specific loci or for a specific locus of interest. In some embodiments, an assay is a single-cell assay.

In some embodiments, an assay combines chromatin immunoprecipitation (ChIP) of CTCF with chromatin conformation capture methods (e.g., HiC) and with massively parallel DNA sequencing to identify instances of CTCF-dependent looping of genomic loci (“CTCF HiChIP” as described in doi-10.1038/nmeth.3999).

In some embodiments, an assay detects interactions between genomic loci at a genome-wide level, e.g., a Chromatin Interaction Analysis by Paired-End Tag Sequencing (ChiA-PET) assay.

Specifically, in some embodiments, assays may include, e.g., ChIA pet analysis in specific cell populations and/or in specific tissues and/or at particular developmental timepoints within a given cell population and/or tissue. For example, in some embodiments, ChIA pet analysis may be able to determine which percentage of a given cell population has a particular genomic complex (e.g., ASMC) in the “present” state at the time a particular experiment took place. For example, a particular experiment may take place after an integrity index does not/cannot/will not change due to, e.g. fixation of cells or, e.g. after an event that locks chromatin into an irreversible state.

In some embodiments, an assay may comprise ChIP with molecules known to be capable of functioning as anchor in anchor-sequence-mediated conjunctions (e.g., CTCF, cohesin, etc.). In some such embodiments, the ChIP assay may be able to determine occupancy of certain factors (e.g., genomic complex components) on particular portions of genomic DNA regardless of whether an anchor sequence-mediated conjunction is present. In some embodiments, such a determination can provide an estimate of potential loop formation.

In some embodiments, an assay may include a genome-wide analysis in a particular organism of interest to determine location and frequency of CTCF binding motifs. In some embodiments, such a determination can provide a “map” of potential sites of genomic complex (e.g., loop) formation.

In some embodiments, any assay as described herein may be performed in two or more different tissues or two or more different cell types (e.g., cells at different developmental stages) and results compared between different tissues or cell types (e.g., developmental stages). Without being bound by any particular theory, the present disclosure contemplates that certain genomic complexes may be present in a particular tissue and/or particular developmental stage, but absent in another tissue and/or developmental stage and such a comparison of presence or absence will provide information to calculate integrity index scores. In some embodiments, absence of a particular genomic complex will result in an integrity index score of zero.

### Integrity Index

The present disclosure is directed, in part, to methods of modulating, e.g., disrupting, a genomic complex (e.g., ASMC), wherein the genomic complex (e.g., ASMC) has or is identified as having an integrity index of a particular value or within a range of values. The integrity index is a value that is a quantitative representation of the frequency of a particular genomic complex (e.g., ASMC) across a relevant cell population. The integrity index may be calculated, e.g., by either Formula 2 or Formula 3 as described herein.

Without wishing to be bound by theory, the present disclosure contemplates that interactions between and/or among components of a genomic complex (e.g., ASMC) are dynamic and vary in strength, frequency of incidence, and stability, resulting in genomic complexes (e.g., ASMCs) that vary in their frequency of incidence and in their stability (e.g., the extent to which a genomic complex (e.g., ASMC) “breathes”, e.g., forms, dissociates, and forms again in repeated cycles) within a cell population (e.g., between cells of a cell population).

A genomic complex (e.g., ASMC) with a high integrity index occurs in, e.g., is more prevalent in, more cells of the cell population than a genomic complex (e.g., ASMC) with a low integrity index. A genomic complex (e.g., ASMC) with a high integrity index may “breathe” less than a genomic complex (e.g., ASMC) with a low integrity index, e.g., the high index genomic complex may more stably remain associated and not dissociate as frequently as a low index genomic complex. For example, a genomic complex (e.g., ASMC) with an integrity index of 0 does not appreciably occur (e.g., does not occur) in the cell population. For example, a genomic complex (e.g., ASMC) with an integrity index of 1 is present in essentially all (e.g., all) cells of the population. A genomic complex (e.g., ASMC) with an integrity index of 0.5 may be present in about half of cells in the population at a given time, e.g., may be permanently dissociated (not present) in half of cells and permanently associated (present) in the other half; present in all cells 50% of the time (e.g., the genomic complex (e.g., ASMC) “breathes”, e.g., cycles between formation and dissociation, frequently); or present/not present in cells over time in a distribution that produces index value of 0.5 (e.g., one of skill will understand that many distributions over time of cells having or not having the genomic complex (e.g., ASMC) of interest may produce a value of 0.5).

In some embodiments, the integrity index of a target genomic complex (e.g., ASMC) is the lower of: i) a ratio of the frequency of incidence of a target genomic complex (e.g., ASMC) in a cell population to a normalization factor; or ii) 1, where that normalization factor is a high percentile value (e.g., 90, 91, 92, 93, 94, 95, 96, 97, 98, or 99^{th} percentile) of the frequency of incidence of all genomic complexes (e.g., ASMCs) in the cell population (e.g., the integrity index as determined by Formula 2). In some embodiments, the normalization factor is the 95^{th} percentile frequency of incidence of all genomic complexes (e.g., ASMCs) in the cell population (e.g., as seen in Formula 2 and measured by the method of Example 2). Without wishing to be bound by theory, it may be advantageous to use a high percentile value of the frequency of all genomic complexes (e.g., ASMCs), as opposed to the highest genomic complex (e.g., ASMC) frequency observed in a cell population, to avoid the issue of very stable and/or omnipresent outlier genomic complexes (e.g., ASMCs). The frequency of incidence of a genomic complex (e.g., ASMC) in a cell population may be measured, e.g., by an experimental technique such as ChIA-PET, HiChIP, HiC, or 4C-seq. In some embodiments, the integrity index of a target genomic complex (e.g., ASMC) i is determined by Formula 2:

$${IntInd}_{i} = min ( \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} , 1 )$$

In some embodiments, the integrity index of a target genomic complex (e.g., ASMC) is the lower of: i) the ratio of the base 2 logarithm of the number of paired end tag (PET) reads supporting the presence of the genomic complex (e.g., ASMC) to a normalization factor; or ii) 1, wherein the normalization factor is a high percentile value (e.g., 90, 91, 92, 93, 94, 95, 96, 97, 98, or 99^{th} percentile) of the number of PET reads supporting any genomic complex (e.g., ASMC) in the cell population (e.g., the integrity index as determined by Formula 3). In some embodiments, the normalization factor is the 99^{th} percentile of the number of PET reads supporting any genomic complex (e.g., ASMC) in the cell population (e.g., as seen in Formula 3 and measured by the method of Example 2). Without wishing to be bound by theory, it may be advantageous to use a high percentile value of the number of PET reads of all genomic complexes (e.g., ASMCs), as opposed to the highest number of PET reads of all genomic complexes (e.g., ASMCs) observed in a cell population, to avoid the issue of very stable and/or omnipresent outlier genomic complexes (e.g., ASMCs). The number of PET reads supporting the presence of a given genomic complex (e.g., ASMC) in a cell population may be measured, e.g., by an experimental technique such as ChIA-PET. In some embodiments, ChIA-PET is used with regard to a particular genomic complex component of interest, e.g., a polypeptide component, e.g., a nucleating polypeptide, e.g., CTCF or YY1. In some embodiments, the integrity index of a target genomic complex (e.g., ASMC) i is determined by Formula 3:

$${IntInd}_{i} = min ( \frac{\begin{matrix}log 2 ( number of PETs supporting \\ genomic complex ( e . g . , ASMC ) i )\end{matrix}}{Normalization factor} , 1 )$$

In some embodiments, the integrity index of a particular genomic complex targeted for disruption as described herein is greater than about 0.25. In some embodiments, a genomic complex with an integrity index of greater than 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99, or more is targeted for disruption. Such targeted genomic complexes may be characterized as functional complexes (e.g., intact complexes). In some embodiments, genomic complexes with an integrity index in a range of about 0.3-0.99 are targeted for disruption. In some embodiments, genomic complexes with an integrity index in a range of about 0.3-0.99, 0.4-0.99, 0.5-0.99, 0.6-0.99, 0.7-0.99, 0.8-0.99, or 0.9-0.99 are targeted for disruption. In some embodiments, one or more genomic complexes with an integrity index in a range of about 0.3-0.9, 0.4-0.9, 0.5-0.9, 0.3-0.8, 0.4-0.8, 0.5-0.8, 0.6-0.8, 0.3-07, 0.4-0.7, 0.5-0.7, 0.6-0.7, or 0.5-0.6 are targeted for disruption.

In some embodiments, the integrity index of a target genomic complex (e.g., ASMC), e.g., targeted for modulation (e.g., disruption) by a method described herein, is a high integrity index, e.g., an integrity index of greater than or equal to 0.5 or greater than or equal to 0.75 (and optionally less than or equal to 1). Without wishing to be bound by theory, selecting and disrupting a genomic complex (e.g., ASMC) having a high integrity index, e.g., greater than about 0.5 (e.g., 0.5-1), reduces the probability of disrupting a genomic complex (e.g., ASMC) with such a low frequency of incidence that such targeting is unlikely to achieve significant impact on expression of a gene associated with said genomic complex (e.g., ASMC); in other words, selecting and disrupting a genomic complex (e.g., ASMC) having a high integrity index may make it more likely that the disruption has a significant effect on the expression of an associated gene. In some embodiments, the integrity index of a target genomic complex (e.g., ASMC), e.g., targeted for modulation (e.g., disruption) by a method described herein, is greater than or equal to 0.5. In some embodiments, a genomic complex (e.g., ASMC) has an integrity index of greater than or equal to 0.5, 0.55, 0.6, 0.65, 0.7, 0.75, 0.8, 0.85, 0.9, 0.91, 0.92, 0.93, 0.94, 0.95, 0.96, 0.97, 0.98, or 0.99 (and optionally, has an integrity index of less than or equal to 1, 0.99, 0.95, 0.9, 0.85, 0.8, 0.75, 0.7, 0.65, or 0.6) and is targeted for modulation (e.g., disruption). In some embodiments, a genomic complex (e.g., ASMC) has an integrity index of 0.5-1, 0.5-0.9, 0.5-0.8, 0.5-0.7, 0.5-0.6, 0.6-1, 0.6-0.9, 0.6-0.8, 0.6-0.7, 0.7-1, 0.7-0.9, 0.7-0.8, 0.8-1, 0.8-0.9, or 0.9-1 and is targeted for modulation (e.g., disruption).

In some embodiments, the integrity index of a target genomic complex (e.g., ASMC), e.g., targeted for modulation (e.g., disruption) by a method described herein, is an intermediate integrity index, e.g., an integrity index of greater than or equal to 0.25 and less than or equal to 0.75. Without wishing to be bound by theory, selecting and disrupting a genomic complex (e.g., ASMC) having an intermediate integrity index, e.g., greater than about 0.25 and less than or equal to 0.75, reduces the probability of: i) disrupting a genomic complex (e.g., ASMC) with such a low frequency of incidence that such targeting is unlikely to achieve significant impact on expression of a gene associated with said genomic complex (e.g., ASMC) and/or ii) attempting to disrupt a genomic complex (e.g., ASMC) whose incidence is so high (e.g., and interactions holding together said complex so strong and/or stable) that modulation (e.g., disruption) of the complex is difficult or unlikely. In other words, selecting and disrupting a genomic complex (e.g., ASMC) having an intermediate integrity index may make it more likely that the disruption has a significant effect on the expression of an associated gene. In some embodiments, the integrity index of a target genomic complex (e.g., ASMC), e.g., targeted for modulation (e.g., disruption) by a method described herein, is greater than or equal to 0.25 and less than or equal to 0.75. In some embodiments, a genomic complex (e.g., ASMC) has an integrity index of greater than or equal to 0.25, 0.3, 0.35, 0.4, 0.45, 0.5, 0.55, 0.6, 0.65, or 0.7 (and optionally, has an integrity index of less than or equal to 0.75, 0.7, 0.65, 0.6, 0.55, 0.5, 0.45, 0.4, 0.35, or 0.3) and is targeted for modulation (e.g., disruption). In some embodiments, a genomic complex (e.g., ASMC) has an integrity index of 0.25-0.75, 0.25-0.65, 0.25-0.55, 0.25-0.45, 0.25-0.35, 0.35-0.75, 0.35-0.65, 0.35-0.55, 0.35-0.45, 0.45-0.75, 0.45-0.65, 0.45-0.55, 0.55-0.75, 0.55-0.65, or 0.65-0.75 and is targeted for modulation (e.g., disruption).

In some embodiments, data points for determining the integrity index of a genomic complex (e.g., ASMC), e.g., by input into Formulas 2 or 3, are determined experimentally, for example using one or more assay(s) and/or analyses as described herein (see, e.g., doi-10.1038/nmeth.3999). In some embodiments, integrity indices may be determined by assessing methylation occupancy status via a ChIA pet and/or, ChIP (e.g., methylation anchor site occupancy as a proxy for genomic complex formation and/or integrity) analysis, e.g., in a cell population and optionally at a plurality of time points so that integrity index is assessed over time. In some embodiments, such analyses may be performed with respect to a single genomic complex (e.g., ASMC), a plurality of genomic complexes (e.g., ASMCs), or genome-wide, in order to determine, or inform determination of, an integrity index for a target genomic complex (e.g., ASMC) or plurality of target genomic complexes (e.g., ASMCs). In some embodiments, such analyses may be performed in more than one cell type, and an integrity index may be assigned to the particular genomic complex (e.g., ASMC) for each cell type.

In some embodiments, a target genomic complex (e.g., ASMC) is selected and/or identified as having an integrity index above or below a particular threshold and/or within a particular range. For example, in some embodiments, observation of a genomic complex (e.g., ASMC) having an integrity index above or below a particular threshold and/or within a particular range in a particular cell or cell type of interest may identify that genomic complex (e.g., ASMC) and associated gene as a candidate genomic complex (e.g., ASMC) for targeting with a method described herein. In some embodiments, the genomic analyses (e.g., used to determine the integrity index) such as methylation occupancy status via ChIA pet and/or, ChIP, are also used to determine a gene associated with the candidate genomic complex (e.g., ASMC). Determination or identification of an associated gene (e.g., a gene whose expression may be impacted by the presence and/or extent of the particular genomic complex (e.g., ASMC)) may contribute to identification and/or characterization of a candidate target genomic complex (e.g., ASMC) as a target genomic complex (e.g., ASMC). Among other things, the present disclosure teaches that identification and/or characterization of integrity index of a genomic complex (e.g., ASMC) can usefully determine a genomic complex (e.g., ASMC) that, when targeted with a modulating (e.g., disrupting) agent as described herein, are likely to impact biology of cells containing the genomic complex (e.g., ASMC).

### A ChIA-PET Method for Integrity Index

In some embodiments, an integrity index is determined by analyzing a ChIA-PET dataset, e.g., a nucleating polypeptide ChIA-PET dataset, e.g., a CTCF ChIA-PET dataset. Publicly available ChIA-PET datasets directed to different DNA-binding polypeptides (e.g., nucleating polypeptides) are known to those of skill in the art, as is software and methodology for processing said data (e.g., as taught by Li et al. ChIA-PET2: a versatile and flexible pipeline for ChIA-PET data analysis (2017). Nucleic Acids Research 45(1):e4). In some embodiments, a method, e.g., a pipeline, for analyzing ChIA-PET data comprises one or more of (e.g., all of): an alignment step; a step of making a BEDPE file (or similar file capable of annotating inter-chromosomal structural information in sequence) with unique paired end tags (PETs); a peak calling step; a PET clustering/loop calling step; and a loop significance calling and/or filtering step. In some embodiments, the method, e.g., pipeline, for analyzing ChIA-PET data further comprises applying the data generated in previous steps to Formula 3 to calculate the integrity index of one or more (e.g., each) genomic complex (e.g., ASMC) in the data.

In some embodiments, processing ChIA-PET data comprises an alignment step. In some embodiments, the alignment step comprises aligning paired raw sequencing reads independently for each lane of sequencing data, e.g., using Burrows-Wheeler Aligner (bwa). In some embodiments, the alignment step comprises converting bwa alignment data to a binary sequence storage format, e.g., a BAM file, e.g., using samtools (e.g., from Samtools Organization. Samtools (2019), https://github.com/samtools/samtools). In some embodiments, the alignment step comprises sorting aligned reads by read name, e.g., by using the Picard SortSam command, e.g., of Broad Institute. Picard (2019), https://broadinstitute.github.io/picard/. In some embodiments, the alignment step comprises the steps disclosed herein in the order performed in Examples 1 or 2.

In some embodiments, processing ChIA-PET data comprises a step of making a BEDPE file (or similar file capable of annotating inter-chromosomal structural information in sequence) with unique paired end tags (PETs). In some embodiments, the step of making a BEDPE file (or similar file capable of annotating inter-chromosomal structural information in sequence) with unique PETs comprises passing independently aligned and/or sorted binary sequence storage files (e.g., BAM files) to the buildBedpe command of ChIA-PET2 (e.g., with parameters mapq cutoff 30, threads 4, keep_seq 0) or similar command to produce a BEDPE file (or similar file capable of annotating inter-chromosomal structural information in sequence). In some embodiments, the step of making a BEDPE file (or similar file capable of annotating inter-chromosomal structural information in sequence) with unique PETs comprises combining BEDPE files from multiple lanes of sequencing data, e.g., using the Unix “cat” command or similar concatenation software. In some embodiments, the step of making a BEDPE file (or similar file capable of annotating inter-chromosomal structural information in sequence) with unique PETs comprises removing duplicate PETs from the BEDPE file(s), e.g., using the “rmdup” command from ChIA-PET2. In some embodiments, the step of making a BEDPE file (or similar file capable of annotating inter-chromosomal structural information in sequence) with unique PETs comprises the steps disclosed herein in the order performed in Examples 1 or 2.

In some embodiments, processing ChIA-PET data comprises a peak calling step. In some embodiments, the peak calling step comprises converting a BEDPE file (or similar file capable of annotating inter-chromosomal structural information in sequence) into a tags file, e.g., wherein the tags are sorted, e.g., using the Unix “sort” command or similar functionality. In some embodiments, the peak calling step comprises calling peaks (e.g., using the sorted tags file), e.g., using MACS2 or a tool with similar functionality. In some embodiments, the peak calling step comprises expanding peaks (e.g., by at least 100, 200, 300, 400, 500, 600, 700, 800, or 900 base pairs (and optionally no more than 1000, 900, 800, 700, 600, or 500 base pairs), e.g., by 500 base pairs) in either direction, e.g., using the bedtools “slopBed” command or a similar functionality. In some embodiments, the peak calling step comprises computing sequencing coverage (e.g., peak depth) at each peak, e.g., using the bedtools “coverageBed” or similar functionality. In some embodiments, the peak calling step comprises the steps disclosed herein in the order performed in Examples 1 or 2.

In some embodiments, processing ChIA-PET data comprises a PET clustering/loop calling step. In some embodiments, the PET clustering/loop calling step comprises processing a BEDPE file (or similar file capable of annotating inter-chromosomal structural information in sequence), e.g., with expanded peaks as described herein, and sequencing coverage (e.g., peak depth) data to create a BEDPE file (or similar file capable of annotating inter-chromosomal structural information in sequence) filtered for PETs between called peaks, e.g., using the “pairToBed” command of bedtools or similar functionality. In some embodiments, the PET clustering/loop calling step comprises clustering PETs by peak pairs, e.g., using the “bedpe2Interaction” command from ChIA-PET2 or similar functionality, e.g., generating lists (e.g., files) containing intra- and/or inter-chromosomal PET clusters. In some embodiments, a file contains one row per peak pair with the peak depth at each peak and number of PETs between that pair of peaks, representing an individual loop call. In some embodiments, PET clustering/loop calling step comprises the steps disclosed herein in the order performed in Examples 1 or 2.

In some embodiments, processing ChIA-PET data comprises a loop significance calling and/or filtering step. In some embodiments, the loop significance calling and/or filtering step comprises calculating loop significance, e.g., by computing p-value(s) and false discovery rate (FDR) q-value(s) for loops, e.g., loops identified in a previous loop calling step. In some embodiments, calculating loop significance comprises using the MICC algorithm (He et al., MICC: an R package for identifying chromatin interactions from ChIA-PET data (2015). Bioinformatics 31(23):3832-4) or a variant thereof, e.g., the MICC2.R script of ChIA-PET2. In some embodiments, the loop significance calling and/or filtering step comprises filtering the output of a MICC algorithm, e.g., to include only peaks that meet one or more thresholds. In some embodiments, the one or more (e.g., two) thresholds are chosen from: peaks with a FDR q-value of less than or equal to a reference value (e.g., an empirically defined reference value, e.g., either 0.05 or 0.1); or loops supported by a minimum number of PETs (e.g., an empirically defined minimum number of PETs, e.g., 2, 3, or 5). In some embodiments, the incorporation of thresholds is used to maintain consistency or comparability of the number of called loops across different experiments. In some embodiments, the thresholds and/or empirically defined values are chosen such that at least 5000, 10,000, 20,000, 30,000, 40,000, 50,000, 60,000, 70,000, 80,000, 90,000, or 100,000 significant loops are called (and optionally, no more than 200,000, 190,000, 180,000, 170,000, 160,000, 150,000, 140,000, 130,000, 120,000, 110,000, 100,000, 90,000, 80,000, or 70,000 significant loops are called). In some embodiments, the loop significance calling and/or filtering step comprises the steps disclosed herein in the order performed in Examples 1 or 2.

In some embodiments, processing ChIA-PET data comprises applying the called and/or filtered loop data to a formula for integrity index, e.g., as described herein. In some embodiments, the formula for integrity index is Formula 2. In some embodiments, the formula for integrity index is Formula 3.

### Specificity Index

The present disclosure is directed, in part, to methods of modulating, e.g., disrupting, a genomic complex (e.g., ASMC), wherein the genomic complex (e.g., ASMC) has or is identified as having a specificity index of a particular value or within a range of values. The specificity index is a value that is a quantitative representation of how common or unique a genomic complex (e.g., ASMC) is among a plurality of cell populations, e.g., across a target cell population and at least one reference cell population. The specificity index may be calculated, e.g., by Formula 1.

$${SpecInd}_{i} = \frac{\begin{matrix}\# of cell lines where genomic \\ complex ( e . g . , ASMC ) i is present\end{matrix}}{Total \# of cell lines}$$

In some embodiments, a cell population corresponds to a cell line (e.g., a cell line known to those of skill in the art). In some embodiments, a cell population corresponds to cells of a particular tissue, or cellular or developmental lineage. In some embodiments, a cell population correspons to cells of a particular phenotype (e.g., a disease or non-disease phenotype). In some embodiments, a cell population corresponds to cells at a particular time or developmental stage relative to a subject, e.g., hepatocytes from a juvenile human subject. Each of these delineated cell populations may be referred herein to as different cell types.

Without wishing to be bound by theory, it may be advantageous to target a genomic complex (e.g., ASMC) that is present in a target cell or cell type of interest and that has a low specificity index (e.g., less than 0.5). A low specificity index indicates that a genomic complex (e.g., ASMC) is present in fewer cell populations than a genomic complex having a high specificity index. Targeting a genomic complex (e.g., ASMC) with a low specificity index may cause fewer off-target effects in non-target cells by virtue of the target genomic complex not being present in as many non-target cells. For example, it may be advantageous to target a genomic complex (e.g., ASMC) present only in a cell type of interest for the purposes of altering expression of a target gene associated with the target genomic complex, because it is less likely (e.g., not likely) that targeting said genomic complex would affect expression of the target gene in other cell types not comprising the target genomic complex.

It will be apparent to one of skill in the art that the value of the specificity index of a given genomic complex (e.g., ASMC) depends upon the number of cell populations being referenced. For example, if a target genomic complex (e.g., ASMC) is present in a target cell population and also present in 9 other selected reference cell populations, e.g., 9 non-target cell populations, then the specificity index of the target genomic complex (e.g., ASMC) is 0.1. In some embodiments, reference cell populations are selected from non-target cell types, e.g., cell types in which modulation (e.g., disruption) of a target genomic complex (e.g., ASMC) is not intended. In some embodiments, reference cell populations are selected from non-target cell types that are likely to be exposed to a modulating agent (e.g., disrupting agent) upon administration to a subject (e.g., for the purposes of modulating (e.g., disrupting) a target genomic complex (e.g., ASMC)). In some embodiments, reference cell populations are selected from cell types for which inter-/intra-chromosomal interaction data (e.g., ChIA-PET data) is available (e.g., from the Encode Consortium (https//www.encodeproject.org/)), e.g., inter-/intra-chromosomal interaction data at the target genomic complex (e.g., ASMC). In some embodiments, reference cell populations are selected from all cell types for which inter-/intra-chromosomal interaction data (e.g., ChIA-PET data) is available (e.g., from the Encode Consortium (https//www.encodeproject.org/) as of Sep. 23, 2019), e.g., inter-/intra-chromosomal interaction data at the target genomic complex (e.g., ASMC).

In some embodiments, the specificity index is determined using at least 2, 3, 4, 5, 6, 7, 8, 9, 10, 11, 12, 13, 14, 15, 16, 17, 18, 19, or 20 total cell types (e.g., at least 1, 2, 3, 4, 5, 6, 7, 8, 9, 10, 11, 12, 13, 14, 15, 16, 17, 18, or 19 reference cell populations in addition to a target cell population). Optionally, the specificity index is determined using no more than 50, 40, 30, 20, 15, or 10 total cell types (e.g., no more than 49, 39, 29, 19, 14, or 9 reference cell populations in addition to a target cell population). In some embodiments, the specificity index is determined using one or more (e.g., all) of the cell types of Table 2. In some embodiments, a target cell population is selected from stem cells, progenitor cells, differentiated and/or mature cells, post-mitotic cells, e.g., liver, skin, brain, caudate and/or putamen nuclei, hepatocytes, fibroblasts, CD34+ cells, CD3+ cells. In some embodiments, reference cell populations are selected from stem cells, progenitor cells, differentiated and/or mature cells, post-mitotic cells, e.g., liver, skin, brain, caudate and/or putamen nuclei, hepatocytes, fibroblasts, CD34+ cells, CD3+ cells.

In some embodiments, the specificity index of a target genomic complex (e.g., ASMC), e.g., targeted for modulation (e.g., disruption) by a method described herein, is less than or equal to 0.5. In some embodiments, a genomic complex (e.g., ASMC) has a specificity index of less than 0.5, 0.45, 0.4, 0.35, 0.3, 0.25, 0.2, 0.15, 0.1, or 0.05 (and optionally, has a specificity index of greater than or equal to 0.01, 0.05, 0.1, 0.15, 0.2, 0.25, 0.3, 0.35, 0.4, or 0.45) and is targeted for modulation (e.g., disruption). In some embodiments, a genomic complex (e.g., ASMC) has an integrity index of 0.01-0.5, 0.01-0.45, 0.01-0.4, 0.01-0.35, 0.01-0.3, 0.01-0.25, 0.01-0.2, 0.01-0.15, 0.01-0.1, 0.01-0.05, 0.05-0.5, 0.05-0.45, 0.05-0.4, 0.05-0.35, 0.05-0.3, 0.05-0.25, 0.05-0.2, 0.05-0.15, 0.05-0.1, 0.1-0.5, 0.1-0.45, 0.1-0.4, 0.1-0.35, 0.1-0.3, 0.1-0.25, 0.1-0.2, 0.1-0.15, 0.15-0.5, 0.15-0.45, 0.15-0.4, 0.15-0.35, 0.15-0.3, 0.15-0.25, 0.15-0.2, 0.2-0.5, 0.2-0.45, 0.2-0.4, 0.2-0.35, 0.2-0.3, 0.2-0.25, 0.25-0.5, 0.25-0.45, 0.25-0.4, 0.25-0.35, 0.25-0.3, 0.3-0.5, 0.3-0.45, 0.3-0.4, 0.3-0.35, 0.35-0.5, 0.35-0.45, 0.35-0.4, 0.4-0.5, 0.4-0.45, or 0.45-0.5 and is targeted for modulation (e.g., disruption).

The present disclosure is directed, in part, to methods of modulating, e.g., disrupting, a genomic complex (e.g., ASMC), wherein the genomic complex (e.g., ASMC): is present or is identified as being present in a target cell type; and is present or is identified as being present in less than a threshold number of reference cell populations.

In some embodiments, reference cell types are selected from non-target cell types, e.g., cell types in which modulation (e.g., disruption) of a target genomic complex (e.g., ASMC) is not intended. In some embodiments, reference cell populations are selected from non-target cell types that are likely to be exposed to a modulating agent (e.g., disrupting agent) upon administration to a subject (e.g., for the purposes of modulating (e.g., disrupting) a target genomic complex (e.g., ASMC)). In some embodiments, reference cell populations are selected from cell types for which inter-/intra-chromosomal interaction data (e.g., ChIA-PET data) is available (e.g., from the Encode Consortium (https//www.encodeproject.org/)), e.g., inter-/intra-chromosomal interaction data at the target genomic complex (e.g., ASMC). In some embodiments, reference cell populations are selected from all cell types for which inter-/intra-chromosomal interaction data (e.g., ChIA-PET data) is available (e.g., from the Encode Consortium (https//www.encodeproject.org/) as of Sep. 23, 2019), e.g., inter-/intra-chromosomal interaction data at the target genomic complex (e.g., ASMC).

### Methods for Specificity Index

In some embodiments, a specificity index is determined by analyzing a ChIA-PET dataset, e.g., a nucleating polypeptide ChIA-PET dataset, e.g., a CTCF ChIA-PET dataset. Publicly available ChIA-PET datasets directed to different DNA-binding polypeptides (e.g., nucleating polypeptides) are known to those of skill in the art, as is software and methodology for processing said data (e.g., as taught by Li et al. ChIA-PET2: a versatile and flexible pipeline for ChIA-PET data analysis (2017). Nucleic Acids Research 45(1):e4). In some embodiments, a method, e.g., a pipeline, for analyzing ChIA-PET data comprises one or more of (e.g., all of): an alignment step; a step of making a BEDPE file (or similar file capable of annotating inter-chromosomal structural information in sequence) with unique paired end tags (PETs); a peak calling step; a PET clustering/loop calling step; and a loop significance calling and/or filtering step. The discussion of said steps in the context of integrity indices also applies to preparation of the data for calculating specificity indices. In some embodiments, the method, e.g., pipeline, for analyzing ChIA-PET data further comprises applying the data generated in previous steps to Formula 1 to calculate the specificity indices of one or more (e.g., each) genomic complex (e.g., ASMC) in the data.

In some embodiments, a specificity index is determined by analyzing a 4C dataset, e.g., a 4C-seq dataset, e.g., not requiring a specific immunoprecipitation step. 4C-seq data can be processed using software and methodology known to those of skill in the art, e.g., 4Cseqpipe processing pipeline methodologies. In some embodiments, the output of said software and methodologies is a list of significant loops. In some embodiments, said list of significant loops may be used to calculate a specificity index, e.g., using Formula 1.

### Modulating (e.g., Disrupting) Agents

As described herein, the present disclosure provides technologies for modulating (e.g., disrupting) a genomic complex (e.g., ASMC) by contacting a system in which such complexes have formed or would otherwise be expected to form with a modulating (e.g., disrupting) agent as described herein. In some embodiments, the extent of genomic complex (e.g., ASMC) formation and/or maintenance (e.g., number of complexes in a system at a given moment in time, or over a period of time) is altered (e.g., reduced) by the presence of the modulating agent, e.g., disrupting agent, as compared with the extent observed in the absence of the modulating (e.g., disrupting) agent. In some embodiments, modulating (e.g., disrupting) agents bind to and/or interact with one or more target genomic complexes (e.g., ASMCs) based on relative abundance, quantified by integrity index.

In general, a modulating (e.g., disrupting) agent as described herein interacts with its target component of a genomic complex (e.g., ASMC). In some embodiments, modulating (e.g., disrupting) agents do not target genomic sequence elements. In some embodiments, targeting may include targeting of one or more genomic sequence elements, for example, in addition to targeting one or more other components as described herein. In some embodiments, modulating (e.g., disrupting) agents may target one or more genomic sequence elements, which genomic sequence element(s) is/are distinct from an anchor sequence. For example, in order to modulate a particular genomic complex (e.g., ASMC), a modulating (e.g., disrupting) agent may target a genomic sequence element that is or comprises a binding site of a transcription factor that is part of the genomic complex.

In some embodiments, a modulating (e.g., disrupting) agent modulates (e.g., disrupts) one or more aspects of a genomic complex (e.g., ASMC). In some embodiments, modulation (e.g., disruption) is or comprises modulation (e.g., disruption) of a topological structure of a genomic complex (e.g., ASMC). In some embodiments, modulation (e.g., disruption) of a topological structure of a genomic complex results in altered (e.g., decreased or increased) expression of a given target gene. In some embodiments, no detectable modulation (e.g., disruption) of a topological structure is observed, but altered expression of a given target gene is nonetheless observed. In some embodiments, modulation (e.g., disruption) is or comprises binding to a component of the genomic complex (e.g., ASMC). Binding may result in sequestering of the component or degradation of the component (e.g., by an enzyme of the cell); in either exemplary case, the level of the component, is altered, e.g., decreased, and the level or occupancy of the genomic complex (e.g., ASMC), e.g., at a target gene, is thereby altered.

Those skilled in the art will appreciate that, in certain instances, two or more genomic complexes (e.g., ASMCs) may compete with each other with respect to a particular genomic region or particular genomic location. In some embodiments, disruption of one (a “first”) genomic complex (e.g., ASMC) may be achieved by stabilization of one or more other genomic complexes (e.g., ASMCs) that represent alternative (relative to the first genomic complex) structures available to the particular genomic region or location. In some embodiments, stabilization of one (a “first) genomic complex (e.g., ASMC) may be achieved by disruption of one or more other genomic complexes (e.g., ASMCs) that represent alternative (relative to the first genomic complex) structures available to the particular genomic region or location. Thus, in some embodiments, disruption or stabilization of a genomic complex (e.g., ASMC) of interest may be achieved by targeting one or more competing genomic complexes for stabilization or disruption respectively (optionally without also providing a modulating agent that disrupts or stabilizes the genomic complex (e.g., ASMC) of interest).

In some embodiments, a particular genomic complex (e.g., ASMC) of interest may, in a particular cell, cell type, and/or developmental stage, be characterized by an integrity index outside of that preferred for targeting as described herein. In some embodiments, one or more steps can be taken to adjust the integrity index for that genomic complex (e.g., ASMC) to render it a more desirable target for modulation (e.g., disruption). In some embodiments, one or more steps can be taken to adjust the integrity index for that genomic complex (e.g., ASMC) so as to render it further disrupted (e.g. further decrease an integrity index of a particular genomic complex, e.g. further decrease the incidence of a particular genomic complex).

In some embodiments, interaction of a modulating (e.g., disrupting) agent and a target component of a given genomic complex results in alteration of gene expression. In some embodiments, alteration may be or comprise a change (e.g., increase or decrease in expression) relative to gene expression in the absence of a modulating (e.g., disrupting) agent.

In some embodiments, a target genomic complex is targeted based upon its integrity index. In some embodiments, integrity indices of particular genomic complexes (e.g., ASMCs) may differ between particular cell types. In some embodiments, integrity indices of particular genomic complexes (e.g., ASMCs) may differ between particular timepoints and/or developmental stages of one or more cells.

A modulating agent (e.g., disrupting agent) may bind its target component of a genomic complex (e.g., ASMC) and alter formation of the genomic complex (e.g., by altering affinity of the targeted component to one or more other complex components, e.g., by at least 10%, 15%, 20%, 25%, 30%, 35%, 40%, 45%, 50%, 55%, 60%, 65%, 70%, 75%, 80%, 85%, 90%, 95%, or more). Alternatively or additionally, in some embodiments, binding by a modulating agent alters topology of genomic DNA impacted by a genomic complex, e.g., at least 10%, 15%, 20%, 25%, 30%, 35%, 40%, 45%, 50%, 55%, 60%, 65%, 70%, 75%, 80%, 85%, 90%, 95%, or more. In some embodiments, a modulating agent (e.g., disrupting agent) alters expression of a gene associated with a targeted genomic complex (e.g., ASMC) by at least 10%, 15%, 20%, 25%, 30%, 35%, 40%, 45%, 50%, 55%, 60%, 65%, 70%, 75%, 80%, 85%, 90%, 95%, or more. Changes in genomic complex formation, affinity of targeted components for other complex components, and/or changes in topology of genomic DNA impacted by a genomic complex may be evaluated, for example, using HiChIP, ChIAPET, 4C, or 3C, e.g., HiChIP.

In some embodiments, a modulating agent (e.g., disrupting agent) alters (e.g., decrease) the integrity index of a targeted genomic complex (e.g., ASMC) by at least 10%, 15%, 20%, 25%, 30%, 35%, 40%, 45%, 50%, 55%, 60%, 65%, 70%, 75%, 80%, 85%, 90%, 95%, or more. In some embodiments, a modulating agent (e.g., disrupting agent) decreases the integrity index of a targeted genomic complex (e.g., ASMC) by at least 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, or 0.9 (and optionally less than 1, 0.95, 0.9, 0.85, 0.8, 0.75, 0.7, 0.65, 0.6, 0.55, or 0.5).

A modulating (e.g., disrupting) agent as described herein comprises a targeting moiety. In some embodiments, a targeting moiety binds to a target genomic complex (e.g., ASMC) component. In some embodiments, interaction between a targeting moiety and its targeted component interferes with one or more other interactions that the targeted component would otherwise make. In some embodiments, a modulating agent (e.g., disrupting agent) physically interferes with formation and/or maintenance of a genomic complex (e.g., ASMC), e.g., via the binding of the targeting moiety to its target genomic complex component.

In some embodiments, the modulating (e.g., disrupting) agent is a targeting moiety (e.g., the targeting interaction achieves the modulation, e.g., disruption, effect). In some embodiments, a modulating (e.g., disrupting) agent comprises a targeting moiety that interacts with its target component of a genomic complex (e.g., ASMC) and also comprises a separable or separate effector moiety (e.g., an effector moiety that independently affects the level, stability, or formation of the genomic complex (e.g., ASMC) level), and/or one or more additional moieties. For example, in some embodiments, a modulating (e.g., disrupting) agent, as provided herein, comprises a targeting moiety that binds its targeted component, and is operably linked to an effector moiety that modulates formation of one or more particular genomic complexes (e.g., ASMCs) in which the targeted component participates.

In some embodiments, a modulating (e.g., disrupting) agent is complex-specific. That is, in some embodiments, a targeting moiety binds specifically to its target component in one or more target genomic complexes (e.g., within a cell) and not to non-targeted genomic complexes (e.g., within the same cell). In some embodiments, a modulating (e.g., disrupting) agent specifically targets a genomic complex that is present in only certain cell types and/or only at certain developmental stages or times. In some embodiments, presence of a target genomic complex is determined based on integrity index scores.

In some embodiments, the present disclosure provides a modulating agent (e.g., disrupting agent) comprising an effector moiety which enhances the modulation (e.g., disruption) of a genomic complex (e.g., ASMC) in addition to or separate from any effect a targeting moiety may have the genomic complex (e.g., ASMC). In some embodiments, the effector moiety disrupts (e.g., inhibits/decreases formation and/or stability of) the genomic complex (e.g., ASMC). In some embodiments, the present disclosure provides a modulating agent (e.g., disrupting agent) comprising an effector moiety which enhances the modulation of the expression, e.g., decrease or increase of expression, of a target gene (e.g., a target gene associated with a genomic complex (e.g., ASMC)) in addition to or separate from any effect a targeting moiety may have on expression of the target gene. In some embodiments, the effector moiety decreases expression of the target gene. In some embodiments, the effector moiety does not bind to a genomic complex (e.g., ASMC) component (e.g., does not bind to the genomic complex component which the targeting moiety binds to).

As described in more detail below, a modulating agent, e.g., disrupting agent, (and/or any of a targeting moiety, effector moiety, and/or other moiety) may be or comprise a polypeptide, e.g., a protein or protein fragment, an antibody or antibody fragment (e.g., an antigen-binding fragment, a fusion molecule, etc), an oligonucleotide, a peptide nucleic acid, a small molecule, etc. and/or may include one or more non-natural residues or other structures. In some embodiments, a modulating agent may be or include an aptamer and/or a pharmacoagent, particularly one with poor pharmacokinetics as described herein.

A modulating agent may be or comprise a fusion molecule. In some embodiments, a fusion molecule comprises a targeting moiety and an effector moiety which are covalently connected to one another.

In some embodiments, a modulating agent (e.g., disrupting agent), e.g., the targeting moiety of a fusion molecule, comprises no more than 100, 90, 80, 70, 60, 50, 40, 30, or 20 nucleotides (and optionally at least 10, 20, 30, 40, 50, 60, 70, 80, or 90 nucleotides). In some embodiments, a modulating agent (e.g., disrupting agent), e.g., the effector moiety of a fusion molecule, comprises no more than 2000, 1900, 1800, 1700, 1600, 1500, 1400, 1300, 1200, 1100, 1000, 900, 800, 700, 600, 500, 400, 300, 200, or 100 amino acids (and optionally at least 50, 100, 200, 300, 400, 500, 600, 700, 800, 900, 1000, 1100, 1200, 1300, 1400, 1500, 1600, 1700, 1800, or 1900 amino acids). In some embodiments, a modulating agent (e.g., disrupting agent), e.g., the effector moiety of a fusion molecule, comprises 100-2000, 100-1900, 100-1800, 100-1700, 100-1600, 100-1500, 100-1400, 100-1300, 100-1200, 100-1100, 100-1000, 100-900, 100-800, 100-700, 100-600, 100-500, 100-400, 100-300, 100-200, 200-2000, 200-1900, 200-1800, 200-1700, 200-1600, 200-1500, 200-1400, 200-1300, 200-1200, 200-1100, 200-1000, 200-900, 200-800, 200-700, 200-600, 200-500, 200-400, 200-300, 300-2000, 300-1900, 300-1800, 300-1700, 300-1600, 300-1500, 300-1400, 300-1300, 300-1200, 300-1100, 300-1000, 300-900, 300-800, 300-700, 300-600, 300-500, 300-400, 400-2000, 400-1900, 400-1800, 400-1700, 400-1600, 400-1500, 400-1400, 400-1300, 400-1200, 400-1100, 400-1000, 400-900, 400-800, 400-700, 400-600, 400-500, 500-2000, 500-1900, 500-1800, 500-1700, 500-1600, 500-1500, 500-1400, 500-1300, 500-1200, 500-1100, 500-1000, 500-900, 500-800, 500-700, 500-600, 600-2000, 600-1900, 600-1800, 600-1700, 600-1600, 600-1500, 600-1400, 600-1300, 600-1200, 600-1100, 600-1000, 600-900, 600-800, 600-700, 700-2000, 700-1900, 700-1800, 700-1700, 700-1600, 700-1500, 700-1400, 700-1300, 700-1200, 700-1100, 700-1000, 700-900, 700-800, 800-2000, 800-1900, 800-1800, 800-1700, 800-1600, 800-1500, 800-1400, 800-1300, 800-1200, 800-1100, 800-1000, 800-900, 900-2000, 900-1900, 900-1800, 900-1700, 900-1600, 900-1500, 900-1400, 900-1300, 900-1200, 900-1100, 900-1000, 1000-2000, 1000-1900, 1000-1800, 1000-1700, 1000-1600, 1000-1500, 1000-1400, 1000-1300, 1000-1200, 1000-1100, 1100-2000, 1100-1900, 1100-1800, 1100-1700, 1100-1600, 1100-1500, 1100-1400, 1100-1300, 1100-1200, 1200-2000, 1200-1900, 1200-1800, 1200-1700, 1200-1600, 1200-1500, 1200-1400, 1200-1300, 1300-2000, 1300-1900, 1300-1800, 1300-1700, 1300-1600, 1300-1500, 1300-1400, 1400-2000, 1400-1900, 1400-1800, 1400-1700, 1400-1600, 1400-1500, 1500-2000, 1500-1900, 1500-1800, 1500-1700, 1500-1600, 1600-2000, 1600-1900, 1600-1800, 1600-1700, 1700-2000, 1700-1900, 1700-1800, 1800-2000, 1800-1900, or 1900-2000 amino acids.

A modulating agent, e.g., disrupting agent, may comprise nucleic acid, e.g., one or more nucleic acids. The term “nucleic acid” refers to any compound that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

In some embodiments, a targeting moiety comprises or is nucleic acid. In some embodiments, an effector moiety comprises or is nucleic acid. In some embodiments, a nucleic acid that may be included in a nucleic acid moiety or entity as described herein, may be or comprise DNA, RNA, and/or an artificial or synthetic nucleic acid or nucleic acid analog or mimic. For example, in some embodiments, a nucleic acid included in a nucleic acid moiety as described herein may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. In some embodiments, a nucleic acid may include one or more residues that is not a naturally-occurring DNA or RNA residue, may include one or more linkages that is/are not phosphodiester bonds (e.g., that may be, for example, phosphorothioate bonds, etc), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

In some embodiments, nucleic acids may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Some examples of nucleic acids include, but are not limited to, a nucleic acid that hybridizes to an endogenous gene (e.g., gRNA or antisense ssDNA as described herein elsewhere), a nucleic acid that hybridizes to an exogenous nucleic acid such as a viral DNA or RNA, nucleic acid that hybridizes to an RNA, a nucleic acid that interferes with gene transcription, a nucleic acid that interferes with RNA translation, a nucleic acid that stabilizes RNA or destabilizes RNA such as through targeting for degradation, a nucleic acid that interferes with a DNA or RNA binding factor through interference of its expression or its function, a nucleic acid that is linked to a intracellular protein or protein complex and modulates its function, etc.

The present disclosure contemplates modulating agents, e.g., disrupting agents, comprising RNA therapeutics (e.g., modified RNAs) as useful components of provided compositions as described herein. For example, in some embodiments, a modified mRNA encoding a protein of interest may be linked to a polypeptide described herein and expressed in vivo in a subject.

In some embodiments, a modulating agent, e.g., disrupting agent, comprises one or more nucleoside analogs. In some embodiments, a nucleic acid sequence may include in addition or as an alternative to one or more natural nucleosides, e.g., purines or pyrimidines, e.g., adenine, cytosine, guanine, thymine and uracil, one or more nucleoside analogs. In some embodiments, a nucleic acid sequence includes one or more nucleoside analogs. A nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

In some embodiments, a modulating agent, e.g., disrupting agent, comprises a nucleic acid sequence that encodes a gene expression product.

In some embodiments, a targeting moiety comprises a nucleic acid that does not encode a gene expression product. For example, a targeting moiety may comprise an oligonucleotide that hybridizes to a ncRNA, e.g., an eRNA. For example, in some embodiments, a sequence of an oligonucleotide comprises a complement of a target eRNA, or has a sequence that is at least 80%, at least 85%, at least 90%, at least 95%, at least 97%, at least 98%, at least 99% identical to the complement of a target eRNA.

A nucleic acid sequence suitable for use in a modulating agent, e.g., disrupting agent, may include, but is not limited to, DNA, RNA, modified oligonucleotides (e.g., chemical modifications, such as modifications that alter backbone linkages, sugar molecules, and/or nucleic acid bases), and artificial nucleic acids. In some embodiments, a nucleic acid sequence includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules.

In some embodiments, a nucleic acid sequence suitable for use in a modulating agent, e.g., disrupting agent, has a length from about 15-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 215-190, 20-190, 30-190, 40-190, 50-190, 60-190, 70-190, 80-190, 90-190, 100-190, 110-190, 120-190, 130-190, 140-190, 150-190, 160-190, 170-190, 180-190, 15-180, 20-180, 30-180, 40-180, 50-180, 60-180, 70-180, 80-180, 90-180, 100-180, 110-180, 120-180, 130-180, 140-180, 150-180, 160-180, 170-180, 15-170, 20-170, 30-170, 40-170, 50-170, 60-170, 70-170, 80-170, 90-170, 100-170, 110-170, 120-170, 130-170, 140-170, 150-170, 160-170, 15-160, 20-160, 30-160, 40-160, 50-160, 60-160, 70-160, 80-160, 90-160, 100-160, 110-160, 120-160, 130-160, 140-160, 150-160, 215-150, 20-150, 30-150, 40-150, 50-150, 60-150, 70-150, 80-150, 90-150, 100-150, 110-150, 120-150, 130-150, 140-150, 15-140, 20-140, 30-140, 40-140, 50-140, 60-140, 70-140, 80-140, 90-140, 100-140, 110-140, 120-140, 130-140, 15-130, 20-130, 30-130, 40-130, 50-130, 60-130, 70-130, 80-130, 90-130, 100-130, 110-130, 120-130, 215-120, 20-120, 30-120, 40-120, 50-120, 60-120, 70-120, 80-120, 90-120, 100-120, 110-120, 15-110, 20-110, 30-110, 40-110, 50-110, 60-110, 70-110, 80-110, 90-110, 100-110, 15-100, 20-100, 30-100, 40-100, 50-100, 60-100, 70-100, 80-100, 90-100, 15-90, 20-90, 30-90, 40-90, 50-90, 60-90, 70-90, 80-90, 15-80, 20-80, 30-80, 40-80, 50-80, 60-80, 70-80, 15-70, 20-70, 30-70, 40-70, 50-70, 60-70, 15-60, 20-60, 30-60, 40-60, 50-60, 15-50, 20-50, 30-50, 40-50, 15-40, 20-40, 30-40, 15-30, 20-30, or 15-20 nucleotides, or any range therebetween.

In some embodiments, a nucleic acid (e.g., a nucleic acid encoding a modulating agent, e.g., disrupting agent, or a nucleic acid that is comprised in a modulating agent, e.g., disrupting agent) may comprise operably linked sequences. The term “operably linked” when referring to nucleic acid sequences describes a relationship between a first nucleic acid sequence and a second nucleic acid sequence wherein the first nucleic acid sequence can affect the second nucleic acid sequence, e.g., by being co-expressed together, e.g., as a fusion gene, and/or by affecting transcription, epigenetic modification, and/or chromosomal topology. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other. In an embodiment, a promoter or enhancer sequence that is operably linked to a sequence encoding a protein can promote the transcription of the sequence encoding a protein, e.g., in a cell or cell free system capable of performing transcription. In an embodiment, a first nucleic acid sequence encoding a protein or fragment of a protein that is operably linked to a second nucleic acid sequence encoding a second protein or second fragment of a protein are expressed together, e.g., the first and second nucleic acid sequences comprise a fusion gene and are transcribed and translated together to produce a fusion protein.

### Targeting Moiety

In some embodiments, a modulating agent, e.g., disrupting agent, is or comprises a targeting moiety. In some embodiments, a targeting moiety targets, e.g., binds, a component of a genomic complex (e.g., ASMC). The target of a targeting moiety may be referred to as its targeted component. A targeted component may be any genomic complex (e.g., ASMC) component, including but not limited to a genomic sequence element (e.g., promoter, enhancer, anchor sequence, gene (e.g., exon, intron, or UTR encoding sequence)), a polypeptide component (e.g., a nucleating polypeptide or transcription factor), or a non-genomic nucleic acid component (e.g., a ncRNA, e.g., an eRNA).

In some embodiments, interaction between a targeting moiety and its targeted component interferes with one or more other interactions that the targeted component would otherwise make. In some embodiments, binding of a targeting moiety to a targeted component prevents the targeted component from interacting with another transcription factor, genomic complex component, or genomic sequence element. In some embodiments, binding of a targeting moiety to a targeted component decreases binding affinity of the targeted component for another transcription factor, genomic complex component, or genomic sequence element. In some embodiments, K_{D} of a targeted component for another transcription factor, genomic complex component, or genomic sequence element increases by at least 1.05× (i.e., 1.05 times), 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20×, 50×, or 100× (and optionally no more than 20×, 10×, 9×, 8×, 7×, 6×, 5×, 4×, 3×, 2×, 1.9×, 1.8×, 1.7×, 1.6×, 1.5×, 1.4×, 1.3×, 1.2×, or 1.1×) in presence of a modulating agent, e.g., disrupting agent, comprising the targeting moiety than in the absence of the modulating agent, e.g., disrupting agent, comprising the targeting moiety. Changes in K_{D} of a targeted component for another transcription factor, genomic complex component, or genomic sequence element may be evaluated, for example, using ChIP-Seq or ChIP-qPCR.

In some embodiments, binding of a targeting moiety to a targeted component alters, e.g., decreases, the level of a genomic complex (e.g., ASMC) comprising the targeted component. In some embodiments, the level of a genomic complex (e.g., ASMC) comprising the targeted component decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the targeting moiety relative to the absence of said modulating agent. In some embodiments, binding of a targeting moiety to a targeted component alters, e.g., decreases, occupancy of the genomic complex (e.g., ASMC) at a genomic sequence element (e.g., a target gene, or a transcriptional control sequence operably linked thereto). In some embodiments, occupancy decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the targeting moiety relative to the absence of said modulating agent. Changes in genomic complex level and/or occupancy may be evaluated, for example, using HiChIP, ChIAPET, 4C, or 3C, e.g., HiChIP.

In some embodiments, binding of a targeting moiety to a targeted component alters, e.g., decreases, the occupancy of the genomic complex (e.g., ASMC) at a genomic sequence element (e.g., a gene, promoter, or enhancer, e.g., associated with the genomic or transcription complex). In some embodiments, binding of a targeting moiety to a targeted component decreases occupancy of the genomic complex (e.g., ASMC) at a genomic sequence element by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the targeting moiety relative to the absence of said modulating agent. In some embodiments, occupancy refers to the frequency with which an element can be found associated with another element, e.g., as determined by HiC, ChIP, immunoprecipitation, or other association measuring assays known in the art. In some embodiments, occupancy can be determined using integrity index (e.g., a change in integrity index may correspond to a change in occupancy).

In some embodiments, binding of a targeting moiety to a targeted component alters, e.g., decreases the occupancy of the targeted component in/at the genomic complex (e.g., ASMC). In some embodiments, binding of a targeting moiety to a targeted component decreases occupancy of the targeted component in/at the genomic complex (e.g., ASMC) by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the targeting moiety relative to the absence of said modulating agent.

In some embodiments, binding of a targeting moiety to a targeted component alters, e.g., decreases, the expression of a target gene associated with the genomic complex (e.g., ASMC) comprising the targeted component. In some embodiments, the expression of the target gene decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the targeting moiety relative to the absence of said modulating agent.

In some embodiments, a targeting moiety targets a polypeptide component of a genomic complex (e.g., ASMC). In some embodiments, said targeting moiety is or comprises a polypeptide (e.g., an antibody or antigen binding fragment thereof) that specifically binds with the target polypeptide component.

In some embodiments, a targeting moiety is or comprises a nucleic acid (e.g., an oligonucleotide (e.g., a gRNA, siRNA, etc.) which, in some embodiments, may contain one or more modified residues, linkages, or other features), a polypeptide (e.g., a protein, a protein fragment, an antibody, an antibody fragment (e.g., an antigen-binding fragment), or both. In some embodiments, the targeting moiety may include one or more modified residues, linkages, or other features), peptide nucleic acid, small molecule, etc.

In some embodiments, a targeting moiety is designed and/or administered so that it specifically interacts with a particular genomic complex (e.g., ASMC) relative to other genomic complexes (e.g., ASMCs) that may be present in the same system (e.g., cell, tissue, etc.). In some embodiments, a targeting moiety comprises a nucleic acid sequence complementary to a targeted component, e.g., a genomic sequence element or non-genomic nucleic acid component, in a genomic complex (e.g., ASMC). In some embodiments, a targeting moiety comprises a nucleic acid sequence that is complementary to at least 10%, 15%, 20%, 25%, 30%, 35%, 40%, 45%, 50%, 55%, 60%, 65%, 70%, 75%, 80%, 85%, 90%, 95%, or more of a targeted component, e.g., a genomic sequence element or non-genomic nucleic acid component, in a genomic complex (e.g., ASMC).

In some embodiments, a targeting moiety may be or comprise a CRISPR/Cas molecule, a TAL effector molecule, a Zn finger molecule, or a nucleic acid molecule.

CRISPR/Cas Molecules

In some embodiments, a targeting moiety is or comprises a CRISPR/Cas molecule. A CRISPR/Cas molecule comprises a protein involved in the clustered regulatory interspaced short palindromic repeat (CRISPR) system, e.g., a Cas protein, and optionally a guide RNA, e.g., single guide RNA (sgRNA).

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. For example, in a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by RNase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-targeting moiety includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments, the Cas protein is modified to deactivate the nuclease, e.g., nuclease-deficient Cas9.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas9 binding to a DNA sequence may interfere with transcription at that site by steric hindrance. In some embodiments, a targeting moiety is or comprises a catalytically inactive Cas9, e.g., dCas9. Many catalytically inactive Cas9 proteins are known in the art. In some embodiments, dCas9 comprises mutations in each endonuclease domain of the Cas protein, e.g., D10A and H840A mutations.

In some embodiments, a targeting moiety may comprise a Cas molecule comprising or linked (e.g., covalently) to a gRNA. A gRNA is a short synthetic RNA composed of a “scaffold” sequence necessary for Cas-protein binding and a user-defined ˜20 nucleotide targeting sequence for a genomic target. In practice, guide RNA sequences are generally designed to have a length of between 17-24 nucleotides (e.g., 19, 20, or 21 nucleotides) and be complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective for use with Cas proteins; see, for example, Hendel et al. (2015) Nature Biotechnol., 985-991.

In some embodiments, a gRNA comprises a nucleic acid sequence that is complementary to a DNA sequence associated with a target gene. In some embodiments, the DNA sequence is, comprises, or overlaps an expression control element that is operably linked to the target gene. In some embodiments, a gRNA comprises a nucleic acid sequence that is at least 90, 95, 99, or 100% complementary to a DNA sequence associated with a target gene. In some embodiments, a gRNA for use with a targeting moiety that comprises a Cas molecule is an sgRNA.

TAL Effector Molecules

In some embodiments, a targeting moiety is or comprises a TAL effector molecule. A TAL effector molecule, e.g., a TAL effector molecule that specifically binds a DNA sequence, comprises a plurality of TAL effector domains or fragments thereof, and optionally one or more additional portions of naturally occurring TAL effectors (e.g., N- and/or C-terminal of the plurality of TAL effector domains).

TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector feature a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 1 listing exemplary repeat variable diresidues (RVD) and their correspondence to nucleic acid base targets.

TABLE 1
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of the TAL effector molecule of the present invention may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicola strain BLS256 (Bogdanove et al. 2011). As used herein, the TAL effector domain in accordance with the present invention comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL effector molecule of the present invention is designed to target a given DNA sequence based on the above code. The number of TAL effector domains (e.g., repeats (monomers or modules)) and their specific sequence are selected based on the desired DNA target sequence. For example, TAL effector domains, e.g., repeats, may be removed or added in order to suit a specific target sequence. In an embodiment, the TAL effector molecule of the present invention comprises between 6.5 and 33.5 TAL effector domains, e.g., repeats. In an embodiment, TAL effector molecule of the present invention comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the expression repression system, e.g., the expression repressor comprising the TAL effector molecule. In general, TALE binding is inversely correlated with the number of mismatches. In some embodiments, the TAL effector molecule of a expression repressor of the present invention comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the expression repression system, e.g., the expression repressor comprising the TAL effector molecule. The binding affinity is thought to depend on the sum of matching repeat-DNA combinations. For example, TAL effector molecules having 25 TAL effector domains or more may be able to tolerate up to 7 mismatches.

In addition to the TAL effector domains, the TAL effector molecule of the present invention may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule of an expression repressor of the present invention. Accordingly, in an embodiment, a TAL effector molecule of the present invention comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

Zn Finger Molecules

In some embodiments, a targeting moiety is or comprises a Zn finger molecule. A Zn finger molecule comprises a Zn finger protein, e.g., a naturally occurring Zn finger protein or engineered Zn finger protein, or fragment thereof.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally-occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger molecules described herein may include any combination of suitable linkers between the individual zinc finger proteins and/or multi-fingered Zn finger proteins of the Zn finger molecule.

In certain embodiments, the DNA-targeting moiety comprises a Zn finger molecule comprising an engineered zinc finger protein that binds (in a sequence-specific manner) to a target DNA sequence. In some embodiments, the Zn finger molecule comprises one Zn finger protein or fragment thereof. In other embodiments, the Zn finger molecule comprises a plurality of Zn finger proteins (or fragments thereof), e.g., 2, 3, 4, 5, 6 or more Zn finger proteins (and optionally no more than 12, 11, 10, 9, 8, 7, 6, 5, 4, 3, or 2 Zn finger proteins). In some embodiments, the Zn finger molecule comprises at least three Zn finger proteins. In some embodiments, the Zn finger molecule comprises four, five or six fingers. In some embodiments, the Zn finger molecule comprises 8, 9, 10, 11 or 12 fingers. In some embodiments, a Zn finger molecule comprising three Zn finger proteins recognizes a target DNA sequence comprising 9 or 10 nucleotides. In some embodiments, a Zn finger molecule comprising four Zn finger proteins recognizes a target DNA sequence comprising 12 to 14 nucleotides. In some embodiments, a Zn finger molecule comprising six Zn finger proteins recognizes a target DNA sequence comprising 18 to 21 nucleotides.

In some embodiments, a Zn finger molecule comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18(18):5073-5084). Each cluster of zinc fingers in these proteins is able to bind to a unique target sequence and the spacing between the two target sequences can comprise many nucleotides.

In some embodiments, a targeting moiety is or comprises a DNA-binding domain from a nuclease. For example, the recognition sequences of homing endonucleases and meganucleases such as I-SceI, I-CeuI, PI-Pspl, PI-Sce, I-SceIV, I-Csml, I-PanI, I-SceII, I-Ppol, I-SceIII, I-CreI, I-TevI, I-TevII and I-TevIII are known. See also U.S. Pat. Nos. 5,420,032; 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996) J. Mol. Biol. 263:163-180; Argast, et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Patent Publication No. 2007/0117128.

### Effector Moiety

A modulating agent, e.g., disrupting agent, as described herein modulates (e.g., disrupts) the structure and/or function of a targeted genomic complex (e.g., ASMC). In some embodiments the modulating agent comprises a targeting moiety, which, by binding a targeted component of the genomic complex (e.g., ASMC), achieves the modulation. In some embodiments, a modulating agent, e.g., disrupting agent, comprises a targeting moiety and an effector moiety, wherein the effector moiety contributes to or enhances the effect of the modulating agent. In some embodiments, the effector moiety adds to the effect that binding of the targeting moiety has, e.g., on the level or occupancy of a genomic complex (e.g., ASMC) or the expression of a target gene. In some embodiments, the effector moiety has functionality unrelated to the effect that binding of the targeting moiety has. For example, effector moieties may target, e.g., bind, a genomic sequence element (e.g., a genomic sequence element in or proximal to a genomic complex (e.g., ASMC) targeted by the targeting moiety).

In some embodiments, an effector moiety modulates a biological activity, e.g., increasing or decreasing an enzymatic activity, gene expression, cell signaling, and cellular or organ function. In some embodiments, an effector moiety binds a regulatory protein, e.g., which affects transcription or translation, thereby modulating the activity of the regulatory protein. In some embodiments, an effector moiety is an activator or inhibitor (or “negative effector”) as described herein. An effector moiety may also modulate protein stability/degradation and/or transcript stability/degradation. For example, an effector moiety may target a protein for ubiqutinylation or modulate (e.g., increase or decrease ubiquitinylation) the degradation of a target protein. In some embodiments, an effector moiety inhibits an enzymatic activity by blocking an enzyme's active site. For example, an effector moiety may be or comprise methotrexate, a structural analog of tetrahydrofolate, a coenzyme for dihydrofolate reductase that binds to dihydrofolate reductase 1000-fold more tightly than its natural substrate and inhibits nucleotide base synthesis.

In some embodiments, a modulating agent, e.g., disrupting agent, comprises a targeting moiety that binds a nucleic acid, e.g., a genomic sequence element or non-genomic nucleic acid component (e.g., an ncRNA), within a genomic complex (e.g., ASMC), and is operably linked to an effector moiety that modulates the genomic complex (e.g., ASMC).

In some embodiments, an effector moiety is a chemical, e.g., a chemical that modulates a cytosine (C) or an adenine (A) (e.g., Na bisulfite, ammonium bisulfite). In some embodiments, an effector moiety has enzymatic activity (e.g., methyl transferase, demethylase, nuclease (e.g., Cas9), or deaminase activity).

An effector moiety may be or comprise one or more of a small molecule, a peptide, a nucleic acid, a nanoparticle, an aptamer, or a pharmacoagent with poor PK/PD.

In some embodiments, a modulating agent, e.g., disrupting agent, comprises one effector moiety. In some embodiments, a modulating agent, e.g., disrupting agent, comprises more than one effector moiety. In some embodiments, a modulating agent, e.g., disrupting agent, comprises 1, 2, 3, 4, 5, 6, 7, 8, 9, 10, 11, 12, 13, 14, 15, 16, 17, 18, 19, 20, or more effector domains (and optionally, less than 20, 19, 18, 17, 16, 15, 14, 13, 12, 11, 10, 9, 8, 7, 6, 5, 4, 3, or 2 effector domains). For example, a modulating agent, e.g., disrupting agent, may comprise a plurality of enzymes with a role in DNA methylation (e.g., one or more methyltransferases, demethylases, or DNA topology modifying enzymes). In some embodiments, a modulating agent, e.g., disrupting agent, comprises a linker, e.g., an amino acid linker, connecting the targeting moiety and the effector moiety. In some embodiments, a linker comprises 2 or more amino acids, e.g., one or more GS sequences. In some embodiments wherein a modulating agent, e.g., disrupting agent, comprises a plurality of effector moieties, the modulating agent comprises linkers between each of the moieties.

In some embodiments, a modulating agent, e.g., disrupting agent, e.g., effector moiety, may comprise a peptide ligand, a full-length protein, a protein fragment, an antibody, an antibody fragment, and/or a targeting aptamer. In some embodiments, the protein of a modulating agent, e.g., disrupting agent, may bind a receptor such as an extracellular receptor, neuropeptide, hormone peptide, peptide drug, toxic peptide, viral or microbial peptide, synthetic peptide, or agonist or antagonist peptide.

In some embodiments, a modulating agent, e.g., disrupting agent, e.g., effector moiety, may comprise antigens, antibodies, antibody fragments such as, e.g. single domain antibodies, ligands, and receptors such as, e.g., glucagon-like peptide-1 (GLP-1), GLP-2 receptor 2, cholecystokinin B (CCKB), and somatostatin receptor, peptide therapeutics such as, e.g., those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptide or protein moieties for use in effector moieties as described herein may also include small antigen-binding peptides, e.g., antigen binding antibody or antibody-like fragments, such as, e.g., single chain antibodies, nanobodies (see, e.g., Steeland et al. 2016. Nanobodies as therapeutics: big opportunities for small antibodies. Drug Discov Today: 21(7):1076-113). Such small antigen binding peptides may bind, e.g. a cytosolic antigen, a nuclear antigen, an intra-organellar antigen.

In some embodiments, a modulating agent, e.g., disrupting agent, e.g., an effector moiety, comprises a dominant negative component (e.g., dominant negative moiety), e.g., a protein that recognizes and binds a sequence (e.g., an anchor sequence, e.g., a CTCF binding motif), but with an inactive (e.g., mutated) dimerization domain, e.g., a dimerization domain that is unable to form a functional anchor sequence-mediated conjunction), or binds to a component of a genomic complex (e.g., a transcription factor subunit, etc.) preventing formation of a functional transcription factor, etc. For example, the Zinc Finger domain of CTCF can be altered so that it binds a specific anchor sequence (by adding zinc fingers that recognize flanking nucleic acids), while the homo-dimerization domain is altered to prevent the interaction between engineered CTCF and endogenous forms of CTCF. In some embodiments, a dominant negative component comprises a synthetic nucleating polypeptide with a selected binding affinity for an anchor sequence within a target anchor sequence-mediated conjunction. In some embodiments, binding affinity may be at least 10%, 15%, 20%, 25%, 30%, 35%, 40%, 45%, 50%, 55%, 60%, 65%, 70%, 75%, 80%, 85%, 90%, 95%, or higher or lower than binding affinity of an endogenous nucleating polypeptide (e.g., CTCF) that associates with a target anchor sequence. A synthetic nucleating polypeptide may have between 30-90%, 30-85%, 30-80%, 30-70%, 50-80%, 50-90% amino acid sequence identity to a corresponding endogenous nucleating polypeptide. A nucleating polypeptide may modulate (e.g., disrupt), such as through competitive binding, e.g., competing with binding of an endogenous nucleating polypeptide to its anchor sequence.

In some aspects, a modulating agent, e.g., disrupting agent, e.g., effector moiety, comprises an antibody or fragment thereof (e.g., the targeting or effector moiety comprises an antibody). In some embodiments, gene expression is altered via use of effector moieties that are or comprise one or more antibodies or fragments thereof. In some embodiments, gene expression is altered via use of effector moieties that are or comprise one or more antibodies (or fragments thereof) and dCas9. In some embodiments, an antibody or fragment thereof is targeted to a particular genomic complex (e.g., ASMC). In some embodiments, more than one antibody or fragment thereof (e.g., more than one of identical antibodies or one or more distinct antibodies (e.g., at least two antibodies, where each antibody is a different antibody)) is targeted to a particular genomic complex (e.g., ASMC).

In some embodiments, gene expression is altered, e.g., decreased, via use of a modulating agent, e.g., disrupting agent, e.g., effector moiety, that comprises one or more antibodies or fragments thereof and dCas9. In some embodiments, one or more antibodies or fragments thereof is/are targeted to a particular genomic complex (e.g., ASMC) via dCas9 and target-specific guide RNA.

In some embodiments, an antibody or fragment thereof for use in a modulating agent, e.g., disrupting agent, may be monoclonal or polyclonal. An antibody may be a fusion, a chimeric antibody, a non-humanized antibody, a partially or fully humanized antibody, etc. As will be understood by one of skill in the art, format of antibody(ies) used for targeting may be the same or different depending on a given target.

In some embodiments, a modulating agent, e.g., disrupting agent, e.g., effector moiety, comprises a conjunction nucleating molecule, a nucleic acid encoding a conjunction nucleating molecule, or a combination thereof. In some embodiments, an effector moiety comprises a conjunction nucleating molecule, a nucleic acid encoding a conjunction nucleating molecule, or a combination thereof. A conjunction nucleating molecule may be, e.g., CTCF, cohesin, USF1, YY1, TATA-box binding protein associated factor 3 (TAF3), ZNF143 binding motif, or another polypeptide that promotes formation of an anchor sequence-mediated conjunction. A conjunction nucleating molecule may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. A conjunction nucleating molecule may modulate DNA interactions within or around the anchor sequence-mediated conjunction. For example, a conjunction nucleating molecule can recruit other factors to an anchor sequence that alters an anchor sequence-mediated conjunction formation or disruption.

A conjunction nucleating molecule may also have a dimerization domain for homo- or heterodimerization. One or more conjunction nucleating molecules, e.g., endogenous and engineered, may interact to form an anchor sequence-mediated conjunction. In some embodiments, a conjunction nucleating molecule is engineered to further include a stabilization domain, e.g., cohesion interaction domain, to stabilize an anchor sequence-mediated conjunction. In some embodiments, a conjunction nucleating molecule is engineered to bind a target sequence, e.g., target sequence binding affinity is modulated. In some embodiments, a conjunction nucleating molecule is selected or engineered with a selected binding affinity for an anchor sequence within an anchor sequence-mediated conjunction.

Conjunction nucleating molecules and their corresponding anchor sequences may be identified through use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChIA-PET analysis using a bait, such as Cohesin, YY1 or USF1, ZNF143 binding motif, and MS to identify complexes that are associated with a bait.

In some embodiments, a modulating agent, e.g., disrupting agent, e.g., effector moiety, comprises a DNA-binding domain of a protein. In some such embodiments, the targeting moiety of the modulating agent may be or comprise the DNA-binding domain. In some embodiments, one or more of a targeting moiety and/or an effector moiety is or comprises a DNA-binding domain.

In some embodiments, a DNA binding domain of an effector moiety enhances or alters targeting of a modulating agent, e.g., disrupting agent, but does not alone achieve complete targeting by a modulating agent (e.g., the targeting moiety is still needed to achieve targeting of the modulating agent). In some embodiments, a DNA binding domain enhances targeting of a modulating agent, e.g., disrupting agent. In some embodiments, a DNA binding domain enhances efficacy of a modulating agent, e.g., disrupting agent. DNA-binding proteins have distinct structural motifs, e.g., that play a key role in binding DNA, known to those of skill in the art. In some embodiments, a DNA-binding domain comprises a helix-turn-helix (HTH) motif, a common DNA recognition motif in repressor proteins. Such a motif comprises two helices, one of which recognizes DNA (aka recognition helix) with side chains providing binding specificity. Such motifs are commonly used to regulate proteins that are involved in developmental processes. Sometimes more than one protein competes for the same sequence or recognizes the same DNA fragment. Different proteins may differ in their affinity for the same sequence, or DNA conformation, respectively through H-bonds, salt bridges and Van der Waals interactions.

In some embodiments, a DNA-binding domain comprises a helix-hairpin-helix (HhH) motif. DNA-binding proteins with a HhH structural motif may be involved in non-sequence-specific DNA binding that occurs via the formation of hydrogen bonds between protein backbone nitrogens and DNA phosphate groups.

In some embodiments, a DNA-binding domain comprises a helix-loop-helix (HLH) motif. DNA-binding proteins with an HLH structural motif are transcriptional regulatory proteins and are principally related to a wide array of developmental processes. An HLH structural motif is longer, in terms of residues, than HTH or HhH motifs. Many of these proteins interact to form homo- and hetero-dimers. A structural motif is composed of two long helix regions, with an N-terminal helix binding to DNA, while a complex region allows the protein to dimerize.

In some embodiments, a DNA-binding domain comprises a leucine zipper motif. In some transcription factors, a dimer binding site with DNA forms a leucine zipper. This motif includes two amphipathic helices, one from each subunit, interacting with each other resulting in a left handed coiled-coil super secondary structure. A leucine zipper is an interdigitation of regularly spaced leucine residues in one helix with leucines from an adjacent helix. Mostly, helices involved in leucine zippers exhibit a heptad sequence (abcdefg) with residues a and d being hydrophobic and other residues being hydrophilic. Leucine zipper motifs can mediate either homo- or heterodimer formation.

In some embodiments, a DNA-binding domain comprises a Zn finger domain, where a Zn⁺⁺ ion is coordinated by 2 Cys and 2 His residues. Such a transcription factor includes a trimer with the stoichiometry ββ′α. An apparent effect of Zn⁺⁺ coordination is stabilization of a small complex structure instead of hydrophobic core residues. Each Zn-finger interacts in a conformationally identical manner with successive triple base pair segments in the major groove of the double helix. Protein-DNA interaction is determined by two factors: (i) H-bonding interaction between α-helix and DNA segment, mostly between Arg residues and Guanine bases. (ii) H-bonding interaction with DNA phosphate backbone, mostly with Arg and His. An alternative Zn-finger motif chelates Zn⁺⁺ with 6 Cys.

In some embodiments, a DNA-binding domain comprises a TATA box binding protein (TBP). TBP was first identified as a component of the class II initiation factor TFIID. These binding proteins participate in transcription by all three nuclear RNA polymerases acting as subunit in each of them. Structure of TBP shows two α/β structural domains of 89-90 amino acids. The C-terminal or core region of TBP binds with high affinity to a TATA consensus sequence (TATAa/tAa/t, SEQ ID NO: 3) recognizing minor groove determinants and promoting DNA bending. TBP resemble a molecular saddle. The binding side is lined with central 8 strands of a 10-stranded anti-parallel β-sheet. The upper surface contains four α-helices and binds to various components of transcription machinery.

In some embodiments, a DNA-binding domain is or comprises a transcription factor. Transcription factors (TFs) may be modular proteins containing a DNA-binding domain that is responsible for specific recognition of base sequences and one or more effector domains that can activate or repress transcription. TFs interact with chromatin and recruit protein complexes that serve as coactivators or corepressors.

In some embodiments, a modulating agent, e.g., a disrupting agent, e.g., an effector moiety, comprises one or more RNAs (e.g. gRNA) and dCas9. In some embodiments, one or more RNAs is/are targeted to a particular genomic complex (e.g., ASMC) via dCas9 and target-specific guide RNA. As will be understood by one of skill in the art, RNAs used for targeting may be the same or different depending on a given target.

In some embodiments, gene expression is altered via use of a modulating agent, e.g., disrupting agent, comprising an effector moiety, that comprises an antibody or fragment thereof and dCas9. In some embodiments, one or more RNAs is/are targeted to a particular genomic complex via dCas9 and target-specific guide RNA. In some embodiments, a modulating agent, e.g., disrupting agent, e.g., an effector moiety, comprises a nucleic acid sequence, e.g., a guide RNA (gRNA). In some embodiments, a gRNA is complementary to a nucleic acid participating in a genomic complex (e.g., ASMC), e.g., a genomic sequence element (e.g., anchor sequence) or a ncRNA (e.g., eRNA).

In some embodiments, an epigenetic modifying moiety comprises a gRNA, antisense DNA, or triplex forming oligonucleotide used as a DNA target and steric presence in the vicinity of the genomic complex (e.g., ASMC), e.g., in the vicinity of the anchoring sequence. A gRNA recognizes specific DNA sequences (e.g., an anchor sequence, a CTCF anchor sequence, flanked by sequences that confer sequence specificity). A gRNA may include additional sequences that interfere with conjunction nucleating molecule sequence to act as a steric blocker. In some embodiments, a gRNA is combined with one or more peptides, e.g., S-adenosyl methionine (SAM), that acts as a steric presence to interfere with a conjunction nucleating molecule.

In some embodiments, a modulating agent, e.g., disrupting agent, e.g., effector moiety, comprises an RNAi molecule. Certain RNA agents can inhibit gene expression through a biological process using RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207). In some embodiments, the present disclosure provides compositions to inhibit expression of a gene encoding a polypeptide described herein, e.g., a conjunction nucleating molecule or epigenetic modifying agent.

RNAi molecules comprise a sequence substantially complementary, or fully complementary, to all or a fragment of a target gene. RNAi molecules may complement sequences at a boundary between introns and exons to prevent maturation of newly-generated nuclear RNA transcripts of specific genes into mRNA for transcription. RNAi molecules complementary to specific genes can hybridize with an mRNA for that gene and prevent its translation. An antisense molecule can be, for example, DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG). An antisense molecule may be comprised of synthetic nucleotides.

RNAi molecules can be provided to the cell as “ready-to-use” RNA synthesized in vitro or as an antisense gene transfected into cells which will yield RNAi molecules upon transcription. Hybridization with mRNA results in degradation of a hybridized molecule by RNAse H and/or inhibition of formation of translation complexes. Both result in a failure to produce a product of an original gene.

Length of an RNAi molecule that hybridizes to a transcript of interest should be around 10 nucleotides, between about 15 or 30 nucleotides, or about 15, 16, 17, 18, 19, 20, 21, 22, 23, 24, 25, 26, 27, 28, 29, 30 or more nucleotides. Degree of identity of an antisense sequence to a targeted transcript should be at least 75%, at least 80%, at least 85%, at least 90%, or at least 95%.

RNAi molecules may also comprise overhangs, i.e. typically unpaired, overhanging nucleotides which are not directly involved in a double helical structure normally formed by a core sequences of herein defined pair of sense strand and antisense strand. RNAi molecules may contain 3′ and/or 5′ overhangs of about 1-5 bases independently on each of a sense and antisense strand. In some embodiments, both sense and antisense strands contain 3′ and 5′ overhangs. In some embodiments, one or more 3′ overhang nucleotides of one strand base (e.g. sense) pairs with one or more 5′ overhang nucleotides of the other strand (e.g. antisense). In some embodiments, one or more 3′ overhang nucleotides of one strand base (e.g. sense) do not pair with the one or more 5′ overhang nucleotides of the other strand (e.g. antisense). Sense and antisense strands of an RNAi molecule may or may not contain the same number of nucleotide bases. Antisense and sense strands may form a duplex wherein a 5′ end only has a blunt end, a 3′ end only has a blunt end, both a 5′ and 3′ ends are blunt ended, or neither a 5′ end nor the 3′ end are blunt ended. In some embodiments, one or more nucleotides in an overhang contains a thiophosphate, phosphorothioate, deoxynucleotide inverted (3′ to 3′ linked) nucleotide or is a modified ribonucleotide or deoxynucleotide.

In some embodiments, a modulating agent, e.g., disrupting agent, e.g., effector moiety, comprises an siRNA molecule, shRNA molecule, or miRNA molecule. Small interfering RNA (siRNA) molecules comprise a nucleotide sequence that is identical to about 15 to about 25 contiguous nucleotides of a target mRNA. In some embodiments, an siRNA sequence commences with a dinucleotide AA, comprises a GC-content of about 30-70% (about 30-60%, about 40-60%, or about 45%-55%), and does not have a high percentage identity to any nucleotide sequence other than a target in a genome of a mammal in which it is to be introduced, for example as determined by standard BLAST search.

siRNAs and shRNAs resemble intermediates in processing pathway(s) of endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave an mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from an miRNA's 5′ end (Rajewsky, Nat Genet 38 Suppl:S8-13, 2006; Lim et al., Nature 433:769-773, 2005). This region is known as a seed region. Because siRNAs and miRNAs are interchangeable, exogenous siRNAs downregulate mRNAs with seed complementarity to an siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences for use in miRNA molecules can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in relevant literature. RNAi molecules are readily designed and produced by technologies known in the art. In addition, there are computational tools that increase chances of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

The RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, an RNAi molecule can contain a sequence that has complementarity to sequences that are shared amongst different gene targets or are unique for a specific gene target. In some embodiments, an RNAi molecule can be designed to target conserved regions of an RNA sequence having homology between several genes thereby targeting several genes in a gene family (e.g., different gene isoforms, splice variants, mutant genes, etc.). In some embodiments, an RNAi molecule can be designed to target a sequence that is unique to a specific RNA sequence of a single gene.

In some embodiments, an RNAi molecule targets a sequence encoding a component of a genomic complex or transcription complex, e.g., a conjunction nucleating molecule, e.g., CTCF, cohesin, USF1, YY1, TATA-box binding protein associated factor 3 (TAF3), ZNF143, or another polypeptide that promotes the formation of an anchor sequence-mediated conjunction, or an epigenetic modifying agent, e.g., an enzyme involved in post-translational modifications including, but are not limited to, DNA methylases (e.g., DNMT3a, DNMT3b, DNMTL), DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), and others. In some embodiments, the RNAi molecule targets a protein deacetylase, e.g., sirtuin 1, 2, 3, 4, 5, 6, or 7. In some embodiments, the present disclosure provides a composition comprising an RNAi that targets a conjunction nucleating molecule, e.g., CTCF.

In some embodiments, an RNAi molecule targets a nucleic acid sequence that is part of a genomic complex (e.g. ncRNA, e.g., eRNA). In some embodiments, a modulating agent, e.g., fusion molecule, e.g., the targeting moiety or effector moiety of a fusion molecule, comprises an RNAi molecule that targets an eRNA that is part of a genomic complex (e.g., ASMC).

A modulating agent, e.g., disrupting agent, e.g., effector moiety, may comprise an aptamer, such as an oligonucleotide aptamer or a peptide aptamer. Aptamer moieties are oligonucleotide or peptide aptamers.

A modulating agent, e.g., disrupting agent, e.g., effector moiety, may comprise an oligonucleotide aptamer. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers possess desirable storage properties, and elicit little or no immunogenicity in therapeutic applications.

Both DNA and RNA aptamers show robust binding affinities for various targets. For example, DNA and RNA aptamers have been selected for t lysozyme, thrombin, human immunodeficiency virus trans-acting responsive element (HIV TAR), https://en.wikipedia.org/wiki/Aptamer-cite_note-10 hemin, interferon γ, vascular endothelial growth factor (VEGF), prostate specific antigen (PSA), dopamine, and the non-classical oncogene, heat shock factor 1 (HSF1).

Diagnostic techniques for aptamer based plasma protein profiling includes aptamer plasma proteomics. This technology will enable future multi-biomarker protein measurements that can aid diagnostic distinction of disease versus healthy states.

A modulating agent, e.g., disrupting agent, e.g., effector moiety, may comprise a peptide aptamer moiety. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide complexes of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer complex attached to a transcription factor binding domain is screened against a target protein attached to a transcription factor activating domain. In vivo binding of a peptide aptamer to its target via this selection strategy is detected as expression of a downstream yeast marker gene. Such experiments identify particular proteins bound by aptamers, and protein interactions that aptamers disrupt, to cause a given phenotype. In addition, peptide aptamers derivatized with appropriate functional moieties can cause specific post-translational modification of their target proteins, or change subcellular localization of the targets.

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently a yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. Peptides panned from combinatorial peptide libraries have been stored in a special database with named MimoDB.

### Effector Moieties that Negatively Effect Genomic Complexes

In some embodiments, an effector moiety reduces the level of a genomic complex, e.g., an anchor sequence-mediated conjunction, (e.g., when a cell has been contacted with a modulating agent (e.g., disrupting agent) comprising the effector moiety, or when the effector moiety has been co-localized to the genomic complex component by the targeting moiety) as compared with when it is absent. In some embodiments, the level of a genomic complex (e.g., ASMC) decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the effector moiety relative to the absence of said modulating agent. In some embodiments, the presence of the effector moiety alters, e.g., decreases, occupancy of the genomic complex (e.g., ASMC) at a genomic sequence element (e.g., a target gene, or an enhancer associated with a targeted eRNA). In some embodiments, occupancy decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the effector moiety relative to the absence of said modulating agent.

In some embodiments, the occupancy of a genomic complex (e.g., ASMC) at a genomic sequence element (e.g., a gene, promoter, or enhancer, e.g., associated with the genomic or transcription complex) is decreased in the presence of a modulating agent, e.g., disrupting agent, comprising the effector moiety relative to the absence of said modulating agent. In some embodiments, the presence of the effector moiety alters, e.g., decreases, occupancy of the genomic complex (e.g., ASMC) at a genomic sequence element by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the effector moiety relative to the absence of said modulating agent.

In some embodiments, the occupancy of a targeted component in/at the genomic complex (e.g., ASMC) is decreased in the presence of a modulating agent, e.g., disrupting agent, comprising the effector moiety relative to the absence of said modulating agent. In some embodiments, the presence of the effector moiety alters, e.g., decreases, occupancy of a targeted component in/at the genomic complex (e.g., ASMC) by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the effector moiety relative to the absence of said modulating agent.

In some embodiments, a modulating agent (e.g., disrupting agent), e.g., an effector moiety, alters (e.g., decrease) the integrity index of a targeted genomic complex (e.g., ASMC) by at least 10%, 15%, 20%, 25%, 30%, 35%, 40%, 45%, 50%, 55%, 60%, 65%, 70%, 75%, 80%, 85%, 90%, 95%, or more. In some embodiments, a modulating agent (e.g., disrupting agent), e.g., an effector moiety, decreases the integrity index of a targeted genomic complex (e.g., ASMC) by at least 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, or 0.9 (and optionally less than 1, 0.95, 0.9, 0.85, 0.8, 0.75, 0.7, 0.65, 0.6, 0.55, or 0.5).

In some embodiments, a modulating agent, e.g., disrupting agent, that disrupts an interaction between a genomic sequence element and another genomic complex component or transcription factor comprises a effector moiety that decreases the dimerization of an endogenous nucleating polypeptide when present as compared with when the effector moiety is absent.

In some embodiments, an effector moiety alters, e.g., decreases, the level of a genomic complex (e.g., ASMC) comprising a targeted component.

In some embodiments, an effector moiety alters, e.g., decreases, the expression of a target gene associated with the genomic complex (e.g., ASMC) comprising a targeted component. In some embodiments, the expression of the target gene decreases by at least 10, 20, 30, 40, 50, 60, 70, 80, 90, or 100% (and optionally, up to 100, 90, 80, 70, 60, 50, 40, 30, or 20%) in the presence of a modulating agent, e.g., disrupting agent, comprising the effector moiety relative to the absence of said modulating agent.

In some embodiments, a modulating agent, e.g., disrupting agent, comprises a targeting moiety that targets, e.g., binds, a nucleic acid component of a genomic complex (e.g., ASMC), and an effector moiety that provides a steric presence (e.g., to inhibit binding of another genomic complex component). An effector moiety may comprise a dominant negative moiety or fragment thereof (e.g., a protein that recognizes and binds a genomic complex component (e.g., a genomic sequence element, e.g., an anchor sequence, (e.g., a CTCF binding motif)) but with an alteration (e.g., mutation) preventing formation of a functional genomic complex (e.g., ASMC)), a polypeptide that interferes with transcription factor binding or function (e.g., contact between a transcription factor and its target sequence to be transcribed), a nucleic acid sequence ligated to a small molecule that imparts steric interference, or any other combination of a recognition element and a steric blocker.

An exemplary effector moiety may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as KRAB domain.

Genetic Modifying Moieties

In some embodiments, a modulating (e.g., disrupting) agent comprises an effector moiety that is or comprises a genetic modifying moiety (e.g., components of a gene editing system). In some embodiments, a genetic modifying moiety comprises one or more components of a gene editing system. Genetic modifying moieties may be used in a variety of contexts including but not limited to gene editing. For example, such moieties may be used to localize an effector moiety to a genetic locus, e.g., so that the modulating agent, e.g., effector moiety, may physically modify, genetically modify, and/or epigenetically modify a target sequences, e.g., anchor sequence.

In some embodiments, a genetic modifying moiety may target one or more nucleotides, such as through a gene editing system, of a sequence. In some embodiments, a genetic modifying moiety binds a genomic sequence element and alters a genomic complex (e.g., ASMC), e.g., alters topology of an anchor sequence-mediated conjunction.

In some embodiments, a genetic modifying moiety targets one or more nucleotides of genomic DNA, e.g., such as through CRISPR, TALEN, dCas9, oligonucleotide pairing, recombination, transposon, within or as a component of a genomic complex (e.g. within an anchor sequence-mediated conjunction) for substitution, addition or deletion.

In some embodiments, a genetic modifying moiety introduces a targeted alteration into one or more nucleotides of genomic DNA within a genomic complex (e.g., ASMC), wherein the alteration modulates transcription of a gene, e.g., in a human cell. In some embodiments, a genetic modifying moiety introduces a targeted alteration into an ncRNA or eRNA that is part of a genomic complex (e.g., an anchor sequence-mediated conjunction), wherein the alteration modulates transcription of a gene associated with the genomic complex. A targeted alteration may include a substitution, addition, or deletion of one or more nucleotides, e.g., of an anchor sequence within an anchor sequence-mediated conjunction. A genetic modifying moiety may bind an anchor sequence of an anchor sequence-mediated conjunction and a targeting moiety introduce a targeted alteration into an anchor sequence to modulate transcription, in a human cell, of a gene in an anchor sequence-mediated conjunction. In some embodiments, a targeted alteration alters at least one of a binding site for a nucleating polypeptide, e.g., altering binding affinity for an anchor sequence within an anchor sequence-mediated conjunction, an alternative splicing site, and a binding site for a non-translated RNA.

In some embodiments, a genetic modifying moiety edits a component of a genomic complex (e.g., a sequence in an anchor sequence-mediated conjunction) via at least one of the following: providing at least one exogenous anchor sequence; an alteration in at least one nucleating polypeptide binding motif, such as by altering binding affinity for a nucleating polypeptide; a change in an orientation of at least one nucleating polypeptide binding motif, such as a CTCF binding motif; and a substitution, addition or deletion in at least one anchor sequence, such as a CTCF binding motif.

Exemplary gene editing systems whose components may be suitable for use in genetic modifying moieties include clustered regulatory interspaced short palindromic repeat (CRISPR) system (e.g., a CRISPR/Cas molecule), zinc finger nucleases (ZFNs) (e.g., a Zn Finger molecule), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 Jul. 30, 46:1-8; and Zheng et al., Precise gene deletion and replacement using the CRISPR/Cas9 system in human cells. BioTechniques, Vol. 57, No. 3, September 2014, pp. 115-124.

For example, in some embodiments, a genetic modifying moiety is site-specific and comprises a Cas nuclease (e.g., Cas9) and a site-specific guide RNA, as described further herein. In some embodiments, a genetic modifying moiety comprises a Cas nuclease (e.g., Cas9) and a site-specific guide RNA. In some embodiments, a Cas nuclease is enzymatically inactive, e.g., a dCas9, as described further herein.

In some embodiments, a genetic modifying moiety may comprise a polypeptide (e.g. peptide or protein moiety) linked to a gRNA and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to a targeted sequence. Fusions of a catalytically inactive endonuclease, e.g., a dead Cas9 (dCas9, e.g., D10A; H840A) tethered with all or a portion of (e.g., biologically active portion of) an (one or more) effector domain (e.g., epigenome editors including but not restricted to: DNMT3a, DNMT3L, DNMT3b, KRAB domain, Tetl, p300, VP64 and fusions of the aforementioned) create himeric proteins that can be linked to a polypeptide to guide a provided composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

As used herein, a “biologically active portion of an effector domain” is a portion that maintains function (e.g. completely, partially, minimally) of an effector domain (e.g., a “minimal” or “core” domain). In some embodiments, fusion of a dCas9 with all or a portion of one or more effector domains of an epigenetic modifying agent (such as a DNA methylase or enzyme with a role in DNA demethylation, e.g., DNMT3a, DNMT3b, DNMT3L, a DNMT inhibitor, combinations thereof, TET family enzymes, protein acetyl transferase or deacetylase, dCas9-DNMT3a/3L, dCas9-DNMT3a/3L/KRAB, dCas9/VP64) creates a chimeric protein that is linked to the polypeptide and useful in the methods described herein. An effector moiety comprising such a chimeric protein is referred to as either a genetic modifying moiety (because of its use of a gene editing system component, Cas9) or an epigenetic modifying moiety (because of its use of an effector domain of an epigenetic modifying agent).

In some embodiments, provided technologies are described as comprising a gRNA that specifically targets a target gene. In some embodiments, the target gene is an oncogene, a tumor suppressor, or a nucleotide repeat disease related gene.

In some embodiments, technologies provided herein include methods of delivering one or more genetic modifying moieties (e.g., CRISPR system components) described herein to a subject, e.g., to a nucleus of a cell or tissue of a subject, by linking such a moiety to a targeting moiety as part of a fusion molecule.

Epigenetic Modifying Moieties

In some embodiments, an effector moiety is or comprises an epigenetic modifying moiety that modulates the two-dimensional structure of chromatin (i.e., that modulate structure of chromatin in a way that would alter its two-dimensional representation).

Epigenetic modifying moieties useful in methods and compositions of the present disclosure include agents that affect, e.g., DNA methylation, histone acetylation, and RNA-associated silencing. In some embodiments, methods provided herein involve sequence-specific targeting of an epigenetic enzyme (e.g., an enzyme that generates or removes epigenetic marks, e.g., acetylation and/or methylation). Exemplary epigenetic enzymes that can be targeted to a genomic sequence element as described herein include DNA methylases (e.g., DNMT3a, DNMT3b, DNMTL), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an epigenetic modifying moiety comprises a histone methyltransferase activity (e.g., a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof). In some embodiments, an epigenetic modifying moiety comprises a histone demethylase activity (e.g., a protein chosen from KDM1A (i.e., LSD1), KDM1B (i.e., LSD2), KDM2A, KDM2B, KDMSA, KDMSB, KDMSC, KDMSD, KDM4B, NO66, or a functional variant or fragment of any thereof). In some embodiments, an epigenetic modifying moiety comprises a histone deacetylase activity (e.g., a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, an epigenetic modifying moiety comprises a DNA methyltransferase activity (e.g., a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof). In some embodiments, an epigenetic modifying moiety comprises a DNA demethylase activity (e.g., a protein chosen from TET1, TET2, TET3, or TDG, or a functional variant or fragment of any thereof). In some embodiments, an epigenetic modifying moiety comprises a transcription repressor activity (e.g., a protein chosen from KRAB, MeCP2, HP1, RBBP4, REST, FOG1, SUZ12, or a functional variant or fragment of any thereof). In some embodiments, an epigenetic modifying moiety useful herein comprises a construct described in Koferle et al. Genome Medicine 7.59 (2015):1-3 (e.g., at Table 1), incorporated herein by reference. For example, in some embodiments, an expression repressor comprises or is a construct found in Table 1 of Koferle et al., e.g., a histone acetyltransferase, histone deacetylase, histone methyltransferase, DNA demethylation, or H3K4 and/or H3K9 histone demethylase described in Table 1 (e.g., dCas9-p300, TALE-TET1, ZF-DNMT3A, or TALE-LSD1).

### Fusion Molecules

In some embodiments, a modulating agent (e.g., disrupting agent) of the present disclosure may be or comprise a fusion molecule, such as a fusion molecule that comprises two or more moieties. In some embodiments, a fusion molecule comprises one or more moieties described herein, e.g., a targeting moiety and/or effector moiety. In some embodiments, a fusion molecule comprises one or more moieties covalently connected to one another. In some embodiments, the one or more moieties of a fusion molecule are situated on a single polypeptide chain, e.g., the polypeptide portions of the one or more moieties are situated on a single polypeptide chain.

In some embodiments, for example, a fusion molecule may comprise (e.g., as part of an effector and/or targeting moiety) dCas9-DNMT (e.g., comprises dCas9 and DNMT as part of the same polypeptide chain), dCas9-DNMT-3a-3L, dCas9-DNMT-3a-3a, dCas9-DNMT-3a-3L-3a, dCas9-DNMT-3a-3L-KRAB, dCas9-KRAB, dCas9-APOBEC, APOBEC-dCas9, dCas9-APOBEC-UGI, dCas9-UGI, UGI-dCas9-APOBEC, UGI-APOBEC-dCas9, any variation of protein fusions as described herein, or other fusions of proteins or protein domains described herein.

Exemplary dCas9 fusion methods and compositions that are adaptable to methods and compositions provided by the present disclosure are known and are described, e.g., in Kearns et al., Functional annotation of native enhancers with a Cas9-histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067. Using methods known in the art, dCas9 can be fused to any of a variety of agents and/or molecules as described herein; such resulting fusion molecules can be useful in various disclosed methods.

In some embodiments, a fusion molecule may be or comprise a peptide oligonucleotide conjugate. Peptide oligonucleotide conjugates include chimeric molecules comprising a nucleic acid moiety covalently linked to a peptide moiety (such as a peptide/nucleic acid mixmer). In some embodiments, a peptide moiety may include any peptide or protein moiety described herein. In some embodiments, a nucleic acid moiety may include any nucleic acid or oligonucleotide, e.g., DNA or RNA or modified DNA or RNA, described herein.

In some embodiments, a peptide oligonucleotide conjugate comprises a peptide antisense oligonucleotide conjugate. In some embodiments, a peptide oligonucleotide conjugate is a synthetic oligonucleotide with a chemically modified backbone. A peptide oligonucleotide conjugate can bind to both DNA and RNA targets in a sequence-specific manner to form a duplex structure. When bound to double-stranded DNA (dsDNA) target, a peptide oligonucleotide conjugate replaces one DNA strand in a duplex by strand invasion to form a triplex structure and a displaced DNA strand may exist as a single-stranded D-loop.

In some embodiments, a peptide oligonucleotide conjugate may be cell- and/or tissue-specific. In some embodiments, such a conjugate may be conjugated directly to, e.g. oligos, peptides, and/or proteins, etc.

In some embodiments, a peptide oligonucleotide conjugate comprises a membrane translocating polypeptide, for example, membrane translocating polypeptides as described elsewhere herein.

Solid-phase synthesis of several peptide-oligonucleotide conjugates has been described in, for example, Williams, et al., 2010, Curr. Protoc. Nucleic Acid Chem., Chapter Unit 4.41, doi: 10.1002/0471142700.nc0441s42. Synthesis and characterization of very short peptide-oligonucleotide conjugates and stepwise solid-phase synthesis of peptide-oligonucleotide conjugates on new solid supports have been described in, for example, Bongardt, et al., Innovation Perspect. Solid Phase Synth. Comb. Libr., Collect. Pap., Int. Symp., 5th, 1999, 267-270; Antopolsky, et al., Helv. Chim. Acta, 1999, 82, 2130-2140.

In some embodiments, provided compositions are pharmaceutical compositions comprising fusion molecules as described herein.

In some aspects, the present disclosure provides cells or tissues comprising fusion molecules as described herein.

In some aspects, the present disclosure provides pharmaceutical compositions comprising fusion molecules as described herein.

### Linkers

In some embodiments, modulating agents, e.g., disrupting agents, e.g., fusion molecules, may include one or more linkers. In some embodiments, a modulating agent, e.g., fusion molecule, comprising a first moiety and a second moiety has a linker between the first and second moieties, e.g., between a targeting moiety and an effector moiety. A linker may be a chemical bond, e.g., one or more covalent bonds or non-covalent bonds. In some embodiments linkers are covalent. In some embodiments, linkers are non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first moiety from a second, e.g., a targeting moiety from an effector moiety. In some embodiments, for example, a linker can be positioned between a targeting moiety and an effector moiety, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g. polypeptide) from a modulating agent, but sufficiently stable to prevent premature cleavage.

In some embodiments, one or more moieties of a modulating agent described herein are linked with one or more linkers.

As will be known by one of skill in the art, commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of a linker in aqueous solutions by forming hydrogen bonds with water molecules, and therefore reduce unfavorable interactions between a linker and protein moieties.

Rigid linkers are useful to keep a fixed distance between domains and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of linking molecules include a hydrophobic linker, such as a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a modulating agent (e.g. two polypeptides). Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine or other hydrophobic residue. Components of a modulating agent may be linked using charge-based chemistry, such that a positively charged component of a modulating agent is linked to a negative charge of another component or nucleic acid.

In some embodiments, a modulating agent, e.g., disrupting agent, e.g., fusion molecule, has the capacity to form linkages, e.g., after administration (e.g. to a subject), to other polypeptides, to another moiety as described herein, e.g., an effector molecule, e.g., a nucleic acid, protein, peptide or other molecule, or other agents, e.g., intracellular molecules, such as through covalent bonds or non-covalent bonds. In some embodiments, one or more amino acids on a polypeptide of a modulating agent are capable of linking with a nucleic acid, such as through arginine forming a pseudo-pairing with guanosine or an internucleotide phosphate linkage or an interpolymeric linkage. In some embodiments, a nucleic acid is a DNA such as genomic DNA, RNA such as tRNA or mRNA molecule. In some embodiments, one or more amino acids on a polypeptide are capable of linking with a protein or peptide.

In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Additional Moieties

A modulating agent, e.g., disrupting agent, may further comprise one or more additional moieties (e.g., in addition to one or more targeting moieties and one or more effector moieties). In some embodiments, an additional moiety is selected from a tagging or monitoring moiety, a cleavable moiety (e.g., a cleavable moiety positioned between a DNA-targeting moiety and a repressor domain or at the N- or C-terminal end of a polypeptide), a small molecule, a membrane translocating polypeptide, or a pharmacoagent moiety.

### Compositions: Methods of Making, Formulation, Delivery, and Administration

The present disclosure, among other things, provides compositions that comprise or deliver a modulating agent, e.g., disrupting agent. In some embodiments, a modulating agent, e.g., disrupting agent, that comprises a polypeptide moiety or entity may be provided via a composition that includes the modulating agent (e.g., disrupting agent), e.g., polypeptide moiety or entity, or alternatively via a composition that includes a nucleic acid encoding the modulating agent (e.g., disrupting agent, e.g., polypeptide moiety or entity, and associated with sufficient other sequences to achieve expression of the disrupting agent, e.g., polypeptide moiety or entity, in a system of interest (e.g., in a particular cell, tissue, organism, etc).

In some embodiments, a provided composition may be a pharmaceutical composition whose active ingredient comprises or delivers a modulating agent, e.g., disrupting agent, as described herein and is provided in combination with one or more pharmaceutically acceptable excipients, optionally formulated for administration to a subject (e.g., to a cell, tissue, or other site thereof).

In some aspects, the present disclosure provides methods of delivering a therapeutic comprising administering a composition as described herein to a subject, wherein a genomic complex modulating (e.g., disrupting) agent is a therapeutic and/or wherein delivery of a therapeutic targets genomic complexes (e.g., ASMCs) characterized by an integrity index to change gene expression relative to gene expression in absence of a therapeutic.

In some aspects, a system for pharmaceutical use comprises a composition that targets a genomic complex characterized by an integrity index by disrupting a genomic complex. In some embodiments, the composition targets the genomic complex by binding an anchor sequence in the genomic complex to alter formation of an anchor sequence-mediated conjunction, wherein such a composition modulates transcription, in a human cell, of a target gene associated with the anchor sequence-mediated conjunction.

Thus, in some embodiments, the present disclosure provides compositions comprising a modulating agent (e.g., disrupting agent), or a production intermediate thereof. In some particular embodiments, the present disclosure provides compositions of nucleic acids that encode a modulating agent (e.g., disrupting agent) or polypeptide portion thereof. In some such embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc). In some embodiments, provided nucleic acids that encode a modulating agent (e.g., disrupting agent) or polypeptide portion thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc).

In some embodiments, a modulating agent (e.g., disrupting agent) is or comprises a vector, e.g., a viral vector, comprising one or more nucleic acids encoding one or more components of a modulating agent (e.g., disrupting agent) as described herein.

### Production

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter, and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter.

The present disclosure should not interpreted to be limited to use of any particular promoter or category of promoters (e.g. constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

In some embodiments, a modulating agent, e.g., disrupting agent, comprises or is a protein and may thus be produced by methods of making proteins. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise nontranscribed elements such as an origin of replication, a suitable promoter, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Proteins comprise one or more amino acids. Amino acids include any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

### Delivery

In various embodiments compositions described herein (e.g., modulating agents, e.g., disrupting agents) are pharmaceutical compositions. In some embodiments, compositions (e.g. pharmaceutical compositions) described herein may be formulated for delivery to a cell and/or to a subject via any route of administration. Modes of administration to a subject may include injection, infusion, inhalation, intranasal, intraocular, topical delivery, intercannular delivery, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein a genomic complex (e.g., ASMC) modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to epithelial, connective, muscular, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof.

In some embodiments, a composition of the present disclosure crosses a blood-brain-barrier, a placental membrane, or a blood-testis barrier.

In some embodiments, a composition as provided herein is administered systemically.

In some embodiments, administration is non-parenteral and a therapeutic is a parenteral therapeutic.

### Pharmaceutical Compositions

As used herein, the term “pharmaceutical composition” refers to an active agent (e.g., disrupting agent), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, a composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to a therapeutic alone. In some embodiments, a composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to a therapeutic alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to a therapeutic alone.

Pharmaceutical compositions described herein may be formulated for example including a carrier, such as a pharmaceutical carrier and/or a polymeric carrier, e.g., a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

Pharmaceutical uses of the present disclosure may include compositions (e.g. modulating agents, e.g., disrupting agents) as described herein. In some aspects, a system for pharmaceutical use comprises: a protein comprising a first polypeptide domain, e.g., a Cas or modified Cas protein, and a second polypeptide domain, e.g., a polypeptide having DNA methyltransferase activity or associated with demethylation or deaminase activity, in combination with at least one guide RNA (gRNA) or antisense DNA oligonucleotide that targets an ncRNA, such as an eRNA. A system is effective to alter, in at least a human cell, a genomic complex, e.g., a target anchor sequence-mediated conjunction, characterized by an integrity index.

In some embodiments, pharmaceutical compositions of the present disclosure comprise a zinc finger nuclease (ZFN), or a mRNA encoding a ZFN, that targets (e.g., cleaves) an ncRNA, such as an eRNA.

In some aspects, a system for pharmaceutical use comprises a composition that binds an ncRNA, such as an eRNA, and alters formation of a genomic complex comprising the ncRNA (e.g., eRNA), e.g., an anchor sequence-mediated conjunction, (e.g., a genomic complex characterized by an integrity index) wherein such a composition modulates transcription, in a human cell, of a target gene associated with the genomic complex, e.g., anchor sequence-mediated conjunction.

In some aspects, a system for altering, in a human cell, expression of a target gene, comprises a targeting moiety (e.g., a gRNA, a membrane translocating polypeptide) that associates with an ncRNA, such as an eRNA, associated with a target gene, and an effector moiety (e.g. an enzyme, e.g., a nuclease or deactivated nuclease (e.g., a Cas9, dCas9), a methylase, a de-methylase, a deaminase) operably linked to the targeting moiety, wherein the system is effective to alter (e.g., decrease) expression of the target gene. The targeting moiety and effector moiety may be different and separate (e.g., comprised in different physical portions of a disrupting agent) moieties. A targeting moiety and an effector moiety may be linked, e.g., covalently, e.g., by a linker. In some embodiments, a system comprises a synthetic polypeptide comprising a targeting moiety and an effector moiety. In some embodiments, a system comprises a nucleic acid vector or vectors encoding at least one of a targeting moiety and an effector moiety.

In some aspects, pharmaceutical compositions may comprise a composition that targets a genomic complex (e.g., ASMC) characterized by an integrity index by binding an anchor sequence of an anchor sequence-mediated conjunction and altering formation of an anchor sequence-mediated conjunction, wherein the composition modulates transcription, in a human cell, of a target gene associated with the genomic complex (e.g., ASMC). In some embodiments, a composition targets a genomic complex characterized by an integrity index by disrupting formation of an anchor sequence-mediated conjunction (e.g., decreases affinity of an anchor sequence to a conjunction nucleating molecule, e.g., at least 10%, 15%, 20%, 25%, 30%, 35%, 40%, 45%, 50%, 55%, 60%, 65%, 70%, 75%, 80%, 85%, 90%, 95%, or more). In some embodiments, disrupting formation comprises an alteration of integrity index by modulating affinity of an anchor sequence to a conjunction nucleating molecule, e.g., by at least 10%, 15%, 20%, 25%, 30%, 35%, 40%, 45%, 50%, 55%, 60%, 65%, 70%, 75%, 80%, 85%, 90%, 95%, or more.

In some embodiments, administration of compositions described herein improves at least one pharmacokinetic or pharmacodynamic parameter of at least one component of the composition (e.g. a pharmacoagent), such as targeting, absorption, and transport, as compared to another moiety alone, or reduces at least one toxicokinetic parameter, such as diffusion to non-target location, off-target activity, and toxic metabolism, as compared to another moiety alone (e.g., by at least 5%, 10%, 20%, 25%, 30%, 40%, 50%, 60%, 70%, 80% or more). In some embodiments, administration of compositions of the present disclosure increases a therapeutic range of at least one component of a modulating agent (e.g., by at least 5%, 10%, 20%, 25%, 30%, 40%, 50%, 60%, 70%, 80% or more). In some embodiments, administration of compositions provided herein reduces a minimum effective dose, as compared to another moiety alone (e.g., by at least 5%, 10%, 20%, 25%, 30%, 40%, 50%, 60%, 70%, 80% or more). In some embodiments, administration of compositions provided increases a maximum tolerated dose, as compared to a modulating agent alone (e.g., by at least 5%, 10%, 20%, 25%, 30%, 40%, 50%, 60%, 70%, 80% or more). In some embodiments, administration of compositions provided herein increases efficacy or decreases toxicity of a therapeutic, such as non-parenteral administration of a parenteral therapeutic. In some embodiments, administration of compositions provided herein increases a therapeutic range of a modulating agent while decreasing toxicity, as compared to a modulating agent alone (e.g., by at least 5%, 10%, 20%, 25%, 30%, 40%, 50%, 60%, 70%, 80% or more).

In some aspects, the present disclosure provides a modulating agent, e.g., a disrupting agent, comprising a targeting moiety that binds an ncRNA, such as an eRNA, and alters, e.g., decreases, formation of a genomic or transcription complex, e.g., an anchor sequence-mediated conjunction (e.g., decreases the level of the complex by at least 10%, 15%, 20%, 25%, 30%, 35%, 40%, 45%, 50%, 55%, 60%, 65%, 70%, 75%, 80%, 85%, 90%, 95%, or more).

In some aspects, a pharmaceutical composition includes a Cas protein and at least one guide RNA (gRNA) that targets a Cas protein to an ncRNA, such as an eRNA. The Cas protein should be effective to cause a mutation of the target ncRNA, such as an eRNA, that decreases formation of a genomic complex, e.g., an anchor sequence-mediated conjunction, comprising the ncRNA (e.g., eRNA), e.g., and characterized by an integrity index.

In some embodiments, a gRNA is administered in combination with a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to an ncRNA, such as an eRNA. For example, in some embodiments, one gRNA is administered, e.g., to produce an inactivating indel mutation in an ncRNA, such as an eRNA, e.g., one gRNA is administered in combination with a nuclease, e.g., wtCas9.

In some aspects, the present disclosure provides a composition comprising a nucleic acid or combination of nucleic acids that when administered to a subject in need thereof introduce a site specific alteration (e.g., insertion, deletion (e.g., knockout), translocation, inversion, single point mutation) in a target sequence of a target genomic complex (e.g., ASMC) characterized by an integrity index or of a component of a target genomic complex, e.g., an ncRNA, eRNA, thereby modulating gene expression in a subject.

### Uses

Technologies provided herein achieve modulation of structure and/or function of genomic complexes. Among other things, in some embodiments such provided technologies target genomic complexes characterized by an integrity index to modulate gene expression and, for example, enable breadth over controlling gene activity e.g., in a cell. In some embodiments, modulation of gene expression occurs via determination of integrity index scores of target genomic complexes. In some such embodiments, target genomic complexes with certain integrity index scores as described herein are targeted for modulation (e.g., disruption), wherein expression of one or more genes associated with a target genomic complex with an integrity index score falling within a provided range is altered after contact with a modulating (e.g., disrupting) agent.

In some embodiments, provided methods comprise a step of: determining specificity and/or integrity index of one or more genomic complexes (e.g., ASMCs) (e.g., integrity index of a particular ASMC) by any of the methods described herein. In some embodiments, provided methods comprise a step of: contacting a cell with a modulating agent, e.g., disrupting agent. In some embodiments, provided methods comprise a step of: delivering a modulating (e.g., disrupting) agent to a cell. In some embodiments, a step of delivering is performed ex vivo. In some embodiments, the step of delivering comprises administering a composition comprising a modulating, e.g., disrupting, agent to a subject. In some embodiments, the step of delivering comprises delivery across a cell membrane. In some embodiments, methods further comprise, prior to the step of delivering, a step of removing a cell (e.g., a mammalian cell) from a subject. In some embodiments, methods further comprise, after the step of delivering, a step of (b) administering cells (e.g., mammalian cells) to a subject. In some embodiments, a subject has a disease, disorder, or condition.

For example, in some embodiments, a cell is a mammalian somatic cell. In some embodiments, a mammalian somatic cell is a primary cell. In some embodiments, a mammalian somatic cell is a non-embryonic cell.

In some embodiments, provided methods comprise a step of: (a) administering somatic mammalian cells to a subject, wherein somatic mammalian cells were obtained from a subject, and modulating agent (e.g., disrupting agent) as described herein had been delivered ex vivo to somatic mammalian cells. In some embodiments, cells or tissue may be excised from a subject and gene expression, e.g., endogenous or exogenous gene expression, may be altered in cells or tissues characterized by a particular integrity index or range of integrity indices ex vivo prior to transplantation of cells or tissues back into a subject. Any cell or tissue may be excised and used for re-transplantation. Some examples of cells and tissues include, but are not limited to, stem cells, adipocytes, immune cells, myocytes, bone marrow derived cells, cells from the kidney capsule, fibroblasts, endothelial cells, and hepatocytes.

In some embodiments, indications that affect any one of blood, liver, immune system, neuronal system, etc. or combinations thereof may be treated by modulating gene expression through altering a genomic complex, e.g., an anchor sequence-mediated conjunction, (e.g., characterized by an integrity index) in a mammalian subject.

In some aspects, provided methods comprise altering gene expression or altering a genomic complex, e.g., an anchor sequence-mediated conjunction, characterized by an integrity index in a mammalian subject. Methods may include administering to a subject (separately or in a single pharmaceutical composition): a protein comprising a first polypeptide domain that comprises a Cas or modified Cas protein and a second polypeptide domain that comprises a polypeptide having DNA methyltransferase activity (or associated with demethylation or deaminase activity), or a nucleic acid encoding a protein comprising a first polypeptide domain that comprises a Cas or modified Cas protein and a second polypeptide domain that comprises a polypeptide having DNA methyltransferase activity (or associated with demethylation or deaminase activity), and at least one guide RNA (gRNA) that targets an ncRNA, such as an eRNA. In some embodiments, a gRNA targets a component of a genomic complex (e.g., ASMC), such as an ncRNA or eRNA.

Methods and compositions as provided herein may treat disease by targeting one or more genomic complexes (e.g., ASMCs) with a particular integrity index or range of integrity indices for disruption either stably or transiently by modulating transcription of a target nucleic acid sequence within the genomic complex. In some embodiments, the targeted genomic complex is altered to result in a stable modulation of transcription, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some other embodiments, the targeted genomic complex is altered to result in a transient modulation of transcription, such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some aspects, methods provided by the present disclosure may comprise targeting a genomic complex characterized by a particular integrity index or range of integrity indices to modify expression of a target gene, which methods may comprise administering to a cell, tissue or subject a genomic complex modulating (e.g., disrupting) agent as described herein.

In some aspects, the present disclosure provides methods of modifying expression of a target gene, comprising altering a genomic complex, e.g., an anchor sequence-mediated conjunction, characterized by an integrity index and associated with a target gene, wherein an alteration modulates transcription of a target gene. In some embodiments, the alteration is disruption, and such a disruption may be any change in physical association of genomic complex components that results in a change in integrity index score, for example, due to disruption of a target anchor sequence-mediated conjunction.

In some embodiments, provided technologies may comprise inducibly altering a genomic complex or component of a genomic complex (e.g., ncRNA, eRNA, transcription factor, transcription regulator, etc.) characterized by a particular integrity index or range of integrity indices. Use of an inducible alteration to a genomic complex or component of a genomic complex (e.g., ncRNA, transcription factor, etc.) provides a molecular switch to alter an integrity index of the genomic complex. In some embodiments, a molecular switch is capable of turning on an alteration when desired resulting in the genomic complex having a different integrity index. In some embodiments, a molecular switch is capable of turning off an alteration when it is not desired resulting in the genomic complex having a different integrity index. In some embodiments, a molecular switch is capable of both turning on and turning off an alteration, as desired. For example, in some embodiments, a molecular switch causes a particular genomic complex disrupting agent to disrupt a target genomic complex. In some embodiments, once an inducible genomic complex disrupting agent is turned “on”, the disruption of the target genomic complex is reversible. In some such embodiments, the molecular switch may be turned on to catalyze the disruption and then turned off, after which the genomic complex recovers from disruption. In some embodiments, once an inducible genomic complex disrupting agent is turned “on”, the disrupting of the target genomic complex is irreversible. In some such embodiments, even if the inducible genomic complex disrupting agent is turned “off”, the disrupted genomic complex will not recover from disrupting. Examples of systems used for inducing alterations include, but are not limited to an inducible targeting moiety based on a prokaryotic operon, e.g., the lac operon, transposon Tn10, tetracycline operon, and the like, and an inducible targeting moiety based on a eukaryotic signaling pathway, e.g., steroid receptor-based expression systems, e.g., the estrogen receptor or progesterone-based expression system, the metallothionein-based expression system, the ecdysone-based expression system, e.g. any system that methylates or demethylates DNA, etc. In some embodiments, provided methods and compositions may include an inducible nucleating polypeptide or other protein that interacts with an anchor sequence-mediated conjunction.

In some embodiments, cells or tissue may be excised from a subject and gene expression, e.g., endogenous or exogenous gene expression, may be altered ex vivo prior to transplantation of cells or tissues back into a subject. Any cell or tissue may be excised and used for re-transplantation. Some examples of cells and tissues include, but are not limited to, stem cells, adipocytes, immune cells, myocytes, bone marrow derived cells, cells from the kidney capsule, fibroblasts, endothelial cells, and hepatocytes. In some embodiments, for example, adipose tissue from a patient may be altered ex vivo to increase energy production and lipid utilization. Modified adipose cells are returned to a patient from whom they were excised and act as “furnaces,” e.g., they uptake lipids from circulation and use them for energy production.

In some aspects, the present disclosure provides technologies for delivering a composition as provided herein to a target tissue or cell (e.g., stem cells, progenitor cells, differentiated and/or mature cells, post-mitotic cells, e.g., liver, skin, brain, caudate and/or putamen nuclei, hepatocytes, fibroblasts, CD34+ cells, CD3+ cells, etc.), where a composition includes a targeting moiety, e.g., a receptor ligand, that targets a specific tissue or cell and a therapeutic moiety. Upon administration, a composition increases targeted delivery of a therapeutic as compared to a therapeutic alone. When a composition of the present disclosure is used in combination with an existing therapeutic that suffers from diffusion or off-target effects, specificity of the therapeutic is increased. For example, a composition described herein includes a modulating (e.g., disrupting) agent comprising (e.g., linked to) a particular agent and a ligand that specifically binds a receptor on a particular target cell type. Administration of such a composition increases specificity of the agent to the target cells through a ligand-receptor interaction.

The present disclosure also provides methods of delivering a composition described herein to a subject. In some embodiments, a composition is delivered across a cellular membrane, e.g., a plasma membrane, a nuclear membrane, an organellar membrane. Current polymeric delivery technologies increase endocytic rates in certain cell types, usually cells that preferentially utilize endocytosis, such as macrophages and other cell types that rely on calcium influx to trigger endocytosis. Without being bound by any particular theory, a composition described herein is believed to aid movement of a composition across membranes typically inaccessible by most agents.

In some aspects, a kit is described that includes: (a) a nucleic acid encoding a protein comprising a first polypeptide domain that comprises a Cas or modified Cas protein and a second polypeptide domain, e.g., a polypeptide having DNA methyltransferase activity or associated with demethylation or deaminase activity, and (b) at least one guide RNA (gRNA) for targeting a protein to an anchor sequence of a target anchor sequence-mediated conjunction in a target cell. In some embodiments, a nucleic acid encoding a protein and a gRNA are in the same vector, e.g., a plasmid, an AAV vector, an AAV9 vector. In some embodiments, a nucleic acid encoding a protein and a gRNA are in separate vectors.

### Modulating Gene Expression

As will be appreciated by one of skill in the art, particular genes are known to be associated with complexes and in many cases the effect of a given genomic complex (e.g., ASMC), characterized by an integrity index, on gene expression is known. Thus, in some embodiments, as described herein, complex inhibition inhibits expression of an associated gene. In some embodiments, as described herein, complex inhibition promotes expression of an associated gene.

In some embodiments, transcription of a nucleic acid sequence is modulated, e.g., transcription of a target nucleic acid sequence, as compared with a reference value, e.g., transcription of a target sequence in absence of an altered genomic complex, e.g., anchor sequence-mediated conjunction.

In some embodiments, modulation (e.g., disruption) is based on an integrity index above a certain threshold. Thus, in some embodiments, a genomic complex (e.g., ASMC) targeted in accordance with the present disclosure is one whose integrity index is above a minimum threshold. For instance, in some embodiments, a targeted genomic complex is characterized by an integrity above approximately 0.5, reflecting a “more likely than not” incidence in a relevant cell or cell population (e.g., tissue, organism, etc). In some embodiments an integrity index is above a value between about 0.5 to about below 1.0. In some embodiments, an integrity index is greater than or equal to 0.5, 0.55, 0.6, 0.65, 0.7, 0.75, 0.8, 0.85, 0.9, 0.91, 0.92, 0.93, 0.94, 0.95, 0.96, 0.97, 0.98, or 0.99 (and optionally, has an integrity index of less than or equal to 1, 0.99, 0.95, 0.9, 0.85, 0.8, 0.75, 0.7, 0.65, or 0.6). In some embodiments, an integrity index is 0.5-1, 0.5-0.9, 0.5-0.8, 0.5-0.7, 0.5-0.6, 0.6-1, 0.6-0.9, 0.6-0.8, 0.6-0.7, 0.7-1, 0.7-0.9, 0.7-0.8, 0.8-1, 0.8-0.9, or 0.9-1.

In some embodiments, the present disclosure encompasses the insight that, in certain circumstances, while it may be desirable to target a genomic complex (e.g., ASMC) characterized by an integrity index above a particular threshold, as described above, it may not be desirable to target a genomic complex whose integrity index is too high. For example, in some embodiments, certain genomic complexes (e.g., ASMCs) with integrity indices above a certain threshold may be associated with housekeeping genes (e.g. if a given complex is associated with an active gene); if presence of such a complex is associated with expression of the housekeeping gene, then disruption of the genomic complex could have an undesirable impact on the cell(s) in which such disruption occurs. Alternatively or additionally, in some embodiments, certain genomic complexes (e.g., ASMCs) with high integrity indices (e.g., above a certain threshold) may be associated with repressed genes, where expression of the genes could have undesirable consequences on the cell(s); in such embodiments, if presence of the genomic complex is associated with repression of the repressed gene(s), then disruption of the genomic complex could have undesirable impact(s) on the cell(s) in which such disruption occurs. In some embodiments, modulation (e.g., disruption) is based on an integrity index within a certain range. Thus, in some embodiments, a genomic complex (e.g., ASMC) targeted in accordance with the present disclosure is one whose integrity index is within a certain range. In some embodiments, an integrity index is greater than or equal to 0.25, 0.3, 0.35, 0.4, 0.45, 0.5, 0.55, 0.6, 0.65, or 0.7, and less than or equal to 0.75, 0.7, 0.65, 0.6, 0.55, 0.5, 0.45, 0.4, 0.35, or 0.3. In some embodiments, an integrity index is 0.25-0.75, 0.25-0.65, 0.25-0.55, 0.25-0.45, 0.25-0.35, 0.35-0.75, 0.35-0.65, 0.35-0.55, 0.35-0.45, 0.45-0.75, 0.45-0.65, 0.45-0.55, 0.55-0.75, 0.55-0.65, or 0.65-0.75.

In some embodiments, the present disclosure defines genomic complexes of interest for targeting with a modulating agent as described herein. In some embodiments, such genomic complexes are those characterized by an integrity index within a range as described herein. In some embodiments, such genomic complexes are those characterized by an integrity index that is different in a target cell as compared with one or more non-target cell(s). That is, in some embodiments, a particular genomic complex (i.e., a genomic complex that occurs at a particular genomic location) is characterized by a different integrity index in a first cell type or developmental stage as compared with at least one second cell type or developmental stage. In some embodiments, a target genomic complex has a particular integrity index score that is greater than an integrity index score in a second cell type or developmental stage and less than an integrity index score in a third cell type or developmental stage. In some such embodiments, a genomic complex represents a candidate to target for disruption.

In some embodiments, provided are technologies for modulating expression of a gene associated with a genomic complex, e.g., an anchor sequence-mediated conjunction, characterized by an integrity index, which conjunction comprises a first anchor sequence and a second anchor sequence. A gene that is associated with an anchor sequence-mediated conjunction may be at least partially within a conjunction (that is, situated sequence-wise between first and second anchor sequences), or it may be external to a conjunction in that it is not situated sequence-wise between a first and second anchor sequences, but is located on the same chromosome and in sufficient proximity to at least a first or a second anchor sequence such that its expression can be modulated by controlling the topology of the anchor sequence-mediated conjunction. Those of ordinary skill in the art will understand that distance in three-dimensional space between two elements (e.g., between the gene and the anchor sequence-mediated conjunction) may, in some embodiments, be more relevant than distance in terms of basepairs. In some embodiments, an external but associated gene is located within 2 Mb, within 1.9 Mb, within 1.8 Mb, within 1.7 Mb, within 1.6 Mb, within 1.5 Mb, within 1.4 Mb, with 1.3 Mb, within 1.3 Mb, within 1.2 Mb, within 1.1 Mb, within 1 Mb, within 900 kb, within 800 kb, within 700 kb, within 500 kb, within 400 kb, within 300 kb, within 200 kb, within 100 kb, within 50 kb, within 20 kb, within 10 kb, or within 5 kb of the first or second anchor sequence.

In some embodiments, modulating expression of a gene comprises targeting a genomic complex (e.g., ASMC) with a particular integrity index or range of integrity indices and altering accessibility of a transcriptional control sequence to a gene. A transcriptional control sequence, whether internal or external to an anchor sequence-mediated conjunction, can be an enhancing sequence or a silencing (or repressive) sequence.

For example, in some embodiments, methods are provided for targeting a genomic complex (e.g., ASMC) with a particular integrity index or range of integrity indices and modulating expression of a gene within the genomic complex (e.g., anchor sequence-mediated conjunction) comprising a step of: contacting the first and/or second anchor sequence with a modulating agent as described herein. In some embodiments, an anchor sequence-mediated conjunction comprises at least one transcriptional control sequence that is “internal” to a conjunction in that it is at least partially located sequence-wise between first and second anchor sequences. Thus, in some embodiments, both a gene whose expression is to be modulated (the “target gene”) and a transcriptional control sequence are within an anchor sequence-mediated conjunction.

In some embodiments, a gene is separated from an internal transcriptional control sequence by at least 300, at least 400, at least 500, at least 600, at least 700, at least 800, or at least 900 base pairs. In some embodiments, a gene is separated from an internal transcriptional control sequence by at least 1.0, at least 1.2, at least 1.4, at least 1.6, or at least 1.8 kb. In some embodiments, a gene is separated from an internal transcriptional control sequence by at least 2 kb, at least 3 kb, at least 4 kb, at least 5 kb, at least 6 kb, at least 7 kb, at least 8 kb, at least 9 kb, or at least 10 kb. In some embodiments, a gene is separated from an internal transcriptional control sequence by at least 20 kb, at least 30 kb, at least 40 kb, at least 50 kb, at least 60 kb, at least 70 kb, at least 80 kb, at least 90 kb, or at least 100 kb. In some embodiments, a gene is separated from an internal transcriptional control sequence by at least 150 kb, at least 200 kb, at least 250 kb, at least 300 kb, at least 350 kb, at least 400 kb, at least 450 kb, or at least 500 kb. In some embodiments, the gene is separated from an internal transcriptional control sequence by at least 600 kb, at least 700 kb, at least 800 kb, at least 900 kb, or at least 1 Mb.

In some embodiments, an anchor sequence-mediated conjunction comprises at least one transcriptional control sequence that is “external” to the conjunction in that it is not located sequence-wise between first and second anchor sequences. (See, e.g., Types 2, 3, and 4 anchor sequence-mediated conjunctions depicted in FIG. 1.) In some embodiments, a first and/or a second anchor sequence is located within 1 Mb, within 900 kb, within 800 kb, within 700 kb, within 600 kb, within 500 kb, within 450 kb, within 400 kb, within 350 kb, within 300 kb, within 250 kb, within 200 kb, within 180 kb, within 160 kb, within 140 kb, within 120 kb, within 100 kb, within 90 kb, within 80 kb, within 70 kb, within 60 kb, within 50 kb, within 40 kb, within 30 kb, within 20 kb, or within 10 kb of an external transcriptional control sequence. In some embodiments, the first and/or the second anchor sequence is located within 9 kb, within 8 kb, within 7 kb, within 6 kb, within 5 kb, within 4 kb, within 3 kb, within 2 kb, or within 1 kb of an external transcriptional control sequence.

For example, in some embodiments, methods are provided for modulating expression of a gene external to an anchor sequence-mediated conjunction comprising a step of: contacting a first and/or second anchor sequence with a modulating agent as described herein. In some embodiments, an anchor sequence-mediated conjunction comprises at least one internal transcriptional control sequence.

In some embodiments, an anchor sequence-mediated conjunction comprises at least one external transcriptional control sequence.

Thus, among other things, the present application provides technologies for modulating gene expression by modulating genomic complexes (e.g., ASMCs) characterized by integrity indices as described herein.

In some embodiments, modulation may include inducing disruption or formation of insulated neighborhoods. In some embodiments, modulating insulated neighborhoods affects transcription by interfering with formation/reducing frequency of assembly/inducing dissociation of a genomic complex (e.g., ASMC) (e.g., characterized by an integrity index), i.e. a cellular complex responsible for mediating any regulatory effect(s) that insulated neighborhoods have on gene transcription.

In some aspects, the present disclosure provides methods that disrupt one or more genomic complexes (e.g., ASMCs) characterized by an integrity index. By way of non-limiting example, in some embodiments disruption may refer to changes in structural topology of one or more genomic complexes (e.g., ASMCs) characterized by an integrity index. In some embodiments, disruption, as used herein, may refer to changes in function of one or more genomic complexes (e.g., ASMCs) without requiring impact or change to structural topology. For example, in some embodiments, methods may include disruption of structural topology of one or more genomic complexes (e.g., ASMCs). Without wishing to be bound by any theory, in some embodiments, disruption of genomic complexes (e.g., ASMCs) may alter gene expression. Gene expression alteration may be or comprise upregulation of one or more genes relative to expression levels in absence of genomic complex (e.g., ASMC) disruption. Gene expression alteration may be or comprise downregulation of one or more genes relative to expression levels in absence of genomic complex (e.g., ASMC) disruption.

In some embodiments, disruption may be or comprise deleting one or more CTCF binding sites.

In some embodiments, disruption may be or comprise methylating one or more CTCF binding sites.

In some embodiments, disruption may be or comprise inducing degradation of non-coding RNA that is part of a genomic complex (e.g., ASMC) (e.g. between two CTCF binding sites/anchor sites) characterized by an integrity index.

In some embodiments, disruption may be or comprise interfering with assembly of one or more genomic complexes (e.g., ASMCs) (e.g. a genomic complex that would otherwise form in absence of exogenous interference) characterized by one or more integrity indices by blocking resident non-coding RNA.

### Genetic Modification

In some embodiments, technologies (e.g. methods and/or compositions) provided by the present disclosure for targeting a genomic complex with a particular integrity index or range of integrity indices may include site specific editing or mutating of a genomic sequence element (e.g., that participates in a genomic complex (e.g., ASMC) and/or is part of a gene associated therewith). For example, in some embodiments, an endogenous or naturally occurring anchor sequence may be altered to inactivate or delete an anchor sequence (e.g., thereby disrupting an anchor sequence-mediated conjunction or the genomic complex comprising said conjunction), or may be altered to mutate or replace an anchor sequence (e.g., to mutate or replace an anchor sequence with an altered anchor sequence that has an altered affinity, e.g., decreased affinity or increased affinity, to a nucleating protein) to modulate strength of a targeted conjunction. In some embodiments, for example, one or a plurality of exogenous anchor sequences can be incorporated into the genome of a subject to create a non-naturally occurring anchor sequence-mediated conjunction that incorporates a target gene, e.g., in order to silence a target gene. In some embodiments, an exogenous anchor sequence can form an anchor sequence-mediated conjunction with an endogenous anchor sequence. A nucleating protein may be, e.g., CTCF, cohesin, USF1, YY1, TAF3, ZNF143 binding motif, or another polypeptide that promotes formation of an anchor sequence-mediated conjunction.

In some embodiments, technologies as provided herein may include those that alter a target sequence (e.g. a sequence that is part of or participates in a targeted genomic complex (e.g., ASMC) characterized by an integrity index).

In some embodiments, technologies as provided herein may include those that alter a target sequence (for example, an anchor sequence), which is a CTCF-binding motif: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO:1), where N is any nucleotide. A CTCF-binding motif may also be altered to be in the opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO:2).

An alteration can be introduced in a gene of a cell, e.g., in vitro, ex vivo, or in vivo.

In some cases, compositions and/or methods of the present disclosure are for altering chromatin structure, e.g., such that a two-dimensional representation of chromatin structure may change from that of a complex to a non-complex (or favor a non-complex over a complex) or vice versa, to alter a component of a genomic complex (e.g., ASMC) (e.g. a transcription factor and, e.g. its interaction with a genomic sequence), to inactivate a targeted CTCF-binding motif, e.g., an alteration abolishes CTCF binding thereby abolishing formation of a targeted conjunction, etc. In other examples, an alteration attenuates (e.g., decreases the level of) activity of a particular genomic complex component thereby decreasing or disrupting formation of a genomic complex (e.g., ASMC) characterized by an integrity index (e.g., by altering a CTCF sequence to bind with less affinity to a nucleating protein). In some embodiments, a targeted alteration increases activity of a particular genomic complex component thereby increasing or maintaining formation of a genomic complex (e.g., ASMC) characterized by an integrity index (e.g., by altering the CTCF sequence to bind with more affinity to a nucleating protein), thereby promoting formation of a targeted conjunction.

In some embodiments, provided modulating agents may comprise (i) a disrupting agent comprising an enzymatically inactive Cas polypeptide and a deaminating agent, or a nucleic acid encoding the disrupting agent; and (ii) a nucleic acid molecule (e.g. gRNA, PNA, BNA, etc), wherein the nucleic acid molecule targets a disrupting agent to a target sequence (e.g. in a genomic complex, e.g. in an anchor sequence-mediated conjunction, characterized by an integrity index) but not to at least one non-target anchor sequence (a “site-specific nucleic acid molecule”, such as described further herein).

In some embodiments, in order to introduce small mutations or a single-point mutation, a homologous recombination (HR) template can also be used. In some embodiments, an HR template is a single stranded DNA (ssDNA) oligo or a plasmid. In some embodiments, for example, for ssDNA oligo design, one may use around 100-150 bp total homology with a mutation introduced roughly in the middle, giving 50-75 bp homology arms.

In some embodiments, a nucleic acid molecule for targeting a target anchor sequence, e.g., a target sequence, is administered in combination with an HR template selected from:
(a) a nucleotide sequence comprising a target sequence of interest (e.g. target sequence that is part of or participates in a target genomic complex (e.g., ASMC));
(b) a nucleotide sequence at least 75%, 80%, 85%, 90%, 95% identical to a target sequence of interest;
(c) a nucleotide sequence comprising a target sequence of interest having at least 1, 2, 3, 4, 5, but less than 15, 12 or 10 nucleotide additions, substitutions or deletions.

### Modifying Chromatin Structure

In some embodiments, methods provided herein modulate (e.g., disrupt) chromatin structure (e.g., anchor sequence-mediated conjunctions) in order to target a genomic complex with a particular integrity index or range of integrity indices and modulate gene expression in a subject, e.g., by modifying anchor sequence-mediated conjunctions in DNA. Those skilled in the art reading the present specification will appreciate that modulations described herein may modulate chromatin structure in a way that would alter its two-dimensional representation (e.g., would add, alter, or delete a complex or a other anchor sequence-mediated conjunction); such modulations are referred to herein, in accordance with common parlance, as modulations or modification of a two-dimensional structure.

In some aspects, methods provided herein may comprise targeting a genomic complex with a particular integrity index or range of integrity indices by altering a topology of a genomic complex, e.g., an anchor sequence-mediated conjunction, to modulate transcription of a nucleic acid sequence, wherein altered topology of a genomic complex, e.g., an anchor sequence-mediated conjunction, modulates transcription of a nucleic acid sequence.

In some aspects, methods provided herein may comprise modifying a two-dimensional structure chromatin structure by altering a topology of a plurality of genomic complexes, e.g., anchor sequence-mediated conjunctions, characterized by one or more integrity indices, to modulate transcription of a nucleic acid sequence, wherein altered topology modulates transcription of a nucleic acid sequence.

In some aspects, methods provided herein may comprise modulating transcription of a nucleic acid sequence by altering a genomic complex, e.g., an anchor sequence-mediated conjunction, characterized by an integrity index, that influences transcription of a nucleic acid sequence, wherein altering a genomic complex, e.g., an anchor sequence-mediated conjunction, modulates transcription of a nucleic acid sequence.

In some embodiments, altering a genomic complex, e.g., an anchor sequence-mediated conjunction, characterized by an integrity index comprises: modifying a chromatin structure, e.g., disrupting reversibly or irreversibly a topology of a genomic complex, e.g., an anchor sequence-mediated conjunction; altering one or more nucleotides in a genomic complex, e.g., an anchor sequence-mediated conjunction, e.g., genetically modifying the sequence; epigenetically modifying a genomic complex, e.g., an anchor sequence-mediated conjunction, e.g., modulating DNA methylation at one or more sites; or forming a non-naturally occurring anchor sequence-mediated conjunction. In some embodiments, altering a genomic complex, e.g., an anchor sequence-mediated conjunction, characterized by an integrity index comprises modifying a chromatin structure.

### Epigenetic Modification

In some embodiments, provided compositions and/or methods are described herein for altering a genomic complex (e.g., ASMC) characterized by an integrity index by site specific epigenetic modification (e.g., methylation or demethylation).

In some embodiments, a modulating agent, e.g., disrupting agent, may cause epigenetic modification. For example, an endogenous or naturally occurring target sequence (e.g. a sequence within a target genomic complex (e.g., ASMC) characterized by an integrity index) may be altered to increase its methylation (e.g., decreasing interaction of a component of a genomic complex (e.g., ASMC) (e.g. a transcription factor) with a portion of a genomic sequence, decreasing binding of a nucleating protein to the anchor sequence and disrupting or preventing an anchor sequence-mediated conjunction, or may be altered to decrease its methylation (e.g., interaction of a component of a genomic complex (e.g., ASMC) (e.g. a transcription factor) with a portion of a genomic sequence, increasing binding of a nucleating protein to an anchor sequence and promoting or increasing strength of an anchor sequence-mediated conjunction, etc.).

In some particular embodiments, a modulating agent may be or comprise a disrupting agent, for example comprising a site-specific targeting moiety (such as any one of a targeting moieties as described herein) and an effector moiety, e.g., epigenetic modifying agent, wherein a site-specific targeting moiety targets a disrupting agent to a target anchor sequence but not to at least one non-target anchor sequence. In other embodiments, the targeting moiety targets the disrupting agent to a genomic sequence element associated with a target eRNA (or a genomic complex (e.g., ASMC) comprising the target eRNA). An epigenetic modifying agent can be any one of or any combination of epigenetic modifying agents as disclosed herein.

In some embodiments, for example, fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be guided to specific DNA sites by one or more RNA sequences (sgRNA) to modulate activity and/or expression of one or more target nucleic acids sequences (e.g., to methylate or demethylate a DNA sequence).

In some embodiments, fusion of a dCas9 with all or a portion of one or more effector domains of an epigenetic modifying agent (such as a DNA methylase or enzyme with a role in DNA demethylation) creates a chimeric protein that is useful in methods provided by the present disclosure. Accordingly, for example, in some embodiments, a nucleic acid encoding a dCas9-methylase fusion in combination with a site-specific gRNA or antisense DNA oligonucleotide that targets a fusion to a genomic complex component (such as a transcription factor, ncRNA (e.g., eRNA), CTCF binding motif, etc.), may together decrease affinity or ability of a component of a genomic complex (e.g., ASMC) to interact with a particular genomic sequence. In some embodiments, a nucleic acid encoding a dCas9-enzyme fusion in combination with a site-specific gRNA or antisense DNA oligonucleotide that targets a fusion to a genomic complex component (such as a transcription factor, ncRNA (e.g., eRNA), CTCF binding motif, etc.), may together increase affinity or ability of a component of a genomic complex (e.g., ASMC) to interact with a particular genomic sequence.

In some embodiments, all or a portion of one or more methylase, or enzyme with a role in DNA demethylation, effector domains are fused with an inactive nuclease, e.g., dCas9. In some embodiments, 1, 2, 3, 4, 5, 6, 7, 8, 9, 10, 11, 12, 13, 14, 15, 16, 17, 18, 19, 20, or more methylase, or enzyme with a role in DNA demethylation, effector domains (all or a biologically active portion) are fused with dCas9. Chimeric proteins as described herein may also comprise a linker, e.g., an amino acid linker. In some embodiments, a linker comprises 2 or more amino acids, e.g., one or more GS sequences. In some embodiment, fusion of Cas9 (e.g., dCas9) with two or more effector domains (e.g., of a DNA methylase or enzyme with a role in DNA demethylation) comprises one or more interspersed linkers (e.g., GS linkers) between domains. In some aspects, dCas9 is fused with 2-5 effector domains with interspersed linkers.

In embodiments, compositions and/or methods of the present disclosure may comprise a gRNA that specifically targets a sequence or component of a genomic complex (e.g., ASMC) (e.g. CTCF binding motif, ncRNA/eRNA, transcription factor, transcription regulator, etc.). In some embodiments, the sequence or component is associated with a particular type of gene or sequence, which may be associated with one or more diseases, disorders and/or conditions.

Epigenetic modifying agents useful in provided methods and/or compositions include agents that affect, e.g., DNA methylation, histone acetylation, and RNA-associated silencing. In some embodiments, methods provided herein may involve sequence-specific targeting of an epigenetic enzyme (e.g., an enzyme that generates or removes epigenetic marks, e.g., acetylation and/or methylation). In some embodiments, exemplary epigenetic enzymes that can be targeted to an anchor sequence using the CRISPR methods described herein include DNA methylases (e.g., DNMT3a, DNMT3b, DNMTL), enzymes with a role in DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an epigenetic modifying agent useful herein comprises a construct described in Koferle et al. Genome Medicine 7.59 (2015):1-3 (e.g., at Table 1), incorporated herein by reference.

Exemplary dCas9 fusion methods and compositions that are adaptable to methods and/or compositions of the present disclosure are known and are described, e.g., in Kearns et al., Functional annotation of native enhancers with a Cas9-histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067.

In some embodiments, compositions and methods are described herein for reversibly disrupting a genomic complex, e.g., an anchor sequence-mediated conjunction, characterized by an integrity index. In some embodiments, for example, disruption may transiently modulate transcription, e.g., a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, compositions and/or methods provided herein may irreversibly disrupt a genomic complex, e.g., an anchor sequence-mediated conjunction, characterized by an integrity index.

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### EXAMPLES

### Example 1: Calculating Specificity Index (SpecInd) in a Panel of Cell Lines

As used in these Examples, loop and genomic complex are used interchangeably.

Formula 1 describes how common or unique a loop is among cell types. For instance, if a loop is present in one out of ten cell types assayed, the specificity index (SpecInd) of the loop would be 0.1, whereas if the loop were present in nine of the ten cell types assayed, the SpecInd of the loop would be 0.9. In some situations, it is advantageous to target a loop that is rare or unique among cell types (e.g., having a SpecInd of less than 0.5), in order to avoid effects in off-target tissues.

Formula 1:

$${SpecInd}_{i} = \frac{\# of cell lines where genomic complex i is present}{Total \# of cell lines}$$

Presence or absence of a given loop is determined by using an experimental technique such as ChIA-PET, HiChIP, HiC, 4C-seq, or 3C.

In this example, SpecInd was calculated using ChIA-PET across 10 cell lines. Cohesin ChIA-PET datasets from 10 cells lines generated by the ENCODE consortium (https://www.encodeproject.org/) were downloaded. The list of cell types and the accession numbers for the datasets are listed in Table 2 below:

TABLE 2

| Cell Line | Accession # |
|---|---|
| ARPE19 | ENCSR110JOO |
| Endothelium | ENCSR668RDP |
| Fibroblast | ENCSR732QOH |
| Gm12878 | ENCSR981FNA |
| H9 | ENCSR478BMT |
| Hepatocyte | ENCSR381DCY |
| HepG2 | ENCSR146FPM |
| Jurkat | ENCSR361AYD |
| K562 | ENCSR338WUS |
| MCF7 | ENCSR255XYX |

The data were processed using a custom pipeline based on the ChIA-PET2 software as described in Li et al. ChIA-PET2: a versatile and flexible pipeline for ChIA-PET data analysis (2017). Nucleic Acids Research 45(1):e4. Briefly, the pipeline consists of the following steps:

### 1. Alignment:

a. For each lane of sequencing data, the paired raw sequencing reads were aligned independently using bwa.
b. BWA output was converted to a BAM file using samtools (Samtools Organization. Samtools (2019), https://github.com/samtools/samtools).
c. Aligned reads were sorted by read name using the Picard SortSam command (Broad Institute. Picard (2019), https://broadinstitute.github.io/picard/).
   2. Making a BEDPE file with unique paired end tags (PETs):
a. The two independently aligned and sorted read BAM files were passed to the buildBedpe command of ChIAPET2 with the following parameters: mapq cutoff 30, threads 4, keep_seq 0. The output from this step is a BEDPE file.
b. BEDPE files from multiple lanes were combined using the Unix “cat” command.
c. Duplicate PETs were removed using the “rmdup” command from ChIAPET2.
   3. Peak calling:
a. BEDPE file was converted into a tags file for peak calling, Tags were sorted using the Unix “sort command”.
b. MACS2 (https://github.com/taoliu/MACS) was used to call peaks using the sorted tags file.
c. Peaks were expanded 500 bp in either direction using the bedtools “slopBed” command.
d. Sequencing coverage (“peak depth”) at each peak was computer using the bedtools “coverageBed” command from bedtools.
   4. PET clustering/loop calling:
a. The BEDPE file from step 2c and the peak depth file from step 3c were passed to the “pairToBed” command from bedtools to create a BEDPE file filtered for PETs between called peaks.
b. PETs were clustered by peak pairs using the “bedpe2Interaction” command from ChIA-PET2. This command generates two files containing intra- and inter-chromosomal PET clusters. Each file has one row per peak pair with the peak depth at each peak and number of PETs between that pair of peaks, representing an individual loop call.
   5. Loop significance calling and filtering:
a. Loop significance was calculated using the MICC2.R script provided as part of the ChIA-PET2 software. This command uses a slightly modified version of the MICC algorithm (He et al., MICC: an R package for identifying chromatin interactions from ChIA-PET data (2015). Bioinformatics 31(23):3832-4.) to examine the files from step 4b and compute a p-value and FDR q-value for a loop call between each pair of peaks.
b. A custom R script was used to filter the MICC output to include only peaks with an empirically defined FDR qvalue threshold (either 0.05 or 0.1) and an empirically determined threshold for the number of PETs supporting the loop (either 2, 3, or 5). The empirical thresholds were used to keep the number of called loops comparable across the different experiments, as the loop calling and significance calling are quite sensitive to the sequencing quality and depth of each experiment. The q-value and PET thresholds for each cell type were chosen such that about 70000 significant loops were called in each cell type, using the rationale that there was no biological reason for widely varying numbers of cohesion mediated loops across cell types. The thresholds chosen and the specific number of loops in each cell line are listed in Table 3 below:

TABLE 3
List of cell types with thresholds for
loop calling and number of called loops

| | q-value | PET | # of |
|---|---|---|---|
| Cell Line | threshold | threshold | loops |
| ARPE19 | 0.1 | 2 | 73008 |
| Endothelium | 0.05 | 3 | 72397 |
| Fibroblast | 0.05 | 4 | 75688 |
| Gm12878 | 0.05 | 8 | 71639 |
| H9 | 0.05 | 2 | 69760 |
| Hepatocyte | 0.05 | 3 | 72042 |
| HepG2 | 0.05 | 2 | 81271 |
| Jurkat | 0.15 | 2 | 60158 |
| K562 | 0.05 | 3 | 70706 |
| MCF7 | 0.05 | 2 | 73952 |

This filtered list of loops was used for the specificity index calculation using Formula 1. The total number of cell lines was 10.
Representative loops with a full range of specificity indices are listed in Table 4. Column 1 shows the position of left anchor sequence. Column 2 shows the position of right anchor sequence. Columns 3-12 show the cell type in which presence of the ASMC was measured. Column 3 shows ARPE19. Column 4 shows Endothelium. Column 5 shows Fibroblast. Column 6 shows Gm12878. Column 7 shows H9. Column 8 shows Hepatocyte. Column 9 shows HepG2. Column 10 shows Jurkat. Column 11 shows K562. Column 12 shows MCF7. Column 13 shows the loop count (number of cell lines tested having the ASMC). Column 14 shows the Specificity Index (SpecInd). Row 15, shows the gene list.

TABLE 4

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| chr5: 179245090- | chr5: 179333181- | 0 | 0 | 0 | 1 | 0 | 0 | 0 | 0 | 0 | 0 | 1 | 0.1 | C5orf45, |
| 179250204 | 179336700 | | | | | | | | | | | | | CTC-241N9.1, |
| | | | | | | | | | | | | | | TBC1D9B |
| chr20: 42859358- | chr20: 42938111- | 0 | 0 | 0 | 0 | 0 | 0 | 1 | 0 | 0 | 0 | 1 | 0.1 | GDAP1L1 |
| 42861331 | 42941078 | | | | | | | | | | | | | |
| chr8: 22906785- | chr8: 22940222- | 0 | 1 | 0 | 0 | 0 | 0 | 0 | 0 | 0 | 0 | 1 | 0.1 | TNFRSF10B |
| 22909154 | 22943100 | | | | | | | | | | | | | |
| chr20: 46121256- | chr20: 46210884- | 0 | 0 | 0 | 0 | 0 | 0 | 0 | 0 | 0 | 1 | 1 | 0.1 | NCOA3 |
| 46124167 | 46215274 | | | | | | | | | | | | | |
| chr1: 44028950- | chr1: 44113515- | 0 | 0 | 0 | 0 | 0 | 1 | 0 | 0 | 0 | 0 | 1 | 0.1 | PTPRF |
| 44032540 | 44118001 | | | | | | | | | | | | | |
| chr3: 194307160- | chr3: 194385001- | 0 | 1 | 0 | 0 | 0 | 0 | 0 | 0 | 0 | 0 | 1 | 0.1 | TMEM44 |
| 194310280 | 194386774 | | | | | | | | | | | | | |
| chr10: 72423394- | chr10: 72517460- | 1 | 0 | 0 | 0 | 0 | 0 | 0 | 0 | 0 | 0 | 1 | 0.1 | ADAMTS14 |
| 72428036 | 72522079 | | | | | | | | | | | | | |
| chr11: 65678141- | chr11: 65755188- | 0 | 0 | 0 | 1 | 0 | 0 | 0 | 0 | 0 | 0 | 1 | 0.1 | C11orf68, |
| 65681167 | 65757988 | | | | | | | | | | | | | DRAP1, |
| | | | | | | | | | | | | | | SART1 |
| chr19: 45272736- | chr19: 45359429- | 0 | 0 | 0 | 0 | 0 | 0 | 1 | 0 | 0 | 0 | 1 | 0.1 | CBLC, |
| 45275737 | 45363326 | | | | | | | | | | | | | BCAM, |
| | | | | | | | | | | | | | | PVRL2 |
| chr11: 57055443- | chr11: 57100199- | 0 | 1 | 0 | 0 | 0 | 0 | 0 | 0 | 0 | 0 | 1 | 0.1 | TNKS1BP1 |
| 57057638 | 57104534 | | | | | | | | | | | | | |
| chr19: 53589446- | chr19: 53660038- | 0 | 1 | 0 | 0 | 0 | 0 | 0 | 0 | 0 | 0 | 1 | 0.1 | ZNF160, |
| 53592201 | 53663222 | | | | | | | | | | | | | ZNF415 |
| chr4: 38819455- | chr4: 38856721- | 0 | 0 | 0 | 1 | 0 | 0 | 0 | 0 | 0 | 0 | 1 | 0.1 | TLR6 |
| 38823034 | 38860625 | | | | | | | | | | | | | |
| chr6: 13613056- | chr6: 13688107- | 0 | 0 | 0 | 0 | 0 | 0 | 0 | 0 | 1 | 0 | 1 | 0.1 | AL441883.1, |
| 13618174 | 13690944 | | | | | | | | | | | | | RANBP9 |
| chr2: 20849566- | chr2: 20874327- | 0 | 0 | 0 | 0 | 0 | 0 | 0 | 0 | 0 | 1 | 1 | 0.1 | GDF7 |
| 20852282 | 20878142 | | | | | | | | | | | | | |
| chr5: 10303316- | chr5: 10317484- | 0 | 0 | 0 | 0 | 0 | 0 | 0 | 0 | 1 | 0 | 1 | 0.1 | CMBL |
| 10306622 | 10321766 | | | | | | | | | | | | | |
| chr8: 38322055- | chr8: 38387476- | 0 | 0 | 0 | 0 | 1 | 0 | 0 | 0 | 0 | 0 | 1 | 0.1 | C8orf86 |
| 38328444 | 38389771 | | | | | | | | | | | | | |
| chr1: 207127008- | chr1: 207154169- | 0 | 0 | 0 | 1 | 0 | 0 | 0 | 0 | 0 | 0 | 1 | 0.1 | FCAMR |
| 207129894 | 207156686 | | | | | | | | | | | | | |
| chr1: 26742261- | chr1: 26825169- | 0 | 1 | 0 | 0 | 0 | 0 | 0 | 0 | 0 | 0 | 1 | 0.1 | DHDDS, |
| 26746384 | 26828785 | | | | | | | | | | | | | HMGN2 |
| chr22: 42217645- | chr22: 42257595- | 0 | 0 | 0 | 1 | 0 | 0 | 0 | 0 | 0 | 0 | 1 | 0.1 | SREBF2 |
| 42220369 | 42259982 | | | | | | | | | | | | | |
| chr19: 47342300- | chr19: 47362954- | 0 | 0 | 0 | 0 | 0 | 1 | 0 | 0 | 0 | 0 | 1 | 0.1 | AP2S1 |
| 47345730 | 47367943 | | | | | | | | | | | | | |
| chr13: 43069491- | chr13: 43147604- | 0 | 0 | 0 | 1 | 0 | 0 | 0 | 0 | 0 | 0 | 1 | 0.1 | TNFSF11 |
| 43075042 | 43150611 | | | | | | | | | | | | | |
| chr12: 52556495- | chr12: 52623181- | 0 | 0 | 1 | 0 | 0 | 0 | 0 | 0 | 0 | 0 | 1 | 0.1 | KRT80 |
| 52559469 | 52627670 | | | | | | | | | | | | | |
| chr5: 150457270- | chr5: 150503337- | 0 | 0 | 0 | 0 | 0 | 0 | 1 | 0 | 0 | 0 | 1 | 0.1 | TNIP1 |
| 150462638 | 150507279 | | | | | | | | | | | | | |
| chr1: 234657785- | chr1: 234745557- | 0 | 0 | 0 | 0 | 0 | 0 | 0 | 1 | 0 | 0 | 1 | 0.1 | IRF2BP2 |
| 234660437 | 234749889 | | | | | | | | | | | | | |
| chr1: 93248613- | chr1: 93323421- | 0 | 0 | 0 | 0 | 0 | 0 | 0 | 0 | 1 | 0 | 1 | 0.1 | EVI5, |
| 93252638 | 93326073 | | | | | | | | | | | | | RPL5 |
| chr20: 55955888- | chr20: 55989483- | 0 | 0 | 0 | 0 | 0 | 0 | 0 | 0 | 1 | 0 | 1 | 0.1 | RBM38 |
| 55958467 | 55991646 | | | | | | | | | | | | | |
| chr11: 47661852- | chr11: 47744573- | 0 | 0 | 0 | 0 | 0 | 0 | 1 | 0 | 0 | 0 | 1 | 0.1 | AGBL2 |
| 47665721 | 47746581 | | | | | | | | | | | | | |
| chr8: 128943146- | chr8: 128979487- | 0 | 1 | 0 | 0 | 0 | 0 | 0 | 0 | 0 | 0 | 1 | 0.1 | TMEM75 |
| 128945061 | 128983819 | | | | | | | | | | | | | |
| chr14: 90082579- | chr14: 90146133- | 0 | 0 | 0 | 1 | 0 | 0 | 0 | 0 | 0 | 0 | 1 | 0.1 | RP11- |
| 90088306 | 90149099 | | | | | | | | | | | | | 944C7.1 |
| chr16: 81483938- | chr16: 81575425- | 0 | 0 | 0 | 0 | 0 | 0 | 1 | 0 | 0 | 0 | 1 | 0.1 | CMIP |
| 81486809 | 81578989 | | | | | | | | | | | | | |
| chr2: 73227531- | chr2: 73311516- | 0 | 0 | 0 | 0 | 0 | 0 | 1 | 0 | 0 | 0 | 1 | 0.1 | SFXN5 |
| 73229324 | 73314392 | | | | | | | | | | | | | |
| chr9: 139928648- | chr9: 139957536- | 0 | 0 | 0 | 0 | 0 | 0 | 0 | 0 | 0 | 1 | 1 | 0.1 | NPDC1, |
| 139931615 | 139959790 | | | | | | | | | | | | | ENTPD2 |
| chr2: 219080474- | chr2: 219134009- | 0 | 0 | 0 | 0 | 0 | 1 | 1 | 0 | 0 | 0 | 2 | 0.2 | GPBAR1 |
| 219085776 | 219138696 | | | | | | | | | | | | | |
| chr11: 18784618- | chr11: 18823254- | 1 | 1 | 0 | 0 | 0 | 0 | 0 | 0 | 0 | 0 | 2 | 0.2 | PTPN5 |
| 18786856 | 18825781 | | | | | | | | | | | | | |
| chr6: 42930846- | chr6: 43025960- | 0 | 0 | 0 | 0 | 0 | 0 | 1 | 0 | 1 | 0 | 2 | 0.2 | PEX6, |
| 42932897 | 43029876 | | | | | | | | | | | | | PPP2R5D, |
| | | | | | | | | | | | | | | MEA1, |
| | | | | | | | | | | | | | | KLHDC3, |
| | | | | | | | | | | | | | | RRP36, |
| | | | | | | | | | | | | | | CUL7 |
| chr17: 72301218- | chr17: 72363025- | 0 | 1 | 0 | 0 | 0 | 0 | 0 | 0 | 1 | 0 | 2 | 0.2 | KIF19, |
| 72303157 | 72366272 | | | | | | | | | | | | | AC103809.2, |
| | | | | | | | | | | | | | | BTBD17 |
| chr5: 140854310- | chr5: 140939712- | 0 | 0 | 1 | 0 | 0 | 1 | 0 | 0 | 0 | 0 | 2 | 0.2 | PCDHGC4, |
| 140858713 | 140941523 | | | | | | | | | | | | | PCDHGC5 |
| chr11: 72850948- | chr11: 72946914- | 1 | 1 | 0 | 0 | 0 | 0 | 0 | 0 | 0 | 0 | 2 | 0.2 | P2RY2 |
| 72855362 | 72948944 | | | | | | | | | | | | | |
| chr1: 85132582- | chr1: 85188899- | 0 | 0 | 0 | 1 | 0 | 0 | 0 | 0 | 1 | 0 | 2 | 0.2 | SSX2IP |
| 85135244 | 85191427 | | | | | | | | | | | | | |
| chr15: 93186952- | chr15: 93223978- | 0 | 0 | 0 | 0 | 1 | 0 | 0 | 0 | 0 | 1 | 2 | 0.2 | FAM174B |
| 93192461 | 93228069 | | | | | | | | | | | | | |
| chr9: 124093338- | chr9: 124159909- | 0 | 1 | 1 | 0 | 0 | 0 | 0 | 0 | 0 | 0 | 2 | 0.2 | AL161784.1, |
| 124095561 | 124163742 | | | | | | | | | | | | | STOM |
| chr12: 52974765- | chr12: 53060614- | 0 | 0 | 0 | 0 | 0 | 0 | 0 | 1 | 0 | 1 | 2 | 0.2 | KRT72, |
| 52978573 | 53062563 | | | | | | | | | | | | | KRT73, |
| | | | | | | | | | | | | | | KRT2 |
| chr17: 36503946- | chr17: 36598443- | 0 | 0 | 0 | 0 | 0 | 1 | 0 | 1 | 0 | 0 | 2 | 0.2 | ARHGAP23 |
| 36508260 | 36601623 | | | | | | | | | | | | | |
| chr17: 43798455- | chr17: 43889051- | 0 | 1 | 0 | 0 | 1 | 0 | 0 | 0 | 0 | 0 | 2 | 0.2 | CRHR1 |
| 43800759 | 43891873 | | | | | | | | | | | | | |
| chr20: 39617932- | chr20: 39675556- | 0 | 0 | 0 | 1 | 0 | 0 | 0 | 0 | 1 | 0 | 2 | 0.2 | TOP1 |
| 39621013 | 39681473 | | | | | | | | | | | | | |
| chr11: 34620249- | chr11: 34671312- | 0 | 0 | 0 | 0 | 0 | 0 | 1 | 0 | 1 | 0 | 2 | 0.2 | EHF |
| 34626321 | 34678702 | | | | | | | | | | | | | |
| chr17: 72205504- | chr17: 72291439- | 0 | 0 | 0 | 0 | 0 | 1 | 0 | 0 | 1 | 0 | 2 | 0.2 | TTYH2, |
| 72210349 | 72293768 | | | | | | | | | | | | | DNAI2 |
| chr20: 43157958- | chr20: 43228846- | 0 | 1 | 1 | 0 | 0 | 0 | 0 | 0 | 0 | 0 | 2 | 0.2 | PKIG |
| 43162957 | 43232776 | | | | | | | | | | | | | |
| chr11: 71129383- | chr11: 71162523- | 0 | 0 | 0 | 1 | 0 | 0 | 0 | 1 | 0 | 0 | 2 | 0.2 | DHCR7 |
| 71133363 | 71166911 | | | | | | | | | | | | | |
| chr1: 26631839- | chr1: 26646111- | 0 | 0 | 0 | 1 | 0 | 0 | 0 | 0 | 1 | 0 | 2 | 0.2 | CD52, |
| 26635871 | 26649537 | | | | | | | | | | | | | UBXN11 |
| chr11: 65306689- | chr11: 65391980- | 0 | 1 | 0 | 0 | 0 | 0 | 0 | 0 | 1 | 0 | 2 | 0.2 | LTBP3, |
| 65310149 | 65394955 | | | | | | | | | | | | | SSSCA1, |
| | | | | | | | | | | | | | | FAM89B, |
| | | | | | | | | | | | | | | EHBP1L1, |
| | | | | | | | | | | | | | | AP001362.1, |
| | | | | | | | | | | | | | | KCNK7, |
| | | | | | | | | | | | | | | MAP3K11, |
| | | | | | | | | | | | | | | PCNXL3 |
| chr9: 21432225- | chr9: 21454011- | 0 | 0 | 1 | 1 | 0 | 0 | 0 | 0 | 0 | 0 | 2 | 0.2 | IFNA1 |
| 21436184 | 21456566 | | | | | | | | | | | | | |
| chr14: 61977943- | chr14: 62025711- | 0 | 0 | 1 | 0 | 0 | 0 | 0 | 0 | 0 | 1 | 2 | 0.2 | RP11- |
| 61985031 | 62037474 | | | | | | | | | | | | | 47I22.4 |
| chr2: 27716671- | chr2: 27804831- | 0 | 1 | 0 | 0 | 0 | 0 | 1 | 0 | 0 | 0 | 2 | 0.2 | AC109829.1, |
| 27720613 | 27808378 | | | | | | | | | | | | | C2orf16 |
| chr12: 53373503- | chr12: 53439490- | 0 | 0 | 0 | 0 | 0 | 1 | 0 | 0 | 0 | 1 | 2 | 0.2 | EIF4B |
| 53375930 | 53446051 | | | | | | | | | | | | | |
| chr11: 118480624- | chr11: 118559693- | 0 | 0 | 0 | 0 | 1 | 1 | 0 | 0 | 0 | 0 | 2 | 0.2 | PHLDB1, |
| 118482812 | 118562270 | | | | | | | | | | | | | TREH |
| chr1: 160803998- | chr1: 160845966- | 0 | 0 | 0 | 1 | 0 | 0 | 0 | 1 | 0 | 0 | 2 | 0.2 | CD244 |
| 160808429 | 160848276 | | | | | | | | | | | | | |
| chr16: 20358857- | chr16: 20393645- | 0 | 0 | 0 | 0 | 1 | 0 | 1 | 0 | 0 | 0 | 2 | 0.2 | UMOD |
| 20361887 | 20398532 | | | | | | | | | | | | | |
| chr19: 56027359- | chr19: 56060308- | 0 | 1 | 1 | 0 | 0 | 0 | 0 | 0 | 0 | 0 | 2 | 0.2 | SBK2, |
| 56029632 | 56062449 | | | | | | | | | | | | | SBK3 |
| chr1: 47656724- | chr1: 47696578- | 0 | 1 | 0 | 0 | 0 | 0 | 0 | 0 | 1 | 0 | 2 | 0.2 | TAL1 |
| 47660869 | 47699033 | | | | | | | | | | | | | |
| chr2: 71011294- | chr2: 71087882- | 0 | 0 | 0 | 0 | 0 | 0 | 0 | 0 | 1 | 1 | 2 | 0.2 | FIGLA, |
| 71013692 | 71090729 | | | | | | | | | | | | | CLEC4F, |
| | | | | | | | | | | | | | | CD207 |
| chr17: 7378607- | chr17: 7462838- | 1 | 0 | 0 | 0 | 0 | 1 | 0 | 0 | 0 | 0 | 2 | 0.2 | SLC35G6, |
| 7384053 | 7465763 | | | | | | | | | | | | | ZBTB4, |
| | | | | | | | | | | | | | | POLR2A, |
| | | | | | | | | | | | | | | TNFSF12, |
| | | | | | | | | | | | | | | TNFSF12- |
| | | | | | | | | | | | | | | TNFSF13, |
| | | | | | | | | | | | | | | TNFSF12, |
| | | | | | | | | | | | | | | TNFSF13 |
| chr17: 47267925- | chr17: 47294901- | 0 | 0 | 0 | 1 | 0 | 0 | 0 | 1 | 0 | 0 | 2 | 0.2 | GNGT2, |
| 47272252 | 47297811 | | | | | | | | | | | | | ABI3, |
| | | | | | | | | | | | | | | GNGT2 |
| chr2: 73295778- | chr2: 73389385- | 0 | 1 | 0 | 0 | 0 | 0 | 0 | 0 | 1 | 0 | 2 | 0.2 | RAB11FIP5 |
| 73301077 | 73392636 | | | | | | | | | | | | | |
| chr17: 41115273- | chr17: 41148860- | 0 | 1 | 0 | 1 | 0 | 0 | 0 | 0 | 0 | 0 | 2 | 0.2 | PTGES3L- |
| 41118312 | 41152210 | | | | | | | | | | | | | AARSD1, |
| | | | | | | | | | | | | | | PTGES3L, |
| | | | | | | | | | | | | | | PTGES3L- |
| | | | | | | | | | | | | | | AARSD1, |
| | | | | | | | | | | | | | | PTGES3L, |
| | | | | | | | | | | | | | | RUNDC1 |
| chr10: 91052368- | chr10: 91109955- | 0 | 0 | 1 | 1 | 0 | 0 | 0 | 0 | 0 | 0 | 2 | 0.2 | IFIT2, |
| 91054841 | 91113340 | | | | | | | | | | | | | IFIT3 |
| chr11: 130013382- | chr11: 130081878- | 0 | 0 | 0 | 0 | 0 | 1 | 1 | 0 | 0 | 0 | 2 | 0.2 | ST14 |
| 130017629 | 130085077 | | | | | | | | | | | | | |
| chr19: 2949498- | chr19: 3034087- | 0 | 1 | 0 | 0 | 0 | 1 | 1 | 0 | 0 | 0 | 3 | 0.3 | TLE6, |
| 2953644 | 3037586 | | | | | | | | | | | | | TLE2 |
| chrX: 152818419- | chrX: 152873465- | 0 | 0 | 0 | 0 | 0 | 1 | 0 | 0 | 1 | 1 | 3 | 0.3 | FAM58A |
| 152821836 | 152877153 | | | | | | | | | | | | | |
| chr17: 71250721- | chr17: 71297891- | 0 | 0 | 1 | 0 | 0 | 1 | 0 | 0 | 1 | 0 | 3 | 0.3 | CPSF4L |
| 71254222 | 71302365 | | | | | | | | | | | | | |
| chr20: 60637824- | chr20: 60709390- | 0 | 0 | 0 | 0 | 0 | 1 | 1 | 0 | 0 | 1 | 3 | 0.3 | LSM14B |
| 60643853 | 60713275 | | | | | | | | | | | | | |
| chr16: 67960069- | chr16: 67974884- | 0 | 1 | 0 | 0 | 0 | 0 | 1 | 0 | 1 | 0 | 3 | 0.3 | CTRL, |
| 67964118 | 67978939 | | | | | | | | | | | | | PSMB10 |
| chr19: 18595189- | chr19: 18681280- | 0 | 0 | 0 | 0 | 0 | 0 | 1 | 0 | 1 | 1 | 3 | 0.3 | ELL, |
| 18600931 | 18684544 | | | | | | | | | | | | | FKBP8, |
| | | | | | | | | | | | | | | KXD1 |
| chr19: 47102921- | chr19: 47140611- | 0 | 0 | 0 | 1 | 0 | 1 | 0 | 1 | 0 | 0 | 3 | 0.3 | PTGIR, |
| 47107381 | 47143484 | | | | | | | | | | | | | GNG8 |
| chr11: 413998- | chr11: 447368- | 0 | 1 | 0 | 0 | 1 | 1 | 0 | 0 | 0 | 0 | 3 | 0.3 | ANO9 |
| 418452 | 452153 | | | | | | | | | | | | | |
| chr17: 75400453- | chr17: 75489706- | 0 | 1 | 0 | 1 | 0 | 0 | 0 | 1 | 0 | 0 | 3 | 0.3 | 43717 |
| 75403037 | 75492589 | | | | | | | | | | | | | |
| chr1: 167034620- | chr1: 167072117- | 0 | 0 | 0 | 1 | 1 | 0 | 0 | 1 | 0 | 0 | 3 | 0.3 | GPA33, |
| 167037527 | 167076094 | | | | | | | | | | | | | DUSP27 |
| chr9: 139961315- | chr9: 140007328- | 0 | 0 | 0 | 1 | 0 | 0 | 1 | 0 | 1 | 0 | 3 | 0.3 | SAPCD2, |
| 139963780 | 140018701 | | | | | | | | | | | | | UAP1L1, |
| | | | | | | | | | | | | | | AL807752.1, |
| | | | | | | | | | | | | | | MAN1B1 |
| chr6: 134494208- | chr6: 134566628- | 0 | 0 | 0 | 1 | 0 | 0 | 1 | 1 | 0 | 0 | 3 | 0.3 | SGK1 |
| 134497186 | 134572255 | | | | | | | | | | | | | |
| chr11: 3041323- | chr11: 3111758- | 1 | 0 | 1 | 1 | 0 | 0 | 0 | 0 | 0 | 0 | 3 | 0.3 | CARS |
| 3045076 | 3114657 | | | | | | | | | | | | | |
| chr8: 67349766- | chr8: 67444700- | 0 | 0 | 1 | 1 | 0 | 0 | 0 | 1 | 0 | 0 | 3 | 0.3 | C8orf46 |
| 67352857 | 67449098 | | | | | | | | | | | | | |
| chr12: 100741376- | chr12: 100797514- | 0 | 1 | 0 | 0 | 1 | 0 | 1 | 0 | 0 | 0 | 3 | 0.3 | SLC17A8 |
| 100744283 | 100799990 | | | | | | | | | | | | | |
| chr18: 52224285- | chr18: 52312686- | 0 | 1 | 0 | 0 | 1 | 1 | 0 | 0 | 0 | 0 | 3 | 0.3 | DYNAP |
| 52227693 | 52315691 | | | | | | | | | | | | | |
| chr17: 73838964- | chr17: 73900164- | 0 | 0 | 0 | 1 | 0 | 0 | 1 | 0 | 1 | 0 | 3 | 0.3 | WBP2, |
| 73846789 | 73902721 | | | | | | | | | | | | | TRIM47, |
| | | | | | | | | | | | | | | TRIM65 |
| chr12: 50231657- | chr12: 50259841- | 0 | 1 | 0 | 0 | 0 | 0 | 1 | 0 | 0 | 1 | 3 | 0.3 | BCDIN3D |
| 50234122 | 50263923 | | | | | | | | | | | | | |
| chr20: 288605- | chr20: 310016- | 0 | 0 | 1 | 0 | 0 | 1 | 1 | 0 | 0 | 0 | 3 | 0.3 | SOX12 |
| 292165 | 312575 | | | | | | | | | | | | | |
| chr5: 139933655- | chr5: 140025881- | 0 | 0 | 0 | 0 | 0 | 1 | 1 | 0 | 1 | 0 | 3 | 0.3 | APBB3, |
| 139938431 | 140028863 | | | | | | | | | | | | | SLC35A4, |
| | | | | | | | | | | | | | | APBB3, |
| | | | | | | | | | | | | | | SLC35A4, |
| | | | | | | | | | | | | | | CD14, |
| | | | | | | | | | | | | | | TMCO6 |
| chr13: 100093671- | chr13: 100156890- | 0 | 1 | 1 | 1 | 0 | 0 | 0 | 0 | 0 | 0 | 3 | 0.3 | TM9SF2 |
| 100096714 | 100159696 | | | | | | | | | | | | | |
| chr1: 21587031- | chr1: 21669262- | 0 | 1 | 1 | 0 | 0 | 1 | 0 | 0 | 0 | 0 | 3 | 0.3 | ECE1 |
| 21590168 | 21673644 | | | | | | | | | | | | | |
| chr6: 31773095- | chr6: 31797078- | 0 | 1 | 0 | 0 | 0 | 1 | 0 | 0 | 1 | 0 | 3 | 0.3 | HSPA1L, |
| 31776124 | 31800315 | | | | | | | | | | | | | HSPA1A, |
| | | | | | | | | | | | | | | HSPA1L, |
| | | | | | | | | | | | | | | HSPA1B |
| chr19: 49401878- | chr19: 49465017- | 0 | 1 | 0 | 1 | 0 | 0 | 0 | 0 | 1 | 0 | 3 | 0.3 | DHDH, |
| 49405257 | 49469874 | | | | | | | | | | | | | BAX |
| chr19: 42942083- | chr19: 43031907- | 0 | 0 | 0 | 1 | 1 | 0 | 0 | 1 | 0 | 0 | 3 | 0.3 | CXCL17 |
| 42946444 | 43034720 | | | | | | | | | | | | | |
| chr10: 72137276- | chr10: 72219789- | 0 | 0 | 0 | 1 | 1 | 0 | 0 | 0 | 1 | 0 | 3 | 0.3 | LRRC20, |
| 72140816 | 72223246 | | | | | | | | | | | | | EIF4EBP2, |
| | | | | | | | | | | | | | | AC022532.1, |
| | | | | | | | | | | | | | | NODAL |
| chr21: 46219226- | chr21: 46254473- | 0 | 0 | 0 | 0 | 0 | 0 | 1 | 0 | 1 | 1 | 3 | 0.3 | SUMO3 |
| 46224564 | 46257951 | | | | | | | | | | | | | |
| chr20: 17510311- | chr20: 17548718- | 0 | 1 | 0 | 0 | 0 | 1 | 0 | 0 | 0 | 1 | 3 | 0.3 | BFSP1 |
| 17513152 | 17553594 | | | | | | | | | | | | | |
| chr12: 104679067- | chr12: 104750803- | 0 | 0 | 0 | 1 | 1 | 0 | 0 | 0 | 1 | 0 | 3 | 0.3 | TXNRD1, |
| 104684091 | 104753749 | | | | | | | | | | | | | EID3, |
| | | | | | | | | | | | | | | TXNRD1 |
| chr14: 75984194- | chr14: 76025679- | 0 | 1 | 0 | 1 | 0 | 0 | 1 | 0 | 0 | 0 | 3 | 0.3 | BATF |
| 75986875 | 76029095 | | | | | | | | | | | | | |
| chr11: 118491088- | chr11: 118528963- | 1 | 1 | 0 | 0 | 0 | 1 | 0 | 0 | 0 | 0 | 3 | 0.3 | PHLDB1 |
| 118494863 | 118531718 | | | | | | | | | | | | | |
| chr10: 111715219- | chr10: 111774987- | 0 | 0 | 1 | 1 | 0 | 0 | 1 | 0 | 0 | 0 | 3 | 0.3 | ADD3 |
| 111718170 | 111777788 | | | | | | | | | | | | | |
| chr20: 25519132- | chr20: 25602242- | 0 | 1 | 1 | 0 | 0 | 0 | 1 | 0 | 0 | 0 | 3 | 0.3 | NINL |
| 25521985 | 25605948 | | | | | | | | | | | | | |
| chr19: 14151859- | chr19: 14182072- | 0 | 1 | 0 | 1 | 0 | 0 | 0 | 0 | 1 | 0 | 3 | 0.3 | PALM3 |
| 14154949 | 14187037 | | | | | | | | | | | | | |
| chr19: 54691510- | chr19: 54711102- | 0 | 0 | 0 | 1 | 0 | 1 | 1 | 0 | 0 | 0 | 3 | 0.3 | RPS9 |
| 54696558 | 54715889 | | | | | | | | | | | | | |
| chr22: 29789606- | chr22: 29865068- | 0 | 0 | 0 | 1 | 1 | 0 | 0 | 0 | 0 | 1 | 3 | 0.3 | AP1B1, |
| 29792932 | 29867784 | | | | | | | | | | | | | RFPL1 |
| chr21: 45135929- | chr21: 45207978- | 0 | 1 | 0 | 0 | 0 | 1 | 0 | 0 | 1 | 1 | 4 | 0.4 | PDXK, |
| 45141051 | 45211101 | | | | | | | | | | | | | CSTB |
| chr1: 201682106- | chr1: 201760524- | 1 | 1 | 1 | 0 | 0 | 0 | 0 | 0 | 0 | 1 | 4 | 0.4 | NAV1 |
| 201684758 | 201765207 | | | | | | | | | | | | | |
| chr15: 93578451- | chr15: 93629797- | 0 | 0 | 0 | 0 | 1 | 1 | 0 | 1 | 0 | 1 | 4 | 0.4 | RGMA |
| 93581867 | 93633585 | | | | | | | | | | | | | |
| chr19: 6055417- | chr19: 6124643- | 0 | 1 | 1 | 0 | 0 | 1 | 1 | 0 | 0 | 0 | 4 | 0.4 | RFX2 |
| 6058907 | 6127608 | | | | | | | | | | | | | |
| chr20: 43963907- | chr20: 43990128- | 1 | 0 | 1 | 0 | 0 | 0 | 1 | 0 | 1 | 0 | 4 | 0.4 | SDC4 |
| 43968512 | 43993015 | | | | | | | | | | | | | |
| chr11: 64509281- | chr11: 64534298- | 0 | 1 | 1 | 0 | 0 | 0 | 0 | 1 | 1 | 0 | 4 | 0.4 | RASGRP2, |
| 64512429 | 64537149 | | | | | | | | | | | | | PYGM |
| chr16: 84149016- | chr16: 84207647- | 0 | 1 | 0 | 0 | 0 | 1 | 1 | 0 | 1 | 0 | 4 | 0.4 | HSDL1, |
| 84152012 | 84211508 | | | | | | | | | | | | | DNAAF1 |
| chr6: 28233542- | chr6: 28321530- | 0 | 0 | 0 | 1 | 0 | 1 | 1 | 0 | 1 | 0 | 4 | 0.4 | PGBD1, |
| 28238112 | 28325583 | | | | | | | | | | | | | ZSCAN31, |
| | | | | | | | | | | | | | | ZKSCAN3 |
| chr1: 86883974- | chr1: 86972551- | 0 | 1 | 1 | 0 | 0 | 0 | 0 | 1 | 1 | 0 | 4 | 0.4 | CLCA2, |
| 86886631 | 86975352 | | | | | | | | | | | | | CLCA1 |
| chr7: 23693033- | chr7: 23750872- | 1 | 1 | 0 | 0 | 1 | 0 | 0 | 0 | 1 | 0 | 4 | 0.4 | FAM221A, |
| 23695551 | 23754466 | | | | | | | | | | | | | STK31 |
| chr11: 63244844- | chr11: 63339379- | 0 | 0 | 1 | 1 | 0 | 0 | 0 | 1 | 1 | 0 | 4 | 0.4 | HRASLS5, |
| 63248257 | 63343488 | | | | | | | | | | | | | LGALS12, |
| | | | | | | | | | | | | | | RARRES3, |
| | | | | | | | | | | | | | | HRASLS2 |
| chr6: 158937335- | chr6: 159024786- | 1 | 1 | 1 | 0 | 0 | 0 | 0 | 0 | 1 | 0 | 4 | 0.4 | TMEM181 |
| 158941396 | 159027632 | | | | | | | | | | | | | |
| chr11: 44558769- | chr11: 44637064- | 1 | 1 | 0 | 0 | 1 | 0 | 0 | 0 | 1 | 0 | 4 | 0.4 | CD82 |
| 44561628 | 44639562 | | | | | | | | | | | | | |
| chr3: 123360302- | chr3: 123419691- | 1 | 1 | 1 | 0 | 0 | 0 | 0 | 0 | 0 | 1 | 4 | 0.4 | MYLK |
| 123362644 | 123423623 | | | | | | | | | | | | | |
| chr8: 103545967- | chr8: 103595361- | 0 | 1 | 1 | 1 | 1 | 0 | 0 | 0 | 0 | 0 | 4 | 0.4 | ODF1 |
| 103550904 | 103599561 | | | | | | | | | | | | | |
| chr19: 49647949- | chr19: 49710439- | 1 | 1 | 0 | 0 | 0 | 0 | 1 | 0 | 1 | 0 | 4 | 0.4 | HRC, |
| 49650416 | 49714558 | | | | | | | | | | | | | TRPM4 |
| chr11: 65546300- | chr11: 65623644- | 0 | 1 | 0 | 1 | 1 | 0 | 1 | 0 | 0 | 0 | 4 | 0.4 | OVOL1, |
| 65549622 | 65629309 | | | | | | | | | | | | | SNX32 |
| chr1: 152318351- | chr1: 152412315- | 1 | 0 | 1 | 1 | 0 | 0 | 0 | 1 | 0 | 0 | 4 | 0.4 | FLG2, |
| 152323088 | 152415278 | | | | | | | | | | | | | CRNN |
| chr16: 89665187- | chr16: 89706346- | 0 | 1 | 0 | 0 | 0 | 0 | 1 | 0 | 1 | 1 | 4 | 0.4 | DPEP1 |
| 89668147 | 89710292 | | | | | | | | | | | | | |
| chr17: 36060157- | chr17: 36150195- | 0 | 0 | 1 | 1 | 0 | 0 | 0 | 1 | 1 | 0 | 4 | 0.4 | HNF1B |
| 36063747 | 36153642 | | | | | | | | | | | | | |
| chr5: 140969196- | chr5: 141042961- | 1 | 0 | 0 | 1 | 0 | 1 | 0 | 1 | 0 | 0 | 4 | 0.4 | DIAPH1, |
| 140973682 | 141045850 | | | | | | | | | | | | | HDAC3, |
| | | | | | | | | | | | | | | RELL2, |
| | | | | | | | | | | | | | | FCHSD1 |
| chr6: 75974832- | chr6: 75992046- | 0 | 1 | 1 | 1 | 0 | 1 | 0 | 0 | 0 | 0 | 4 | 0.4 | TMEM30A |
| 75979068 | 75996047 | | | | | | | | | | | | | |
| chr8: 95905215- | chr8: 95958048- | 0 | 1 | 0 | 0 | 0 | 1 | 1 | 0 | 1 | 0 | 4 | 0.4 | TP53INP1 |
| 95910113 | 95963222 | | | | | | | | | | | | | |
| chr4: 4488307- | chr4: 4575836- | 1 | 1 | 0 | 1 | 0 | 1 | 0 | 0 | 0 | 0 | 4 | 0.4 | STX18 |
| 4491572 | 4578532 | | | | | | | | | | | | | |
| chr7: 107820065- | chr7: 107885767- | 1 | 1 | 1 | 0 | 0 | 0 | 0 | 0 | 1 | 0 | 4 | 0.4 | NRCAM |
| 107823503 | 107888897 | | | | | | | | | | | | | |
| chr15: 93154444- | chr15: 93223978- | 0 | 0 | 0 | 1 | 1 | 0 | 0 | 0 | 1 | 1 | 4 | 0.4 | FAM174B |
| 93157057 | 93228069 | | | | | | | | | | | | | |
| chr2: 96685721- | chr2: 96781613- | 0 | 1 | 1 | 1 | 0 | 0 | 0 | 0 | 1 | 0 | 4 | 0.4 | GPAT2 |
| 96691240 | 96784691 | | | | | | | | | | | | | |
| chr16: 68398163- | chr16: 68477561- | 0 | 1 | 0 | 1 | 1 | 0 | 0 | 0 | 0 | 1 | 4 | 0.4 | SMPD3 |
| 68404569 | 68481119 | | | | | | | | | | | | | |
| chr10: 97133066- | chr10: 97204339- | 1 | 1 | 0 | 1 | 0 | 0 | 0 | 0 | 1 | 0 | 4 | 0.4 | SORBS1 |
| 97136043 | 97207539 | | | | | | | | | | | | | |
| chr7: 143084303- | chr7: 143112142- | 0 | 1 | 1 | 0 | 1 | 0 | 0 | 0 | 1 | 0 | 4 | 0.4 | EPHA1 |
| 143090056 | 143115695 | | | | | | | | | | | | | |
| chr4: 77867768- | chr4: 77941698- | 1 | 1 | 1 | 0 | 0 | 1 | 0 | 0 | 0 | 0 | 4 | 0.4 | 43719 |
| 77873639 | 77945875 | | | | | | | | | | | | | |
| chr1: 6407114- | chr1: 6456350- | 0 | 1 | 0 | 0 | 0 | 0 | 0 | 1 | 1 | 1 | 4 | 0.4 | ACOT7 |
| 6409633 | 6458204 | | | | | | | | | | | | | |
| chr16: 67188495- | chr16: 67202676- | 0 | 1 | 1 | 0 | 0 | 0 | 1 | 0 | 1 | 0 | 4 | 0.4 | FBXL8, |
| 67191296 | 67205828 | | | | | | | | | | | | | TRADD, |
| | | | | | | | | | | | | | | HSF4 |
| chrX: 114794082- | chrX: 114875888- | 1 | 1 | 0 | 0 | 1 | 1 | 0 | 0 | 0 | 0 | 4 | 0.4 | PLS3 |
| 114799011 | 114878749 | | | | | | | | | | | | | |
| chr14: 94404743- | chr14: 94501898- | 1 | 0 | 1 | 1 | 0 | 0 | 1 | 0 | 0 | 0 | 4 | 0.4 | ASB2, |
| 94407725 | 94504293 | | | | | | | | | | | | | OTUB2 |
| chr2: 220322188- | chr2: 220384677- | 0 | 0 | 1 | 0 | 1 | 1 | 0 | 0 | 1 | 0 | 4 | 0.4 | GMPPA, |
| 220326146 | 220389017 | | | | | | | | | | | | | ASIC4 |
| chr1: 236651911- | chr1: 236693904- | 0 | 1 | 0 | 0 | 0 | 0 | 1 | 0 | 1 | 1 | 4 | 0.4 | LGALS8 |
| 236655165 | 236697043 | | | | | | | | | | | | | |
| chr1: 145037927- | chr1: 145089378- | 1 | 0 | 1 | 0 | 1 | 0 | 1 | 0 | 0 | 0 | 4 | 0.4 | PDE4DIP |
| 145043784 | 145093493 | | | | | | | | | | | | | |
| chr3: 183185561- | chr3: 183277428- | 0 | 1 | 0 | 1 | 1 | 0 | 0 | 0 | 1 | 0 | 4 | 0.4 | KLHL6 |
| 183188718 | 183279748 | | | | | | | | | | | | | |
| chr7: 150572257- | chr7: 150659076- | 0 | 1 | 0 | 0 | 1 | 0 | 0 | 1 | 1 | 0 | 4 | 0.4 | KCNH2 |
| 150575765 | 150663947 | | | | | | | | | | | | | |
| chr5: 139153865- | chr5: 139222787- | 0 | 1 | 0 | 1 | 0 | 1 | 0 | 0 | 1 | 0 | 4 | 0.4 | PSD2 |
| 139156778 | 139225439 | | | | | | | | | | | | | |
| chr1: 151734598- | chr1: 151761519- | 0 | 1 | 0 | 0 | 0 | 0 | 0 | 1 | 1 | 1 | 4 | 0.4 | OAZ3 |
| 151738169 | 151764648 | | | | | | | | | | | | | |
| chr17: 47020246- | chr17: 47089430- | 0 | 0 | 1 | 0 | 1 | 0 | 1 | 0 | 0 | 1 | 4 | 0.4 | GIP, |
| 47023343 | 47094345 | | | | | | | | | | | | | IGF2BP1 |
| chr20: 1086660- | chr20: 1164866- | 0 | 1 | 0 | 1 | 0 | 0 | 1 | 0 | 1 | 0 | 4 | 0.4 | PSMF1 |
| 1090895 | 1169551 | | | | | | | | | | | | | |
| chr19: 11544856- | chr19: 11591744- | 0 | 1 | 0 | 1 | 0 | 1 | 0 | 0 | 0 | 1 | 4 | 0.4 | ELAVL3 |
| 11547615 | 11594702 | | | | | | | | | | | | | |
| chr10: 104487653- | chr10: 104511299- | 0 | 1 | 1 | 0 | 0 | 0 | 1 | 1 | 0 | 0 | 4 | 0.4 | WBP1L |
| 104490548 | 104514715 | | | | | | | | | | | | | |
| chr16: 3143834- | chr16: 3173086- | 0 | 1 | 0 | 1 | 1 | 0 | 0 | 0 | 1 | 0 | 4 | 0.4 | ZSCAN10, |
| 3147094 | 3175607 | | | | | | | | | | | | | ZNF205 |
| chr19: 18704417- | chr19: 18782080- | 0 | 1 | 1 | 0 | 0 | 1 | 0 | 0 | 1 | 0 | 4 | 0.4 | CRLF1, |
| 18706458 | 18784573 | | | | | | | | | | | | | TMEM59L, |
| | | | | | | | | | | | | | | KLHL26 |
| chr17: 7182473- | chr17: 7209510- | 0 | 1 | 0 | 1 | 0 | 1 | 1 | 0 | 0 | 1 | 5 | 0.5 | YBX2 |
| 7185568 | 7213302 | | | | | | | | | | | | | |
| chr11: 61243576- | chr11: 61298905- | 0 | 1 | 1 | 0 | 0 | 0 | 0 | 1 | 1 | 1 | 5 | 0.5 | PPP1R32, |
| 61246959 | 61301075 | | | | | | | | | | | | | LRRC10B |
| chr22: 41797576- | chr22: 41862846- | 0 | 1 | 0 | 1 | 1 | 0 | 1 | 1 | 0 | 0 | 5 | 0.5 | TOB2 |
| 41802347 | 41866770 | | | | | | | | | | | | | |
| chr11: 64050071- | chr11: 64071044- | 0 | 1 | 0 | 0 | 0 | 1 | 1 | 1 | 0 | 1 | 5 | 0.5 | KCNK4, |
| 64054942 | 64075138 | | | | | | | | | | | | | TEX40 |
| chr19: 45561416- | chr19: 45628256- | 1 | 0 | 1 | 0 | 0 | 0 | 1 | 0 | 1 | 1 | 5 | 0.5 | ZNF296, |
| 45564330 | 45630916 | | | | | | | | | | | | | GEMIN7, |
| | | | | | | | | | | | | | | PPP1R37 |
| chr2: 45207473- | chr2: 45240238- | 1 | 1 | 1 | 0 | 1 | 1 | 0 | 0 | 0 | 0 | 5 | 0.5 | SIX2 |
| 45210690 | 45242551 | | | | | | | | | | | | | |
| chr3: 141084696- | chr3: 141172933- | 0 | 0 | 1 | 0 | 0 | 1 | 1 | 0 | 1 | 1 | 5 | 0.5 | ZBTB38 |
| 141090078 | 141175037 | | | | | | | | | | | | | |
| chr6: 26120841- | chr6: 26195028- | 0 | 1 | 0 | 1 | 0 | 1 | 1 | 0 | 1 | 0 | 5 | 0.5 | HIST1H1E, |
| 26127716 | 26200969 | | | | | | | | | | | | | HIST1H2BD, |
| | | | | | | | | | | | | | | HIST1H2BE, |
| | | | | | | | | | | | | | | HIST1H4D |
| chr8: 146010255- | chr8: 146029029- | 1 | 0 | 0 | 0 | 0 | 1 | 1 | 0 | 1 | 1 | 5 | 0.5 | RPL8, |
| 146014165 | 146031683 | | | | | | | | | | | | | ZNF517 |
| chr19: 13047753- | chr19: 13074901- | 0 | 1 | 1 | 1 | 0 | 1 | 1 | 0 | 0 | 0 | 5 | 0.5 | RAD23A, |
| 13051069 | 13077469 | | | | | | | | | | | | | GADD45 |
| | | | | | | | | | | | | | | GIP1 |
| chr17: 47047784- | chr17: 47108330- | 1 | 0 | 1 | 0 | 0 | 0 | 1 | 0 | 1 | 1 | 5 | 0.5 | IGF2BP1 |
| 47051996 | 47110909 | | | | | | | | | | | | | |
| chr16: 22087977- | chr16: 22108964- | 0 | 0 | 1 | 1 | 1 | 0 | 1 | 0 | 1 | 0 | 5 | 0.5 | VWA3A |
| 22090680 | 22111660 | | | | | | | | | | | | | |
| chr11: 72300151- | chr11: 72393719- | 0 | 1 | 0 | 1 | 1 | 1 | 0 | 0 | 1 | 0 | 5 | 0.5 | PDE2A |
| 72301986 | 72396568 | | | | | | | | | | | | | |
| chr19: 46144106- | chr19: 46194240- | 0 | 0 | 0 | 1 | 1 | 0 | 1 | 0 | 1 | 1 | 5 | 0.5 | EML2, |
| 46147899 | 46197957 | | | | | | | | | | | | | GIPR |
| chr7: 43766260- | chr7: 43847183- | 0 | 0 | 0 | 1 | 0 | 0 | 1 | 1 | 1 | 1 | 5 | 0.5 | BLVRA |
| 43772032 | 43851190 | | | | | | | | | | | | | |
| chr14: 21438170- | chr14: 21481955- | 1 | 1 | 0 | 0 | 1 | 1 | 1 | 0 | 0 | 0 | 5 | 0.5 | METTL17, |
| 21440922 | 21484461 | | | | | | | | | | | | | SLC39A2 |
| chr4: 2220398- | chr4: 2242338- | 1 | 1 | 0 | 0 | 1 | 0 | 0 | 1 | 1 | 0 | 5 | 0.5 | POLN |
| 2223223 | 2246536 | | | | | | | | | | | | | |
| chr2: 99700229- | chr2: 99794709- | 1 | 0 | 1 | 1 | 0 | 0 | 1 | 0 | 1 | 0 | 5 | 0.5 | TSGA10, |
| 99703386 | 99798860 | | | | | | | | | | | | | C2ORF15, |
| | | | | | | | | | | | | | | TSGA10, |
| | | | | | | | | | | | | | | C2ORF15, |
| | | | | | | | | | | | | | | TSGA10, |
| | | | | | | | | | | | | | | LIPT1, |
| | | | | | | | | | | | | | | MRPL30 |
| chr19: 48822374- | chr19: 48893109- | 1 | 1 | 1 | 0 | 0 | 1 | 1 | 0 | 0 | 0 | 5 | 0.5 | EMP3, |
| 48826115 | 48895745 | | | | | | | | | | | | | TMEM143, |
| | | | | | | | | | | | | | | SYNGR4 |
| chr10: 123810420- | chr10: 123899560- | 1 | 1 | 1 | 0 | 0 | 0 | 0 | 0 | 1 | 1 | 5 | 0.5 | TACC2 |
| 123814961 | 123903449 | | | | | | | | | | | | | |
| chr8: 33328227- | chr8: 33369546- | 1 | 1 | 0 | 1 | 0 | 1 | 1 | 0 | 0 | 0 | 5 | 0.5 | MAK16 |
| 33331667 | 33374876 | | | | | | | | | | | | | |
| chr20: 37359004- | chr20: 37378634- | 0 | 1 | 1 | 0 | 0 | 1 | 1 | 0 | 1 | 0 | 5 | 0.5 | ACTR5 |
| 37361539 | 37381544 | | | | | | | | | | | | | |
| chr2: 109208932- | chr2: 109251613- | 1 | 1 | 1 | 1 | 0 | 0 | 0 | 0 | 1 | 0 | 5 | 0.5 | LIMS1 |
| 109214709 | 109254604 | | | | | | | | | | | | | |
| chr4: 6987217- | chr4: 7071653- | 1 | 1 | 1 | 1 | 0 | 0 | 0 | 0 | 1 | 0 | 5 | 0.5 | TBC1D14, |
| 6990638 | 7073993 | | | | | | | | | | | | | CCDC96, |
| | | | | | | | | | | | | | | TADA2B, |
| | | | | | | | | | | | | | | GRPEL1 |
| chr19: 17551115- | chr19: 17576149- | 1 | 1 | 0 | 1 | 0 | 1 | 0 | 0 | 1 | 0 | 5 | 0.5 | TMEM221, |
| 17553336 | 17578849 | | | | | | | | | | | | | CTD- |
| | | | | | | | | | | | | | | 2521M24.10, |
| | | | | | | | | | | | | | | NXNL1 |
| chr14: 24582391- | chr14: 24608327- | 0 | 1 | 0 | 1 | 0 | 1 | 1 | 0 | 0 | 1 | 5 | 0.5 | RP11- |
| 24586034 | 24611892 | | | | | | | | | | | | | 468E2.6, |
| | | | | | | | | | | | | | | FITM1, |
| | | | | | | | | | | | | | | PSME1 |
| chr9: 138755027- | chr9: 138796261- | 1 | 1 | 0 | 1 | 0 | 1 | 0 | 0 | 1 | 0 | 5 | 0.5 | CAMSAP1 |
| 138758119 | 138802920 | | | | | | | | | | | | | |
| chr20: 3775328- | chr20: 3834389- | 0 | 0 | 1 | 1 | 0 | 0 | 1 | 1 | 0 | 1 | 5 | 0.5 | AP5S1, |
| 3781257 | 3837169 | | | | | | | | | | | | | MAVS |
| chr12: 6976370- | chr12: 7073285- | 0 | 1 | 1 | 1 | 0 | 1 | 1 | 0 | 0 | 0 | 5 | 0.5 | SPSB2, |
| 6979789 | 7075463 | | | | | | | | | | | | | LRRC23, |
| | | | | | | | | | | | | | | ENO2, |
| | | | | | | | | | | | | | | ATN1, |
| | | | | | | | | | | | | | | C12orf57, |
| | | | | | | | | | | | | | | PTPN6 |
| chr6: 52279938- | chr6: 52367447- | 0 | 1 | 1 | 1 | 0 | 0 | 0 | 0 | 1 | 1 | 5 | 0.5 | EFHC1 |
| 52283399 | 52371805 | | | | | | | | | | | | | |
| chr11: 112096219- | chr11: 112149146- | 0 | 1 | 1 | 1 | 0 | 1 | 1 | 0 | 0 | 0 | 5 | 0.5 | AP002884.2, |
| 112099110 | 112152508 | | | | | | | | | | | | | PLET1 |
| chr1: 9255286- | chr1: 9308967- | 0 | 1 | 0 | 0 | 1 | 1 | 1 | 0 | 1 | 0 | 5 | 0.5 | H6PD |
| 9259419 | 9313775 | | | | | | | | | | | | | |
| chr12: 31811041- | chr12: 31833801- | 1 | 1 | 0 | 1 | 0 | 0 | 1 | 1 | 0 | 0 | 5 | 0.5 | METTL20 |
| 31813842 | 31836555 | | | | | | | | | | | | | |
| chr10: 75909439- | chr10: 75971990- | 0 | 1 | 1 | 1 | 0 | 0 | 1 | 0 | 1 | 0 | 5 | 0.5 | ADK |
| 75913935 | 75975140 | | | | | | | | | | | | | |
| chr12: 48135023- | chr12: 48165828- | 1 | 1 | 0 | 1 | 0 | 1 | 0 | 1 | 0 | 0 | 5 | 0.5 | RAPGEF3, |
| 48138939 | 48168276 | | | | | | | | | | | | | SLC48A1, |
| | | | | | | | | | | | | | | RAPGEF3, |
| | | | | | | | | | | | | | | SLC48A1 |
| chr1: 154932704- | chr1: 155022035- | 0 | 1 | 1 | 0 | 0 | 1 | 0 | 0 | 1 | 1 | 5 | 0.5 | SHC1, |
| 154935672 | 155026454 | | | | | | | | | | | | | CKS1B, |
| | | | | | | | | | | | | | | FLAD1, |
| | | | | | | | | | | | | | | LENEP, |
| | | | | | | | | | | | | | | ZBTB7B, |
| | | | | | | | | | | | | | | DCST2, |
| | | | | | | | | | | | | | | DCST1 |
| chr11: 60665406- | chr11: 60680662- | 1 | 1 | 0 | 1 | 0 | 0 | 0 | 1 | 0 | 1 | 5 | 0.5 | PRPF19 |
| 60668245 | 60684363 | | | | | | | | | | | | | |
| chr1: 206911656- | chr1: 206981131- | 0 | 0 | 1 | 1 | 0 | 0 | 0 | 1 | 1 | 1 | 5 | 0.5 | IL10, |
| 206917222 | 206984268 | | | | | | | | | | | | | IL19 |
| chr10: 52126542- | chr10: 52179603- | 0 | 1 | 1 | 1 | 0 | 0 | 1 | 0 | 1 | 0 | 5 | 0.5 | AC069547.2 |
| 52128678 | 52182797 | | | | | | | | | | | | | |
| chr5: 113695532- | chr5: 113784334- | 0 | 1 | 0 | 1 | 1 | 1 | 0 | 1 | 0 | 0 | 5 | 0.5 | KCNN2 |
| 113700332 | 113787071 | | | | | | | | | | | | | |
| chr6: 40972782- | chr6: 41005822- | 0 | 0 | 0 | 1 | 0 | 0 | 1 | 1 | 1 | 1 | 5 | 0.5 | UNC5CL |
| 40975224 | 41009314 | | | | | | | | | | | | | |
| chr5: 94889048- | chr5: 94978962- | 1 | 1 | 1 | 0 | 0 | 1 | 0 | 0 | 0 | 1 | 5 | 0.5 | GPR150 |
| 94892665 | 94983614 | | | | | | | | | | | | | |
| chr19: 2018308- | chr19: 2048930- | 0 | 1 | 1 | 1 | 0 | 1 | 1 | 0 | 1 | 0 | 6 | 0.6 | MKNK2 |
| 2021074 | 2053301 | | | | | | | | | | | | | |
| chr16: 15734388- | chr16: 15797088- | 1 | 1 | 1 | 1 | 0 | 0 | 1 | 1 | 0 | 0 | 6 | 0.6 | NDE1 |
| 15739862 | 15799931 | | | | | | | | | | | | | |
| chr11: 64017662- | chr11: 64071044- | 0 | 1 | 1 | 1 | 0 | 1 | 1 | 0 | 0 | 1 | 6 | 0.6 | GPR137, |
| 64020627 | 64075138 | | | | | | | | | | | | | BAD, |
| | | | | | | | | | | | | | | GPR137, |
| | | | | | | | | | | | | | | KCNK4, |
| | | | | | | | | | | | | | | TEX40 |
| chr16: 2886714- | chr16: 2923619- | 0 | 1 | 1 | 0 | 1 | 0 | 1 | 0 | 1 | 1 | 6 | 0.6 | PRSS22 |
| 2890405 | 2926242 | | | | | | | | | | | | | |
| chr1: 154926905- | chr1: 154941001- | 1 | 1 | 1 | 0 | 0 | 1 | 1 | 1 | 0 | 0 | 6 | 0.6 | PYGO2 |
| 154929614 | 154949428 | | | | | | | | | | | | | |
| chr19: 38632152- | chr19: 38723268- | 0 | 0 | 1 | 0 | 1 | 1 | 0 | 1 | 1 | 1 | 6 | 0.6 | DPF1 |
| 38635713 | 38726259 | | | | | | | | | | | | | |
| chr3: 50472685- | chr3: 50555420- | 1 | 1 | 0 | 0 | 1 | 1 | 0 | 1 | 1 | 0 | 6 | 0.6 | CACNA2D2 |
| 50474996 | 50557881 | | | | | | | | | | | | | |
| chr14: 73370152- | chr14: 73413980- | 1 | 1 | 1 | 0 | 0 | 1 | 1 | 1 | 0 | 0 | 6 | 0.6 | DCAF4 |
| 73373904 | 73416705 | | | | | | | | | | | | | |
| chr12: 31804438- | chr12: 31833801- | 1 | 1 | 0 | 1 | 0 | 0 | 1 | 1 | 1 | 0 | 6 | 0.6 | METTL20 |
| 31807549 | 31836555 | | | | | | | | | | | | | |
| chr19: 42460954- | chr19: 42537624- | 0 | 0 | 1 | 1 | 0 | 1 | 0 | 1 | 1 | 1 | 6 | 0.6 | ATP1A3 |
| 42464743 | 42540235 | | | | | | | | | | | | | |
| chr19: 49500438- | chr19: 49575968- | 1 | 1 | 1 | 0 | 1 | 0 | 0 | 0 | 1 | 1 | 6 | 0.6 | LHB, |
| 49504098 | 49579497 | | | | | | | | | | | | | CGB, |
| | | | | | | | | | | | | | | CGB2, |
| | | | | | | | | | | | | | | CGB1, |
| | | | | | | | | | | | | | | CTB- |
| | | | | | | | | | | | | | | 60B18.6, |
| | | | | | | | | | | | | | | CGB5, |
| | | | | | | | | | | | | | | CGB1, |
| | | | | | | | | | | | | | | CGB8, |
| | | | | | | | | | | | | | | CGB7, |
| | | | | | | | | | | | | | | NTF4 |
| chr2: 232549557- | chr2: 232577905- | 0 | 1 | 1 | 1 | 1 | 0 | 1 | 0 | 1 | 0 | 6 | 0.6 | MGC4771, |
| 232553478 | 232582119 | | | | | | | | | | | | | PTMA |
| chr1: 33218634- | chr1: 33237382- | 1 | 1 | 0 | 0 | 1 | 1 | 0 | 0 | 1 | 1 | 6 | 0.6 | KIAA1522 |
| 33224103 | 33240309 | | | | | | | | | | | | | |
| chr1: 156388799- | chr1: 156413454- | 0 | 1 | 0 | 1 | 1 | 1 | 0 | 1 | 1 | 0 | 6 | 0.6 | C1orf61 |
| 156392580 | 156417052 | | | | | | | | | | | | | |
| chr1: 116247501- | chr1: 116313365- | 0 | 0 | 1 | 1 | 1 | 1 | 0 | 1 | 1 | 0 | 6 | 0.6 | CASQ2 |
| 116250851 | 116315912 | | | | | | | | | | | | | |
| chr5: 179552913- | chr5: 179634765- | 0 | 1 | 0 | 0 | 1 | 1 | 0 | 1 | 1 | 1 | 6 | 0.6 | RASGEF1C |
| 179556033 | 179637501 | | | | | | | | | | | | | |
| chr9: 140081875- | chr9: 140129343- | 1 | 0 | 0 | 1 | 0 | 1 | 1 | 0 | 1 | 1 | 6 | 0.6 | TPRN, |
| 140085966 | 140132388 | | | | | | | | | | | | | TMEM203, |
| | | | | | | | | | | | | | | NDOR1, |
| | | | | | | | | | | | | | | RNF208, |
| | | | | | | | | | | | | | | C9orf169, |
| | | | | | | | | | | | | | | RNF224, |
| | | | | | | | | | | | | | | SLC34A3 |
| chr1: 26689298- | chr1: 26742261- | 1 | 1 | 0 | 1 | 1 | 0 | 0 | 1 | 1 | 0 | 6 | 0.6 | ZNF683, |
| 26691614 | 26746384 | | | | | | | | | | | | | LIN28A |
| chr11: 82577664- | chr11: 82616161- | 0 | 1 | 1 | 1 | 0 | 1 | 0 | 1 | 1 | 0 | 6 | 0.6 | PRCP, |
| 82579923 | 82619800 | | | | | | | | | | | | | C11orf82 |
| chr3: 32380938- | chr3: 32439483- | 1 | 1 | 0 | 1 | 0 | 0 | 1 | 1 | 0 | 1 | 6 | 0.6 | CMTM7 |
| 32383527 | 32441798 | | | | | | | | | | | | | |
| chr3: 142646701- | chr3: 142704721- | 1 | 1 | 1 | 1 | 0 | 0 | 1 | 0 | 1 | 0 | 6 | 0.6 | PAQR9 |
| 142650942 | 142707763 | | | | | | | | | | | | | |
| chr16: 84309145- | chr16: 84340586- | 0 | 1 | 1 | 0 | 1 | 0 | 1 | 0 | 1 | 1 | 6 | 0.6 | WFDC1 |
| 84313389 | 84343175 | | | | | | | | | | | | | |
| chr6: 3747516- | chr6: 3823500- | 1 | 1 | 1 | 0 | 1 | 1 | 1 | 0 | 0 | 0 | 6 | 0.6 | PXDC1 |
| 3750567 | 3827466 | | | | | | | | | | | | | |
| chr2: 10121970- | chr2: 10167611- | 0 | 1 | 1 | 1 | 0 | 1 | 1 | 0 | 1 | 0 | 6 | 0.6 | GRHL1 |
| 10126417 | 10171290 | | | | | | | | | | | | | |
| chr14: 88409541- | chr14: 88478830- | 1 | 1 | 0 | 1 | 1 | 0 | 1 | 0 | 1 | 0 | 6 | 0.6 | GALC, |
| 88412979 | 88481368 | | | | | | | | | | | | | GPR65 |
| chr11: 124627652- | chr11: 124705848- | 0 | 1 | 1 | 1 | 1 | 1 | 1 | 0 | 0 | 0 | 6 | 0.6 | ESAM, |
| 124630547 | 124708804 | | | | | | | | | | | | | MSANTD2 |
| chr20: 34741660- | chr20: 34790101- | 0 | 1 | 0 | 1 | 0 | 1 | 1 | 1 | 1 | 0 | 6 | 0.6 | AL121895.1, |
| 34746206 | 34791942 | | | | | | | | | | | | | EPB41L1 |
| chr20: 52194440- | chr20: 52221841- | 0 | 1 | 1 | 1 | 0 | 0 | 0 | 1 | 1 | 1 | 6 | 0.6 | ZNF217 |
| 52198139 | 52231057 | | | | | | | | | | | | | |
| chr1: 154243247- | chr1: 154296291- | 1 | 1 | 1 | 0 | 0 | 1 | 0 | 1 | 1 | 0 | 6 | 0.6 | AQP10 |
| 154246238 | 154299979 | | | | | | | | | | | | | |
| chr13: 107187365- | chr13: 107271116- | 1 | 1 | 1 | 1 | 1 | 1 | 0 | 0 | 0 | 0 | 6 | 0.6 | ARGLU1 |
| 107190206 | 107273814 | | | | | | | | | | | | | |
| chr20: 30180548- | chr20: 30197883- | 0 | 1 | 1 | 0 | 1 | 1 | 1 | 0 | 0 | 1 | 6 | 0.6 | ID1 |
| 30184821 | 30201700 | | | | | | | | | | | | | |
| chr3: 58172165- | chr3: 58221926- | 0 | 1 | 0 | 1 | 1 | 1 | 1 | 1 | 0 | 0 | 6 | 0.6 | DNASE1L3 |
| 58175603 | 58225182 | | | | | | | | | | | | | |
| chr17: 17397909- | chr17: 17493387- | 0 | 1 | 1 | 1 | 0 | 1 | 1 | 1 | 0 | 0 | 6 | 0.6 | PEMT |
| 17401889 | 17496984 | | | | | | | | | | | | | |
| chr21: 34142314- | chr21: 34197617- | 1 | 1 | 1 | 1 | 0 | 1 | 0 | 1 | 0 | 0 | 6 | 0.6 | C21orf62 |
| 34145955 | 34200351 | | | | | | | | | | | | | |
| chr12: 54068582- | chr12: 54139455- | 0 | 0 | 1 | 0 | 1 | 1 | 1 | 1 | 1 | 0 | 6 | 0.6 | CALCOCO1 |
| 54071340 | 54141660 | | | | | | | | | | | | | |
| chr22: 36518719- | chr22: 36575247- | 0 | 1 | 1 | 1 | 1 | 0 | 1 | 0 | 1 | 0 | 6 | 0.6 | APOL3 |
| 36522329 | 36578291 | | | | | | | | | | | | | |
| chr9: 124396101- | chr9: 124445873- | 1 | 1 | 1 | 0 | 1 | 1 | 1 | 0 | 0 | 0 | 6 | 0.6 | DAB2IP |
| 124399680 | 124450524 | | | | | | | | | | | | | |
| chr17: 75445484- | chr17: 75489706- | 1 | 0 | 1 | 1 | 0 | 1 | 1 | 0 | 1 | 0 | 6 | 0.6 | 43717 |
| 75449177 | 75492589 | | | | | | | | | | | | | |
| chr17: 16288852- | chr17: 16341079- | 1 | 1 | 1 | 1 | 0 | 1 | 1 | 0 | 0 | 0 | 6 | 0.6 | TRPV2 |
| 16291777 | 16345482 | | | | | | | | | | | | | |
| chr16: 4363227- | chr16: 4451397- | 0 | 1 | 1 | 0 | 1 | 1 | 1 | 0 | 1 | 0 | 6 | 0.6 | GLIS2, |
| 4368600 | 4455411 | | | | | | | | | | | | | PAM16, |
| | | | | | | | | | | | | | | VASN |
| chr11: 128420963- | chr11: 128499046- | 0 | 1 | 1 | 1 | 1 | 1 | 0 | 1 | 0 | 0 | 6 | 0.6 | ETS1 |
| 128426748 | 128503176 | | | | | | | | | | | | | |
| chr17: 79882360- | chr17: 79977361- | 1 | 0 | 1 | 1 | 0 | 1 | 1 | 0 | 0 | 1 | 6 | 0.6 | PYCR1, |
| 79887327 | 79981279 | | | | | | | | | | | | | MYADML2, |
| | | | | | | | | | | | | | | NOTUM, |
| | | | | | | | | | | | | | | ASPSCR1 |
| chr19: 45689325- | chr19: 45750936- | 1 | 1 | 0 | 1 | 0 | 0 | 1 | 0 | 1 | 1 | 6 | 0.6 | EXOC3L2 |
| 45692220 | 45754865 | | | | | | | | | | | | | |
| chr3: 58201900- | chr3: 58283866- | 1 | 1 | 1 | 1 | 1 | 1 | 1 | 0 | 0 | 0 | 7 | 0.7 | ABHD6 |
| 58205403 | 58287230 | | | | | | | | | | | | | |
| chr1: 23879829- | chr1: 23962884- | 0 | 1 | 1 | 1 | 1 | 1 | 0 | 1 | 1 | 0 | 7 | 0.7 | ID3, |
| 23883743 | 23965746 | | | | | | | | | | | | | MDS2 |
| chr1: 111065274- | chr1: 111161057- | 0 | 1 | 0 | 1 | 1 | 1 | 0 | 1 | 1 | 1 | 7 | 0.7 | KCNA2 |
| 111068938 | 111163267 | | | | | | | | | | | | | |
| chr14: 105115566- | chr14: 105188955- | 1 | 1 | 1 | 0 | 1 | 1 | 0 | 0 | 1 | 1 | 7 | 0.7 | INF2 |
| 105119116 | 105194236 | | | | | | | | | | | | | |
| chr17: 73849191- | chr17: 73900164- | 1 | 1 | 0 | 1 | 0 | 1 | 1 | 1 | 1 | 0 | 7 | 0.7 | TRIM47, |
| 73853410 | 73902721 | | | | | | | | | | | | | TRIM65 |
| chr17: 48237661- | chr17: 48245560- | 1 | 1 | 1 | 0 | 1 | 1 | 0 | 0 | 1 | 1 | 7 | 0.7 | SGCA |
| 48240476 | 48249084 | | | | | | | | | | | | | |
| chr19: 12875945- | chr19: 12957186- | 0 | 1 | 1 | 1 | 0 | 1 | 1 | 1 | 1 | 0 | 7 | 0.7 | HOOK2, |
| 12877953 | 12959666 | | | | | | | | | | | | | JUNB, |
| | | | | | | | | | | | | | | PRDX2, |
| | | | | | | | | | | | | | | RNASEH2A, |
| | | | | | | | | | | | | | | RTBDN, |
| | | | | | | | | | | | | | | MAST1, |
| | | | | | | | | | | | | | | RTBDN, |
| | | | | | | | | | | | | | | MAST1 |
| chr14: 52509423- | chr14: 52597341- | 1 | 1 | 1 | 0 | 1 | 1 | 0 | 1 | 0 | 1 | 7 | 0.7 | NID2 |
| 52512905 | 52599988 | | | | | | | | | | | | | |
| chr17: 55974925- | chr17: 56063312- | 0 | 1 | 1 | 1 | 1 | 1 | 1 | 0 | 0 | 1 | 7 | 0.7 | CUEDC1, |
| 55985440 | 56067179 | | | | | | | | | | | | | VEZF1 |
| chr3: 183943987- | chr3: 183965860- | 0 | 1 | 1 | 1 | 1 | 1 | 0 | 1 | 0 | 1 | 7 | 0.7 | VWA5B2 |
| 183948129 | 183969202 | | | | | | | | | | | | | |
| chr11: 75038585- | chr11: 75098933- | 0 | 1 | 1 | 1 | 0 | 1 | 1 | 0 | 1 | 1 | 7 | 0.7 | ARRB1 |
| 75041505 | 75101070 | | | | | | | | | | | | | |
| chr1: 23667938- | chr1: 23728960- | 1 | 1 | 1 | 1 | 0 | 1 | 0 | 1 | 1 | 0 | 7 | 0.7 | ZNF436, |
| 23672781 | 23731702 | | | | | | | | | | | | | C1orf213, |
| | | | | | | | | | | | | | | ZNF436 |
| chr15: 64358459- | chr15: 64444370- | 1 | 1 | 0 | 1 | 0 | 1 | 0 | 1 | 1 | 1 | 7 | 0.7 | FAM96A, |
| 64362667 | 64447215 | | | | | | | | | | | | | SNX1, |
| | | | | | | | | | | | | | | SNX22 |
| chr1: 27658180- | chr1: 27720875- | 1 | 1 | 1 | 1 | 1 | 1 | 0 | 0 | 1 | 0 | 7 | 0.7 | SYTL1, |
| 27660846 | 27723091 | | | | | | | | | | | | | MAP3K6, |
| | | | | | | | | | | | | | | FCN3, |
| | | | | | | | | | | | | | | CD164L2, |
| | | | | | | | | | | | | | | GPR3 |
| chr17: 34092823- | chr17: 34130594- | 1 | 1 | 1 | 1 | 0 | 1 | 0 | 1 | 1 | 0 | 7 | 0.7 | MMP28 |
| 34095186 | 34133319 | | | | | | | | | | | | | |
| chr8: 146049422- | chr8: 146124899- | 1 | 1 | 0 | 1 | 0 | 1 | 1 | 1 | 0 | 1 | 7 | 0.7 | COMMD5 |
| 146054428 | 146128860 | | | | | | | | | | | | | |
| chr12: 56965736- | chr12: 57028301- | 1 | 1 | 1 | 1 | 0 | 0 | 0 | 1 | 1 | 1 | 7 | 0.7 | BAZ2A |
| 56968986 | 57031832 | | | | | | | | | | | | | |
| chr2: 176943287- | chr2: 176949888- | 0 | 1 | 1 | 0 | 1 | 1 | 0 | 1 | 1 | 1 | 7 | 0.7 | EVX2 |
| 176946193 | 176954131 | | | | | | | | | | | | | |
| chr9: 117248366- | chr9: 117269123- | 0 | 1 | 1 | 1 | 0 | 0 | 1 | 1 | 1 | 1 | 7 | 0.7 | DFNB31 |
| 117251402 | 117271085 | | | | | | | | | | | | | |
| chr4: 88295588- | chr4: 88342166- | 1 | 1 | 1 | 1 | 0 | 1 | 1 | 0 | 1 | 0 | 7 | 0.7 | HSD17B11 |
| 88298650 | 88345785 | | | | | | | | | | | | | |
| chr17: 6815953- | chr17: 6907388- | 1 | 1 | 1 | 1 | 0 | 1 | 0 | 1 | 1 | 0 | 7 | 0.7 | ALOX12 |
| 6819652 | 6909284 | | | | | | | | | | | | | |
| chr12: 53644304- | chr12: 53679906- | 1 | 1 | 1 | 1 | 0 | 1 | 0 | 0 | 1 | 1 | 7 | 0.7 | ESPL1 |
| 53647487 | 53684225 | | | | | | | | | | | | | |
| chr1: 145504759- | chr1: 145540925- | 1 | 1 | 1 | 0 | 0 | 1 | 1 | 0 | 1 | 1 | 7 | 0.7 | PEX11B, |
| 145509387 | 145544482 | | | | | | | | | | | | | ITGA10 |
| chr1: 54342540- | chr1: 54424197- | 1 | 1 | 1 | 1 | 0 | 1 | 1 | 0 | 1 | 0 | 7 | 0.7 | YIPF1, |
| 54345956 | 54426722 | | | | | | | | | | | | | DIO1, |
| | | | | | | | | | | | | | | HSPB11, |
| | | | | | | | | | | | | | | LRRC42 |
| chr12: 7166796- | chr12: 7260098- | 1 | 1 | 1 | 0 | 1 | 1 | 1 | 0 | 1 | 0 | 7 | 0.7 | C1R |
| 7169516 | 7263226 | | | | | | | | | | | | | |
| chr1: 160161671- | chr1: 160187931- | 1 | 1 | 1 | 1 | 1 | 1 | 1 | 0 | 0 | 0 | 7 | 0.7 | CASQ1, |
| 160164711 | 160190903 | | | | | | | | | | | | | PEA15 |
| chr19: 33614937- | chr19: 33674400- | 1 | 1 | 1 | 1 | 0 | 1 | 1 | 0 | 1 | 0 | 7 | 0.7 | WDR88 |
| 33618281 | 33676766 | | | | | | | | | | | | | |
| chr17: 46747932- | chr17: 46800569- | 1 | 1 | 0 | 1 | 1 | 1 | 1 | 0 | 1 | 0 | 7 | 0.7 | PRAC1 |
| 46750772 | 46803817 | | | | | | | | | | | | | |
| chr12: 98959017- | chr12: 99036960- | 1 | 1 | 1 | 1 | 0 | 1 | 1 | 0 | 1 | 0 | 7 | 0.7 | SLC25A3 |
| 98962276 | 99041407 | | | | | | | | | | | | | |
| chr1: 150994776- | chr1: 151070218- | 1 | 1 | 1 | 1 | 0 | 1 | 0 | 1 | 1 | 0 | 7 | 0.7 | BNIPL, |
| 150998599 | 151074703 | | | | | | | | | | | | | C1orf56, |
| | | | | | | | | | | | | | | MLLT11, |
| | | | | | | | | | | | | | | CDC42SE1, |
| | | | | | | | | | | | | | | GABPB2 |
| chr12: 121771038- | chr12: 121836117- | 1 | 1 | 1 | 1 | 0 | 1 | 1 | 1 | 0 | 0 | 7 | 0.7 | ANAPC5 |
| 121774318 | 121839760 | | | | | | | | | | | | | |
| chr21: 35880911- | chr21: 35953805- | 1 | 1 | 1 | 1 | 1 | 0 | 1 | 0 | 0 | 1 | 7 | 0.7 | KCNE1, |
| 35884566 | 35957561 | | | | | | | | | | | | | RCAN1 |
| chr2: 46218538- | chr2: 46288768- | 1 | 0 | 1 | 1 | 1 | 1 | 1 | 0 | 1 | 0 | 7 | 0.7 | PRKCE |
| 46222257 | 46291631 | | | | | | | | | | | | | |
| chr5: 175999734- | chr5: 176072836- | 1 | 1 | 1 | 1 | 0 | 1 | 0 | 0 | 1 | 1 | 7 | 0.7 | GPRIN1, |
| 176002644 | 176076409 | | | | | | | | | | | | | SNCB, |
| | | | | | | | | | | | | | | EIF4E1B |
| chr1: 210482989- | chr1: 210572842- | 1 | 0 | 1 | 1 | 1 | 1 | 1 | 1 | 0 | 0 | 7 | 0.7 | HHAT |
| 210486011 | 210575401 | | | | | | | | | | | | | |
| chr14: 60557308- | chr14: 60630361- | 1 | 1 | 1 | 1 | 0 | 1 | 1 | 0 | 0 | 1 | 7 | 0.7 | PCNXL4 |
| 60561125 | 60633596 | | | | | | | | | | | | | |
| chr11: 57478121- | chr11: 57566014- | 1 | 1 | 1 | 1 | 0 | 1 | 1 | 0 | 1 | 0 | 7 | 0.7 | C11orf31, |
| 57482722 | 57569907 | | | | | | | | | | | | | BTBD18, |
| | | | | | | | | | | | | | | CTNND1 |
| chr3: 58978667- | chr3: 59037356- | 1 | 1 | 1 | 1 | 1 | 0 | 0 | 1 | 1 | 0 | 7 | 0.7 | C3orf67 |
| 58982147 | 59039828 | | | | | | | | | | | | | |
| chr17: 80162384- | chr17: 80193213- | 1 | 1 | 1 | 1 | 0 | 0 | 1 | 0 | 1 | 1 | 7 | 0.7 | CCDC57, |
| 80165094 | 80197424 | | | | | | | | | | | | | SLC16A3 |
| chr16: 66979448- | chr16: 67010102- | 0 | 1 | 1 | 1 | 0 | 1 | 1 | 1 | 1 | 0 | 7 | 0.7 | CES3 |
| 66982341 | 67013515 | | | | | | | | | | | | | |
| chr9: 126110610- | chr9: 126146678- | 0 | 0 | 0 | 1 | 1 | 1 | 1 | 1 | 1 | 1 | 7 | 0.7 | CRB2 |
| 126113593 | 126149228 | | | | | | | | | | | | | |
| chr20: 35489484- | chr20: 35512678- | 1 | 1 | 1 | 1 | 1 | 1 | 1 | 0 | 1 | 0 | 8 | 0.8 | TLDC2 |
| 35494344 | 35517063 | | | | | | | | | | | | | |
| chr11: 118935219- | chr11: 118970279- | 0 | 1 | 1 | 1 | 0 | 1 | 1 | 1 | 1 | 1 | 8 | 0.8 | HMBS, |
| 118939489 | 118975542 | | | | | | | | | | | | | H2AFX |
| chr4: 155470878- | chr4: 155543020- | 1 | 1 | 0 | 1 | 0 | 1 | 1 | 1 | 1 | 1 | 8 | 0.8 | FGB, |
| 155474867 | 155544814 | | | | | | | | | | | | | FGA, |
| | | | | | | | | | | | | | | FGG |
| chr1: 6407114- | chr1: 6496502- | 1 | 1 | 1 | 1 | 0 | 1 | 0 | 1 | 1 | 1 | 8 | 0.8 | ACOT7, |
| 6409633 | 6499511 | | | | | | | | | | | | | HES2, |
| | | | | | | | | | | | | | | ESPN |
| chr12: 54786970- | chr12: 54831916- | 0 | 1 | 1 | 1 | 1 | 1 | 0 | 1 | 1 | 1 | 8 | 0.8 | ITGA5 |
| 54789108 | 54835009 | | | | | | | | | | | | | |
| chr9: 116842696- | chr9: 116869055- | 1 | 1 | 1 | 0 | 0 | 1 | 1 | 1 | 1 | 1 | 8 | 0.8 | KIF12 |
| 116846292 | 116872247 | | | | | | | | | | | | | |
| chr19: 12169208- | chr19: 12200511- | 0 | 1 | 1 | 1 | 1 | 1 | 1 | 1 | 1 | 0 | 8 | 0.8 | ZNF844 |
| 12171833 | 12204234 | | | | | | | | | | | | | |
| chr15: 45001794- | chr15: 45075712- | 1 | 1 | 1 | 1 | 0 | 1 | 1 | 1 | 1 | 0 | 8 | 0.8 | TRIM69 |
| 45007008 | 45078584 | | | | | | | | | | | | | |
| chr17: 8021007- | chr17: 8088809- | 1 | 1 | 0 | 0 | 1 | 1 | 1 | 1 | 1 | 1 | 8 | 0.8 | HES7, |
| 8026028 | 8094267 | | | | | | | | | | | | | PER1, |
| | | | | | | | | | | | | | | VAMP2, |
| | | | | | | | | | | | | | | RP11- |
| | | | | | | | | | | | | | | 599B13.6, |
| | | | | | | | | | | | | | | VAMP2, |
| | | | | | | | | | | | | | | TMEM107 |
| chr19: 49976370- | chr19: 50072437- | 0 | 1 | 1 | 1 | 0 | 1 | 1 | 1 | 1 | 1 | 8 | 0.8 | RPL13A, |
| 49980428 | 50075548 | | | | | | | | | | | | | RPS11, |
| | | | | | | | | | | | | | | hsa-mir-150, |
| | | | | | | | | | | | | | | FCGRT, |
| | | | | | | | | | | | | | | RCN3 |
| chr5: 148695817- | chr5: 148745317- | 1 | 1 | 1 | 1 | 0 | 1 | 1 | 0 | 1 | 1 | 8 | 0.8 | GRPEL2, |
| 148698262 | 148748184 | | | | | | | | | | | | | PCYOX1L |
| chr16: 85088230- | chr16: 85118946- | 0 | 1 | 1 | 1 | 1 | 1 | 1 | 0 | 1 | 1 | 8 | 0.8 | KIAA0513 |
| 85092434 | 85121932 | | | | | | | | | | | | | |
| chr19: 39541813- | chr19: 39599455- | 1 | 1 | 1 | 1 | 1 | 1 | 0 | 0 | 1 | 1 | 8 | 0.8 | PAPL |
| 39545225 | 39603821 | | | | | | | | | | | | | |
| chr20: 62585685- | chr20: 62603306- | 0 | 1 | 0 | 1 | 1 | 1 | 1 | 1 | 1 | 1 | 8 | 0.8 | ZNF512B |
| 62589597 | 62606942 | | | | | | | | | | | | | |
| chr17: 72652291- | chr17: 72739517- | 1 | 1 | 0 | 1 | 1 | 1 | 1 | 0 | 1 | 1 | 8 | 0.8 | RAB37, |
| 72655112 | 72742241 | | | | | | | | | | | | | CD300LF, |
| | | | | | | | | | | | | | | RAB37 |
| chr5: 171613376- | chr5: 171707549- | 1 | 1 | 1 | 1 | 0 | 1 | 1 | 1 | 0 | 1 | 8 | 0.8 | EFCAB9 |
| 171617275 | 171712451 | | | | | | | | | | | | | |
| chr20: 37273631- | chr20: 37359004- | 0 | 1 | 1 | 1 | 1 | 1 | 1 | 1 | 1 | 0 | 8 | 0.8 | SLC32A1 |
| 37276165 | 37361539 | | | | | | | | | | | | | |
| chr1: 21834204- | chr1: 21920568- | 1 | 1 | 1 | 1 | 1 | 1 | 0 | 1 | 1 | 0 | 8 | 0.8 | ALPL |
| 21839294 | 21923668 | | | | | | | | | | | | | |
| chr14: 24800597- | chr14: 24880264- | 0 | 1 | 1 | 1 | 0 | 1 | 1 | 1 | 1 | 1 | 8 | 0.8 | RP11- |
| 24804390 | 24882632 | | | | | | | | | | | | | 934B9.3, |
| | | | | | | | | | | | | | | RIPK3, |
| | | | | | | | | | | | | | | NFATC4, |
| | | | | | | | | | | | | | | NYNRIN |
| chr10: 102777760- | chr10: 102825442- | 1 | 1 | 1 | 1 | 1 | 1 | 1 | 0 | 1 | 0 | 8 | 0.8 | PDZD7, |
| 102779751 | 102829077 | | | | | | | | | | | | | SFXN3, |
| | | | | | | | | | | | | | | KAZALD1 |
| chr1: 156807533- | chr1: 156895440- | 1 | 1 | 1 | 0 | 1 | 0 | 1 | 1 | 1 | 1 | 8 | 0.8 | INSRR, |
| 156810316 | 156898664 | | | | | | | | | | | | | NTRK1, |
| | | | | | | | | | | | | | | PEAR1, |
| | | | | | | | | | | | | | | LRRC71 |
| chr20: 5092441- | chr20: 5171695- | 1 | 1 | 1 | 1 | 0 | 1 | 1 | 1 | 1 | 0 | 8 | 0.8 | PCNA, |
| 5095273 | 5174574 | | | | | | | | | | | | | CDS2 |
| chr12: 48201078- | chr12: 48229990- | 1 | 1 | 1 | 1 | 1 | 1 | 0 | 1 | 1 | 0 | 8 | 0.8 | HDAC7 |
| 48208432 | 48233708 | | | | | | | | | | | | | |
| chr11: 62160011- | chr11: 62190499- | 1 | 1 | 1 | 0 | 1 | 1 | 0 | 1 | 1 | 1 | 8 | 0.8 | SCGB1A1 |
| 62165002 | 62195083 | | | | | | | | | | | | | |
| chr3: 50296330- | chr3: 50360795- | 1 | 1 | 1 | 0 | 1 | 1 | 1 | 0 | 1 | 1 | 8 | 0.8 | LSMEM2, |
| 50298790 | 50363340 | | | | | | | | | | | | | IFRD2, |
| | | | | | | | | | | | | | | HYAL3, |
| | | | | | | | | | | | | | | NAT6, |
| | | | | | | | | | | | | | | HYAL3, |
| | | | | | | | | | | | | | | NAT6, |
| | | | | | | | | | | | | | | HYAL3, |
| | | | | | | | | | | | | | | NAT6, |
| | | | | | | | | | | | | | | HYAL3, |
| | | | | | | | | | | | | | | HYAL1, |
| | | | | | | | | | | | | | | HYAL2 |
| chr17: 74347135- | chr17: 74379209- | 1 | 1 | 1 | 1 | 0 | 1 | 1 | 0 | 1 | 1 | 8 | 0.8 | SPHK1 |
| 74351990 | 74383436 | | | | | | | | | | | | | |
| chr6: 157734022- | chr6: 157800136- | 1 | 1 | 1 | 0 | 1 | 1 | 1 | 1 | 1 | 0 | 8 | 0.8 | TMEM242 |
| 157737404 | 157805158 | | | | | | | | | | | | | |
| chr1: 159129573- | chr1: 159165945- | 1 | 1 | 1 | 1 | 1 | 1 | 0 | 1 | 1 | 0 | 8 | 0.8 | CADM3 |
| 159132207 | 159169204 | | | | | | | | | | | | | |
| chr11: 66344893- | chr11: 66382833- | 1 | 1 | 1 | 1 | 0 | 1 | 1 | 0 | 1 | 1 | 8 | 0.8 | CCS, |
| 66348080 | 66387525 | | | | | | | | | | | | | CCDC87, |
| | | | | | | | | | | | | | | CCS |
| chr11: 57223913- | chr11: 57258300- | 0 | 1 | 1 | 1 | 1 | 1 | 1 | 1 | 1 | 0 | 8 | 0.8 | RTN4RL2 |
| 57228015 | 57262331 | | | | | | | | | | | | | |
| chr16: 4249316- | chr16: 4302626- | 1 | 1 | 1 | 1 | 0 | 1 | 1 | 0 | 1 | 1 | 8 | 0.8 | SRL |
| 4251639 | 4305128 | | | | | | | | | | | | | |
| chr1: 156552043- | chr1: 156569143- | 0 | 1 | 1 | 1 | 1 | 1 | 1 | 1 | 1 | 0 | 8 | 0.8 | APOA1BP |
| 156555542 | 156573503 | | | | | | | | | | | | | |
| chr2: 238329157- | chr2: 238382811- | 1 | 1 | 1 | 1 | 1 | 1 | 0 | 1 | 0 | 1 | 8 | 0.8 | AC112721.1 |
| 238332016 | 238385558 | | | | | | | | | | | | | |
| chr19: 56115485- | chr19: 56141971- | 0 | 1 | 1 | 1 | 1 | 1 | 1 | 1 | 0 | 1 | 8 | 0.8 | ZNF784 |
| 56119148 | 56145190 | | | | | | | | | | | | | |
| chr7: 99678043- | chr7: 99727755- | 1 | 1 | 0 | 1 | 1 | 1 | 0 | 1 | 1 | 1 | 8 | 0.8 | COPS6, |
| 99681158 | 99731577 | | | | | | | | | | | | | MCM7, |
| | | | | | | | | | | | | | | AP4M1, |
| | | | | | | | | | | | | | | MCM7, |
| | | | | | | | | | | | | | | AP4M1, |
| | | | | | | | | | | | | | | TAF6, |
| | | | | | | | | | | | | | | CNPY4, |
| | | | | | | | | | | | | | | TAF6, |
| | | | | | | | | | | | | | | MBLAC1 |
| chr1: 159100982- | chr1: 159180406- | 1 | 1 | 1 | 1 | 1 | 1 | 0 | 1 | 1 | 0 | 8 | 0.8 | CADM3, |
| 159103474 | 159185139 | | | | | | | | | | | | | DARC |
| chr6: 90020015- | chr6: 90077099- | 1 | 1 | 1 | 0 | 1 | 1 | 1 | 0 | 1 | 1 | 8 | 0.8 | GABRR2, |
| 90023546 | 90080340 | | | | | | | | | | | | | UBE2J1 |
| chr10: 102752781- | chr10: 102770766- | 1 | 1 | 1 | 0 | 1 | 1 | 1 | 0 | 1 | 1 | 8 | 0.8 | LZTS2 |
| 102755343 | 102775402 | | | | | | | | | | | | | |
| chr19: 58837387- | chr19: 58917608- | 0 | 1 | 0 | 1 | 1 | 1 | 1 | 1 | 1 | 1 | 8 | 0.8 | A1BG, |
| 58841634 | 58921705 | | | | | | | | | | | | | ZNF497, |
| | | | | | | | | | | | | | | ZNF837, |
| | | | | | | | | | | | | | | RPS5, |
| | | | | | | | | | | | | | | AC012313.1 |
| chr17: 39893137- | chr17: 39966533- | 1 | 1 | 1 | 1 | 1 | 1 | 1 | 0 | 0 | 1 | 8 | 0.8 | JUP |
| 39895797 | 39971004 | | | | | | | | | | | | | |
| chr20: 30538256- | chr20: 30618132- | 1 | 1 | 1 | 0 | 1 | 1 | 1 | 0 | 1 | 1 | 8 | 0.8 | XKR7, |
| 30541299 | 30620865 | | | | | | | | | | | | | CCM2L |
| chr17: 1623097- | chr17: 1686059- | 1 | 1 | 1 | 1 | 0 | 1 | 1 | 0 | 1 | 1 | 8 | 0.8 | WDR81, |
| 1627088 | 1688612 | | | | | | | | | | | | | SERPINF2, |
| | | | | | | | | | | | | | | SERPINF1 |
| chr7: 134230942- | chr7: 134289800- | 1 | 1 | 1 | 1 | 0 | 1 | 1 | 0 | 1 | 1 | 8 | 0.8 | AKR1B15 |
| 134234600 | 134292567 | | | | | | | | | | | | | |
| chr17: 37361862- | chr17: 37399754- | 1 | 1 | 1 | 1 | 1 | 1 | 1 | 1 | 1 | 0 | 9 | 0.9 | STAC2 |
| 37365935 | 37403272 | | | | | | | | | | | | | |
| chr11: 62552839- | chr11: 62628238- | 1 | 1 | 1 | 1 | 1 | 1 | 1 | 1 | 1 | 0 | 9 | 0.9 | TMEM223, |
| 62556009 | 62631659 | | | | | | | | | | | | | NXF1, |
| | | | | | | | | | | | | | | STX5, |
| | | | | | | | | | | | | | | WDR74, |
| | | | | | | | | | | | | | | SLC3A2 |
| chr10: 4855995- | chr10: 4890292- | 1 | 1 | 1 | 0 | 1 | 1 | 1 | 1 | 1 | 1 | 9 | 0.9 | AKR1E2 |
| 4859770 | 4893553 | | | | | | | | | | | | | |
| chr1: 181052643- | chr1: 181134487- | 0 | 1 | 1 | 1 | 1 | 1 | 1 | 1 | 1 | 1 | 9 | 0.9 | IER5 |
| 181054950 | 181137029 | | | | | | | | | | | | | |
| chr22: 19615615- | chr22: 19704344- | 1 | 1 | 1 | 0 | 1 | 1 | 1 | 1 | 1 | 1 | 9 | 0.9 | 43713 |
| 19620711 | 19706935 | | | | | | | | | | | | | |
| chr17: 73669168- | chr17: 73743031- | 1 | 1 | 1 | 1 | 0 | 1 | 1 | 1 | 1 | 1 | 9 | 0.9 | ITGB4 |
| 73672984 | 73747096 | | | | | | | | | | | | | |
| chr7: 128857338- | chr7: 128910210- | 1 | 1 | 1 | 1 | 1 | 0 | 1 | 1 | 1 | 1 | 9 | 0.9 | AHCYL2 |
| 128860161 | 128913092 | | | | | | | | | | | | | |
| chr19: 10378759- | chr19: 10442356- | 0 | 1 | 1 | 1 | 1 | 1 | 1 | 1 | 1 | 1 | 9 | 0.9 | ICAM4, |
| 10382537 | 10447358 | | | | | | | | | | | | | ICAM5, |
| | | | | | | | | | | | | | | ZGLP1, |
| | | | | | | | | | | | | | | FDX1L |
| chr1: 45264192- | chr1: 45284174- | 1 | 1 | 1 | 1 | 1 | 1 | 1 | 1 | 1 | 0 | 9 | 0.9 | TCTEX1D4, |
| 45267271 | 45287472 | | | | | | | | | | | | | BTBD19 |
| chr22: 38053460- | chr22: 38076199- | 1 | 1 | 1 | 1 | 1 | 1 | 1 | 1 | 1 | 0 | 9 | 0.9 | PDXP, |
| 38058354 | 38078722 | | | | | | | | | | | | | LGALS1 |
| chr16: 71914274- | chr16: 71993937- | 1 | 1 | 1 | 1 | 0 | 1 | 1 | 1 | 1 | 1 | 9 | 0.9 | IST1 |
| 71919164 | 71996919 | | | | | | | | | | | | | |
| chr13: 76054235- | chr13: 76122278- | 1 | 1 | 1 | 1 | 1 | 1 | 1 | 1 | 1 | 0 | 9 | 0.9 | COMMD6 |
| 76057719 | 76125224 | | | | | | | | | | | | | |
| chr17: 37224411- | chr17: 37307982- | 1 | 1 | 1 | 1 | 1 | 1 | 1 | 1 | 1 | 0 | 9 | 0.9 | PLXDC1 |
| 37227414 | 37311418 | | | | | | | | | | | | | |
| chr15: 89599801- | chr15: 89671343- | 1 | 1 | 1 | 1 | 1 | 0 | 1 | 1 | 1 | 1 | 9 | 0.9 | ABHD2 |
| 89602453 | 89674295 | | | | | | | | | | | | | |
| chr16: 2886714- | chr16: 2975986- | 1 | 1 | 1 | 0 | 1 | 1 | 1 | 1 | 1 | 1 | 9 | 0.9 | PRSS22, |
| 2890405 | 2978389 | | | | | | | | | | | | | FLYWCH2, |
| | | | | | | | | | | | | | | FLYWCH1 |
| chr7: 128037491- | chr7: 128098314- | 1 | 1 | 1 | 1 | 1 | 1 | 1 | 0 | 1 | 1 | 9 | 0.9 | IMPDH1, |
| 128039971 | 128102626 | | | | | | | | | | | | | HILPDA |
| chr11: 118269113- | chr11: 118358432- | 1 | 1 | 1 | 1 | 0 | 1 | 1 | 1 | 1 | 1 | 9 | 0.9 | RP11- |
| 118274250 | 118361138 | | | | | | | | | | | | | 770J1.4, |
| | | | | | | | | | | | | | | KMT2A |
| chr16: 67188495- | chr16: 67216893- | 1 | 1 | 1 | 1 | 1 | 1 | 1 | 0 | 1 | 1 | 9 | 0.9 | FBXL8, |
| 67191296 | 67221169 | | | | | | | | | | | | | TRADD, |
| | | | | | | | | | | | | | | HSF4, |
| | | | | | | | | | | | | | | NOL3 |
| chr3: 134026137- | chr3: 134096278- | 1 | 1 | 0 | 1 | 1 | 1 | 1 | 1 | 1 | 1 | 9 | 0.9 | AMOTL2 |
| 134029616 | 134098433 | | | | | | | | | | | | | |
| chr1: 208056912- | chr1: 208135510- | 1 | 1 | 1 | 1 | 1 | 1 | 1 | 1 | 1 | 0 | 9 | 0.9 | CD34 |
| 208060409 | 208138581 | | | | | | | | | | | | | |
| chr17: 74403479- | chr17: 74453798- | 1 | 1 | 1 | 1 | 1 | 1 | 1 | 0 | 1 | 1 | 9 | 0.9 | UBE2O, |
| 74406384 | 74457580 | | | | | | | | | | | | | AANAT |
| chr14: 71087772- | chr14: 71179466- | 1 | 1 | 1 | 1 | 1 | 1 | 1 | 1 | 0 | 1 | 9 | 0.9 | TTC9 |
| 71090297 | 71182024 | | | | | | | | | | | | | |
| chr19: 46270026- | chr19: 46301580- | 1 | 1 | 1 | 1 | 0 | 1 | 1 | 1 | 1 | 1 | 9 | 0.9 | DMPK, |
| 46275014 | 46304038 | | | | | | | | | | | | | DMWD |
| chr9: 36247010- | chr9: 36327313- | 1 | 1 | 1 | 1 | 0 | 1 | 1 | 1 | 1 | 1 | 9 | 0.9 | GNE |
| 36250829 | 36330633 | | | | | | | | | | | | | |
| chr11: 8934815- | chr11: 8979976- | 1 | 1 | 1 | 1 | 1 | 1 | 1 | 1 | 1 | 0 | 9 | 0.9 | C11orf16, |
| 8937367 | 8982355 | | | | | | | | | | | | | ASCL3 |
| chr11: 66600408- | chr11: 66647903- | 1 | 1 | 1 | 1 | 1 | 1 | 0 | 1 | 1 | 1 | 9 | 0.9 | RCE1, |
| 66603785 | 66654040 | | | | | | | | | | | | | PC, |
| | | | | | | | | | | | | | | LRFN4 |
| chr5: 176828776- | chr5: 176851808- | 1 | 1 | 1 | 1 | 0 | 1 | 1 | 1 | 1 | 1 | 9 | 0.9 | F12 |
| 176832820 | 176857046 | | | | | | | | | | | | | |
| chr1: 156109729- | chr1: 156193573- | 1 | 1 | 1 | 1 | 1 | 1 | 1 | 1 | 1 | 0 | 9 | 0.9 | SEMA4A, |
| 156112262 | 156197013 | | | | | | | | | | | | | SLC25A44, |
| | | | | | | | | | | | | | | PMF1- |
| | | | | | | | | | | | | | | BGLAP, |
| | | | | | | | | | | | | | | PMF1, |
| | | | | | | | | | | | | | | PMF1- |
| | | | | | | | | | | | | | | BGLAP, |
| | | | | | | | | | | | | | | PMF1, |
| | | | | | | | | | | | | | | PMF1- |
| | | | | | | | | | | | | | | BGLAP |
| chr2: 220322188- | chr2: 220393289- | 0 | 1 | 1 | 1 | 1 | 1 | 1 | 1 | 1 | 1 | 9 | 0.9 | GMPPA, |
| 220326146 | 220396306 | | | | | | | | | | | | | ASIC4 |
| chr11: 128753720- | chr11: 128824993- | 1 | 1 | 1 | 1 | 1 | 1 | 1 | 1 | 1 | 0 | 9 | 0.9 | KCNJ5, |
| 128757230 | 128828080 | | | | | | | | | | | | | C11orf45, |
| | | | | | | | | | | | | | | KCNJ5, |
| | | | | | | | | | | | | | | TP53AIP1 |
| chr7: 44894587- | chr7: 44960611- | 1 | 1 | 1 | 1 | 0 | 1 | 1 | 1 | 1 | 1 | 9 | 0.9 | PURB |
| 44898621 | 44962790 | | | | | | | | | | | | | |
| chr19: 18387804- | chr19: 18438455- | 1 | 1 | 1 | 1 | 1 | 1 | 1 | 1 | 1 | 0 | 9 | 0.9 | LSM4 |
| 18394224 | 18440850 | | | | | | | | | | | | | |
| chr15: 75131983- | chr15: 75192845- | 1 | 1 | 1 | 1 | 0 | 1 | 1 | 1 | 1 | 1 | 9 | 0.9 | SCAMP2, |
| 75138665 | 75195488 | | | | | | | | | | | | | MPI |
| chr17: 46800569- | chr17: 46867155- | 1 | 1 | 1 | 1 | 1 | 1 | 1 | 0 | 1 | 1 | 9 | 0.9 | HOXB13 |
| 46803817 | 46869924 | | | | | | | | | | | | | |
| chr7: 100079812- | chr7: 100156362- | 1 | 1 | 1 | 1 | 1 | 1 | 1 | 0 | 1 | 1 | 9 | 0.9 | NYAP1, |
| 100082989 | 100158790 | | | | | | | | | | | | | AGFG2 |
| chr16: 68561282- | chr16: 68622986- | 1 | 1 | 1 | 1 | 1 | 1 | 1 | 0 | 1 | 1 | 9 | 0.9 | ZFP90 |
| 68564601 | 68627379 | | | | | | | | | | | | | |
| chr1: 201415872- | chr1: 201480610- | 1 | 1 | 1 | 1 | 0 | 1 | 1 | 1 | 1 | 1 | 9 | 0.9 | PHLDA3, |
| 201418962 | 201483741 | | | | | | | | | | | | | CSRP1 |
| chr10: 111965368- | chr10: 112037708- | 1 | 1 | 1 | 1 | 1 | 1 | 1 | 1 | 1 | 0 | 9 | 0.9 | MXI1 |
| 111971747 | 112040496 | | | | | | | | | | | | | |
| chr8: 67024245- | chr8: 67087874- | 1 | 1 | 1 | 1 | 1 | 1 | 1 | 0 | 1 | 1 | 9 | 0.9 | TRIM55 |
| 67028006 | 67091088 | | | | | | | | | | | | | |
| chr20: 30147113- | chr20: 30197883- | 0 | 1 | 1 | 1 | 1 | 1 | 1 | 1 | 1 | 1 | 9 | 0.9 | ID1 |
| 30149407 | 30201700 | | | | | | | | | | | | | |
| chr10: 54499478- | chr10: 54537908- | 1 | 1 | 1 | 1 | 1 | 1 | 1 | 1 | 0 | 1 | 9 | 0.9 | MBL2 |
| 54503284 | 54541189 | | | | | | | | | | | | | |
| chr17: 34090226- | chr17: 34130594- | 1 | 1 | 1 | 1 | 1 | 1 | 1 | 1 | 1 | 0 | 9 | 0.9 | MMP28 |
| 34092546 | 34133319 | | | | | | | | | | | | | |
| chr3: 184088258- | chr3: 184133427- | 1 | 1 | 1 | 0 | 1 | 1 | 1 | 1 | 1 | 1 | 9 | 0.9 | THPO, |
| 184090957 | 184137285 | | | | | | | | | | | | | CHRD, |
| | | | | | | | | | | | | | | RP11- |
| | | | | | | | | | | | | | | 433C9.2 |
| chr19: 8417389- | chr19: 8458841- | 1 | 1 | 1 | 1 | 1 | 1 | 1 | 1 | 1 | 0 | 9 | 0.9 | ANGPTL4, |
| 8422977 | 8463787 | | | | | | | | | | | | | RAB11B |
| chr14: 24482313- | chr14: 24526405- | 1 | 1 | 1 | 1 | 0 | 1 | 1 | 1 | 1 | 1 | 9 | 0.9 | LRRC16B |
| 24484756 | 24529130 | | | | | | | | | | | | | |
| chr2: 113893615- | chr2: 113960681- | 1 | 1 | 1 | 1 | 1 | 0 | 1 | 1 | 1 | 1 | 9 | 0.9 | PSD4 |
| 113897419 | 113963045 | | | | | | | | | | | | | |
| chr11: 94799400- | chr11: 94883497- | 1 | 1 | 1 | 1 | 1 | 1 | 1 | 1 | 1 | 0 | 9 | 0.9 | ENDOD1 |
| 94804422 | 94888833 | | | | | | | | | | | | | |
| chr10: 102100521- | chr10: 102192388- | 1 | 1 | 1 | 1 | 1 | 1 | 1 | 0 | 1 | 1 | 9 | 0.9 | SCD |
| 102103165 | 102194881 | | | | | | | | | | | | | |
| chr1: 21580314- | chr1: 21659825- | 1 | 1 | 1 | 1 | 1 | 1 | 1 | 1 | 1 | 0 | 9 | 0.9 | ECE1 |
| 21583273 | 21663403 | | | | | | | | | | | | | |
| chr17: 37885487- | chr17: 37909241- | 1 | 1 | 1 | 1 | 1 | 1 | 1 | 1 | 1 | 0 | 9 | 0.9 | GRB7 |
| 37887988 | 37913141 | | | | | | | | | | | | | |
| chr19: 42375263- | chr19: 42437732- | 0 | 1 | 1 | 1 | 1 | 1 | 1 | 1 | 1 | 1 | 9 | 0.9 | CD79A, |
| 42378720 | 42441372 | | | | | | | | | | | | | ARHGEF1 |
| chr9: 112230738- | chr9: 112281336- | 1 | 1 | 1 | 1 | 1 | 1 | 1 | 1 | 0 | 1 | 9 | 0.9 | PTPN3 |
| 112233547 | 112284427 | | | | | | | | | | | | | |
| chr15: 90440918- | chr15: 90513627- | 0 | 1 | 1 | 1 | 1 | 1 | 1 | 1 | 1 | 1 | 9 | 0.9 | C15orf38, |
| 90443333 | 90516253 | | | | | | | | | | | | | C15orf38- |
| | | | | | | | | | | | | | | AP3S2, |
| | | | | | | | | | | | | | | C15orf38 |
| chr17: 36578605- | chr17: 36598443- | 1 | 1 | 1 | 1 | 1 | 1 | 1 | 1 | 1 | 0 | 9 | 0.9 | ARHGAP23 |
| 36581525 | 36601623 | | | | | | | | | | | | | |
| chr17: 56427946- | chr17: 56492280- | 1 | 1 | 0 | 1 | 1 | 1 | 1 | 1 | 1 | 1 | 9 | 0.9 | RNF43 |
| 56431498 | 56496219 | | | | | | | | | | | | | |
| chr1: 206662306- | chr1: 206717020- | 1 | 1 | 1 | 1 | 1 | 1 | 1 | 0 | 1 | 1 | 9 | 0.9 | C1orf147, |
| 206665067 | 206719618 | | | | | | | | | | | | | RASSF5 |
| chr19: 3072847- | chr19: 3161896- | 1 | 1 | 1 | 1 | 1 | 1 | 1 | 1 | 1 | 0 | 9 | 0.9 | GNA11, |
| 3077398 | 3164772 | | | | | | | | | | | | | GNA15 |
| chr20: 62610605- | chr20: 62687833- | 1 | 1 | 1 | 1 | 0 | 1 | 1 | 1 | 1 | 1 | 9 | 0.9 | ZNF512B, |
| 62613415 | 62690863 | | | | | | | | | | | | | SOX18 |
| chr8: 101858355- | chr8: 101949297- | 1 | 1 | 1 | 1 | 0 | 1 | 1 | 1 | 1 | 1 | 9 | 0.9 | YWHAZ |
| 101861163 | 101952346 | | | | | | | | | | | | | |
| chr2: 219716911- | chr2: 219761037- | 1 | 1 | 1 | 1 | 1 | 1 | 1 | 1 | 1 | 0 | 9 | 0.9 | WNT6, |
| 219719710 | 219764524 | | | | | | | | | | | | | WNT10A |
| chr19: 11907082- | chr19: 11990339- | 1 | 1 | 1 | 1 | 1 | 1 | 1 | 1 | 1 | 0 | 9 | 0.9 | ZNF440, |
| 11910542 | 11993819 | | | | | | | | | | | | | ZNF439 |
| chr5: 176828776- | chr5: 176871126- | 0 | 1 | 1 | 1 | 1 | 1 | 1 | 1 | 1 | 1 | 9 | 0.9 | F12, |
| 176832820 | 176876017 | | | | | | | | | | | | | GRK6 |
| chr1: 159767115- | chr1: 159859391- | 1 | 1 | 1 | 1 | 1 | 1 | 0 | 1 | 1 | 1 | 9 | 0.9 | FCRL6, |
| 159770807 | 159863577 | | | | | | | | | | | | | SLAMF8, |
| | | | | | | | | | | | | | | C1orf204, |
| | | | | | | | | | | | | | | VSIG8 |
| chr17: 48558744- | chr17: 48623329- | 1 | 1 | 1 | 1 | 1 | 1 | 1 | 0 | 1 | 1 | 9 | 0.9 | MYCBPAP, |
| 48560985 | 48625599 | | | | | | | | | | | | | EPN3 |
| chr20: 48503368- | chr20: 48543945- | 1 | 1 | 1 | 1 | 0 | 1 | 1 | 1 | 1 | 1 | 9 | 0.9 | SPATA2 |
| 48507341 | 48546640 | | | | | | | | | | | | | |
| chr1: 154170510- | chr1: 154243247- | 1 | 1 | 1 | 1 | 1 | 1 | 1 | 1 | 1 | 1 | 10 | 1 | C1orf189, |
| 154173159 | 154246238 | | | | | | | | | | | | | UBAP2L, |
| | | | | | | | | | | | | | | C1orf43, |
| | | | | | | | | | | | | | | UBAP2L |
| chr11: 61100111- | chr11: 61151524- | 1 | 1 | 1 | 1 | 1 | 1 | 1 | 1 | 1 | 1 | 10 | 1 | CYB561A3, |
| 61105271 | 61154529 | | | | | | | | | | | | | TMEM138, |
| | | | | | | | | | | | | | | CYB561A3, |
| | | | | | | | | | | | | | | TMEM138 |
| chr17: 4468185- | chr17: 4527726- | 1 | 1 | 1 | 1 | 1 | 1 | 1 | 1 | 1 | 1 | 10 | 1 | SMTNL2 |
| 4471414 | 4531006 | | | | | | | | | | | | | |
| chr9: 133740938- | chr9: 133813950- | 1 | 1 | 1 | 1 | 1 | 1 | 1 | 1 | 1 | 1 | 10 | 1 | QRFP, |
| 133742945 | 133818796 | | | | | | | | | | | | | FIBCD1 |
| chr20: 4087646- | chr20: 4151445- | 1 | 1 | 1 | 1 | 1 | 1 | 1 | 1 | 1 | 1 | 10 | 1 | SMOX |
| 4090992 | 4155046 | | | | | | | | | | | | | |
| chr22: 25346582- | chr22: 25443512- | 1 | 1 | 1 | 1 | 1 | 1 | 1 | 1 | 1 | 1 | 10 | 1 | KIAA1671 |
| 25350027 | 25446238 | | | | | | | | | | | | | |
| chr12: 57399306- | chr12: 57453458- | 1 | 1 | 1 | 1 | 1 | 1 | 1 | 1 | 1 | 1 | 10 | 1 | TAC3, |
| 57403571 | 57458541 | | | | | | | | | | | | | MYO1A |
| chr14: 24526405- | chr14: 24576721- | 1 | 1 | 1 | 1 | 1 | 1 | 1 | 1 | 1 | 1 | 10 | 1 | CPNE6, |
| 24529130 | 24579288 | | | | | | | | | | | | | NRL, |
| | | | | | | | | | | | | | | PCK2, |
| | | | | | | | | | | | | | | NRL |
| chr11: 64876705- | chr11: 64947063- | 1 | 1 | 1 | 1 | 1 | 1 | 1 | 1 | 1 | 1 | 10 | 1 | ZNHIT2, |
| 64881308 | 64950910 | | | | | | | | | | | | | FAU, |
| | | | | | | | | | | | | | | MRPL49, |
| | | | | | | | | | | | | | | FAU, |
| | | | | | | | | | | | | | | SYVN1, |
| | | | | | | | | | | | | | | SPDYC |
| chr10: 104592519- | chr10: 104675500- | 1 | 1 | 1 | 1 | 1 | 1 | 1 | 1 | 1 | 1 | 10 | 1 | CYP17A1, |
| 104596445 | 104679322 | | | | | | | | | | | | | C10orf32, |
| | | | | | | | | | | | | | | AS3MT |
| chr20: 17917381- | chr20: 17975448- | 1 | 1 | 1 | 1 | 1 | 1 | 1 | 1 | 1 | 1 | 10 | 1 | SNX5, |
| 17921500 | 17978619 | | | | | | | | | | | | | MGME1, |
| | | | | | | | | | | | | | | SNX5, |
| | | | | | | | | | | | | | | MGME1 |
| chr10: 99550570- | chr10: 99627812- | 1 | 1 | 1 | 1 | 1 | 1 | 1 | 1 | 1 | 1 | 10 | 1 | GOLGA7B |
| 99553435 | 99630529 | | | | | | | | | | | | | |
| chr9: 129701128- | chr9: 129726822- | 1 | 1 | 1 | 1 | 1 | 1 | 1 | 1 | 1 | 1 | 10 | 1 | RALGPS1 |
| 129704660 | 129729436 | | | | | | | | | | | | | |
| chr22: 31484346- | chr22: 31543966- | 1 | 1 | 1 | 1 | 1 | 1 | 1 | 1 | 1 | 1 | 10 | 1 | SMTN, |
| 31486270 | 31547439 | | | | | | | | | | | | | SELM, |
| | | | | | | | | | | | | | | INPP5J, |
| | | | | | | | | | | | | | | PLA2G3 |
| chr10: 102818698- | chr10: 102825442- | 1 | 1 | 1 | 1 | 1 | 1 | 1 | 1 | 1 | 1 | 10 | 1 | KAZALD1 |
| 102821386 | 102829077 | | | | | | | | | | | | | |
| chr5: 177501148- | chr5: 177589919- | 1 | 1 | 1 | 1 | 1 | 1 | 1 | 1 | 1 | 1 | 10 | 1 | N4BP3, |
| 177506053 | 177592643 | | | | | | | | | | | | | RMND5B, |
| | | | | | | | | | | | | | | NHP2 |
| chr9: 35789143- | chr9: 35811198- | 1 | 1 | 1 | 1 | 1 | 1 | 1 | 1 | 1 | 1 | 10 | 1 | NPR2 |
| 35791801 | 35816366 | | | | | | | | | | | | | |
| chr17: 72739517- | chr17: 72763895- | 1 | 1 | 1 | 1 | 1 | 1 | 1 | 1 | 1 | 1 | 10 | 1 | SLC9A3R1 |
| 72742241 | 72768141 | | | | | | | | | | | | | |
| chr19: 49296105- | chr19: 49344614- | 1 | 1 | 1 | 1 | 1 | 1 | 1 | 1 | 1 | 1 | 10 | 1 | BCAT2, |
| 49299748 | 49348541 | | | | | | | | | | | | | HSD17B14 |
| chr17: 48472847- | chr17: 48555181- | 1 | 1 | 1 | 1 | 1 | 1 | 1 | 1 | 1 | 1 | 10 | 1 | ACSF2, |
| 48476636 | 48557902 | | | | | | | | | | | | | CHAD |
| chr15: 73928038- | chr15: 73991SIS- | 1 | 1 | 1 | 1 | 1 | 1 | 1 | 1 | 1 | 1 | 10 | 1 | CD276 |
| 73930686 | 73993692 | | | | | | | | | | | | | |
| chr17: 73317601- | chr17: 73397631- | 1 | 1 | 1 | 1 | 1 | 1 | 1 | 1 | 1 | 1 | 10 | 1 | GRB2 |
| 73320845 | 73403003 | | | | | | | | | | | | | |
| chr15: 65022543- | chr15: 65100582- | 1 | 1 | 1 | 1 | 1 | 1 | 1 | 1 | 1 | 1 | 10 | 1 | RBPMS2 |
| 65025610 | 65103999 | | | | | | | | | | | | | |
| chr6: 29704750- | chr6: 29801150- | 1 | 1 | 1 | 1 | 1 | 1 | 1 | 1 | 1 | 1 | 10 | 1 | HLA-G |
| 29707709 | 29804264 | | | | | | | | | | | | | |
| chr6: 37450912- | chr6: 37532698- | 1 | 1 | 1 | 1 | 1 | 1 | 1 | 1 | 1 | 1 | 10 | 1 | CCDC167 |
| 37453968 | 37535891 | | | | | | | | | | | | | |
| chr1: 40070752- | chr1: 40117951- | 1 | 1 | 1 | 1 | 1 | 1 | 1 | 1 | 1 | 1 | 10 | 1 | HEYL |
| 40073279 | 40120705 | | | | | | | | | | | | | |
| chr1: 155219247- | chr1: 155292333- | 1 | 1 | 1 | 1 | 1 | 1 | 1 | 1 | 1 | 1 | 10 | 1 | FAM189B, |
| 155221921 | 155296036 | | | | | | | | | | | | | SCAMP3, |
| | | | | | | | | | | | | | | CLK2, |
| | | | | | | | | | | | | | | HCN3, |
| | | | | | | | | | | | | | | CLK2, |
| | | | | | | | | | | | | | | PKLR, |
| | | | | | | | | | | | | | | FDPS, |
| | | | | | | | | | | | | | | RUSC1 |
| chr2: 68977676- | chr2: 69063501- | 1 | 1 | 1 | 1 | 1 | 1 | 1 | 1 | 1 | 1 | 10 | 1 | ARHGAP25 |
| 68980347 | 69067687 | | | | | | | | | | | | | |
| chr11: 65132905- | chr11: 65182705- | 1 | 1 | 1 | 1 | 1 | 1 | 1 | 1 | 1 | 1 | 10 | 1 | SLC25A45, |
| 65135385 | 65185564 | | | | | | | | | | | | | FRMD8 |
| chr8: 142393954- | chr8: 142440154- | 1 | 1 | 1 | 1 | 1 | 1 | 1 | 1 | 1 | 1 | 10 | 1 | PTP4A3 |
| 142399131 | 142443151 | | | | | | | | | | | | | |
| chr19: 42611432- | chr19: 42687362- | 1 | 1 | 1 | 1 | 1 | 1 | 1 | 1 | 1 | 1 | 10 | 1 | POU2F2 |
| 42614252 | 42690416 | | | | | | | | | | | | | |
| chr17: 43224615- | chr17: 43273714- | 1 | 1 | 1 | 1 | 1 | 1 | 1 | 1 | 1 | 1 | 10 | 1 | HEXIM2 |
| 43230163 | 43276612 | | | | | | | | | | | | | |
| chr19: 39033970- | chr19: 39124240- | 1 | 1 | 1 | 1 | 1 | 1 | 1 | 1 | 1 | 1 | 10 | 1 | MAP4K1, |
| 39036738 | 39128589 | | | | | | | | | | | | | EIF3K |
| chr1: 154970263- | chr1: 154988501- | 1 | 1 | 1 | 1 | 1 | 1 | 1 | 1 | 1 | 1 | 10 | 1 | ZBTB7B |
| 154976774 | 154991335 | | | | | | | | | | | | | |
| chr22: 18223415- | chr22: 18311663- | 1 | 1 | 1 | 1 | 1 | 1 | 1 | 1 | 1 | 1 | 10 | 1 | BID |
| 18227047 | 18315058 | | | | | | | | | | | | | |
| chr11: 61212529- | chr11: 61283172- | 1 | 1 | 1 | 1 | 1 | 1 | 1 | 1 | 1 | 1 | 10 | 1 | PPP1R32, |
| 61215362 | 61286087 | | | | | | | | | | | | | LRRC10B |
| chr12: 120793003- | chr12: 120866768- | 1 | 1 | 1 | 1 | 1 | 1 | 1 | 1 | 1 | 1 | 10 | 1 | MSI1 |
| 120797181 | 120869789 | | | | | | | | | | | | | |
| chr5: 137784235- | chr5: 137838339- | 1 | 1 | 1 | 1 | 1 | 1 | 1 | 1 | 1 | 1 | 10 | 1 | EGR1 |
| 137786807 | 137841774 | | | | | | | | | | | | | |
| chr1: 117278707- | chr1: 117358635- | 1 | 1 | 1 | 1 | 1 | 1 | 1 | 1 | 1 | 1 | 10 | 1 | CD2 |
| 117282232 | 117361905 | | | | | | | | | | | | | |
| chr19: 13087454- | chr19: 13170550- | 1 | 1 | 1 | 1 | 1 | 1 | 1 | 1 | 1 | 1 | 10 | 1 | NFIX |
| 13090398 | 13173708 | | | | | | | | | | | | | |
| chr1: 918175- | chr1: 997526- | 1 | 1 | 1 | 1 | 1 | 1 | 1 | 1 | 1 | 1 | 10 | 1 | HES4, |
| 921513 | 1001452 | | | | | | | | | | | | | ISG15, |
| | | | | | | | | | | | | | | AGRN |
| chr8: 86131647- | chr8: 86200631- | 1 | 1 | 1 | 1 | 1 | 1 | 1 | 1 | 1 | 1 | 10 | 1 | RP11- |
| 86134438 | 86203597 | | | | | | | | | | | | | 219B4.5, |
| | | | | | | | | | | | | | | CA13, |
| | | | | | | | | | | | | | | RP11- |
| | | | | | | | | | | | | | | 219B4.6 |
| chr1: 27882316- | chr1: 27931638- | 1 | 1 | 1 | 1 | 1 | 1 | 1 | 1 | 1 | 1 | 10 | 1 | AHDC1 |
| 27885229 | 27936673 | | | | | | | | | | | | | |
| chr19: 17969511- | chr19: 18041936- | 1 | 1 | 1 | 1 | 1 | 1 | 1 | 1 | 1 | 1 | 10 | 1 | RPL18A, |
| 17971269 | 18044789 | | | | | | | | | | | | | SLC5A5 |
| chr12: 53605090- | chr12: 53660733- | 1 | 1 | 1 | 1 | 1 | 1 | 1 | 1 | 1 | 1 | 10 | 1 | RARG, |
| 53608891 | 53663583 | | | | | | | | | | | | | MFSD5 |
| chr19: 44248270- | chr19: 44287940- | 1 | 1 | 1 | 1 | 1 | 1 | 1 | 1 | 1 | 1 | 10 | 1 | SMG9, |
| 44251114 | 44290953 | | | | | | | | | | | | | KCNN4 |
| chr12: 49145765- | chr12: 49188139- | 1 | 1 | 1 | 1 | 1 | 1 | 1 | 1 | 1 | 1 | 10 | 1 | ADCY6 |
| 49148723 | 49191452 | | | | | | | | | | | | | |
| chr2: 238419935- | chr2: 238479371- | 1 | 1 | 1 | 1 | 1 | 1 | 1 | 1 | 1 | 1 | 10 | 1 | PRLH |
| 238423011 | 238482287 | | | | | | | | | | | | | |
| chr11: 65546300- | chr11: 65584385- | 1 | 1 | 1 | 1 | 1 | 1 | 1 | 1 | 1 | 1 | 10 | 1 | OVOL1 |
| 65549622 | 65587031 | | | | | | | | | | | | | |
| chr20: 48735571- | chr20: 48784626- | 1 | 1 | 1 | 1 | 1 | 1 | 1 | 1 | 1 | 1 | 10 | 1 | TMEM189, |
| 48739163 | 48787002 | | | | | | | | | | | | | TMEM189- |
| | | | | | | | | | | | | | | UBE2V1, |
| | | | | | | | | | | | | | | TMEM189 |
| chr10: 75607562- | chr10: 75699045- | 1 | 1 | 1 | 1 | 1 | 1 | 1 | 1 | 1 | 1 | 10 | 1 | CAMK2G, |
| 75611459 | 75701912 | | | | | | | | | | | | | PLAU, |
| | | | | | | | | | | | | | | C10orf55 |
| chr17: 74298030- | chr17: 74392651- | 1 | 1 | 1 | 1 | 1 | 1 | 1 | 1 | 1 | 1 | 10 | 1 | QRICH2, |
| 74301276 | 74395483 | | | | | | | | | | | | | PRPSAP1, |
| | | | | | | | | | | | | | | SPHK1 |
| chr12: 69683626- | chr12: 69750190- | 1 | 1 | 1 | 1 | 1 | 1 | 1 | 1 | 1 | 1 | 10 | 1 | LYZ |
| 69686220 | 69754673 | | | | | | | | | | | | | |
| chr20: 32378880- | chr20: 32449769- | 1 | 1 | 1 | 1 | 1 | 1 | 1 | 1 | 1 | 1 | 10 | 1 | CHMP4B |
| 32382044 | 32452627 | | | | | | | | | | | | | |
| chr2: 219164966- | chr2: 219259743- | 1 | 1 | 1 | 1 | 1 | 1 | 1 | 1 | 1 | 1 | 10 | 1 | PNKD, |
| 219167759 | 219263463 | | | | | | | | | | | | | C2orf62, |
| | | | | | | | | | | | | | | SLC11A1 |
| chr17: 40113924- | chr17: 40175796- | 1 | 1 | 1 | 1 | 1 | 1 | 1 | 1 | 1 | 1 | 10 | 1 | CNP, |
| 40116199 | 40178348 | | | | | | | | | | | | | NKIRAS2, |
| | | | | | | | | | | | | | | DNAJC7, |
| | | | | | | | | | | | | | | NKIRAS2 |
| chr19: 48215639- | chr19: 48290905- | 1 | 1 | 1 | 1 | 1 | 1 | 1 | 1 | 1 | 1 | 10 | 1 | GLTSCR2, |
| 48218352 | 48294640 | | | | | | | | | | | | | SEPW1 |
| chr5: 149836191- | chr5: 149921972- | 1 | 1 | 1 | 1 | 1 | 1 | 1 | 1 | 1 | 1 | 10 | 1 | NDST1 |
| 149841661 | 149925963 | | | | | | | | | | | | | |
| chr11: 64613697- | chr11: 64652561- | 1 | 1 | 1 | 1 | 1 | 1 | 1 | 1 | 1 | 1 | 10 | 1 | EHD1 |
| 64617783 | 64657379 | | | | | | | | | | | | | |
| chr17: 39734692- | chr17: 39803128- | 1 | 1 | 1 | 1 | 1 | 1 | 1 | 1 | 1 | 1 | 10 | 1 | KRT14, |
| 39737515 | 39805811 | | | | | | | | | | | | | KRT16, |
| | | | | | | | | | | | | | | KRT17 |
| chr15: 79199434- | chr15: 79268896- | 1 | 1 | 1 | 1 | 1 | 1 | 1 | 1 | 1 | 1 | 10 | 1 | CTSH |
| 79203968 | 79273405 | | | | | | | | | | | | | |
| chr9: 35825075- | chr9: 35880598- | 1 | 1 | 1 | 1 | 1 | 1 | 1 | 1 | 1 | 1 | 10 | 1 | FAM221B, |
| 35828120 | 35884075 | | | | | | | | | | | | | TMEM8B, |
| | | | | | | | | | | | | | | OR13J1 |
| chr7: 99509204- | chr7: 99587458- | 1 | 1 | 1 | 1 | 1 | 1 | 1 | 1 | 1 | 1 | 10 | 1 | TRIM4, |
| 99512426 | 99589835 | | | | | | | | | | | | | GJC3, |
| | | | | | | | | | | | | | | AZGP1 |
| chr11: 75513056- | chr11: 75577667- | 1 | 1 | 1 | 1 | 1 | 1 | 1 | 1 | 1 | 1 | 10 | 1 | UVRAG |
| 75515951 | 75580813 | | | | | | | | | | | | | |
| chr6: 30069162- | chr6: 30136572- | 1 | 1 | 1 | 1 | 1 | 1 | 1 | 1 | 1 | 1 | 10 | 1 | TRIM31, |
| 30072405 | 30140179 | | | | | | | | | | | | | TRIM40, |
| | | | | | | | | | | | | | | TRIM10, |
| | | | | | | | | | | | | | | TRIM15 |
| chr19: 19476739- | chr19: 19515325- | 1 | 1 | 1 | 1 | 1 | 1 | 1 | 1 | 1 | 1 | 10 | 1 | GATAD2A |
| 19479696 | 19519113 | | | | | | | | | | | | | |
| chr8: 142093035- | chr8: 142169714- | 1 | 1 | 1 | 1 | 1 | 1 | 1 | 1 | 1 | 1 | 10 | 1 | DENND3 |
| 142097500 | 142172813 | | | | | | | | | | | | | |
| chr11: 112089439- | chr11: 112149146- | 1 | 1 | 1 | 1 | 1 | 1 | 1 | 1 | 1 | 1 | 10 | 1 | PTS, |
| 112092480 | 112152508 | | | | | | | | | | | | | AP002884.2, |
| | | | | | | | | | | | | | | PLET1 |
| chr1: 3368327- | chr1: 3398581- | 1 | 1 | 1 | 1 | 1 | 1 | 1 | 1 | 1 | 1 | 10 | 1 | ARHGEF16 |
| 3371594 | 3401761 | | | | | | | | | | | | | |
| chr1: 156065260- | chr1: 156098391- | 1 | 1 | 1 | 1 | 1 | 1 | 1 | 1 | 1 | 1 | 10 | 1 | LMNA |
| 156067863 | 156101335 | | | | | | | | | | | | | |
| chr5: 176815993- | chr5: 176828776- | 1 | 1 | 1 | 1 | 1 | 1 | 1 | 1 | 1 | 1 | 10 | 1 | PFN3 |
| 176819246 | 176832820 | | | | | | | | | | | | | |
| chr19: 30099737- | chr19: 30181005- | 1 | 1 | 1 | 1 | 1 | 1 | 1 | 1 | 1 | 1 | 10 | 1 | PLEKHF1 |
| 30103252 | 30183031 | | | | | | | | | | | | | |
| chr19: 47259189- | chr19: 47342300- | 1 | 1 | 1 | 1 | 1 | 1 | 1 | 1 | 1 | 1 | 10 | 1 | SLC1A5 |
| 47262059 | 47345730 | | | | | | | | | | | | | |
| chr1: 200938586- | chr1: 201010957- | 1 | 1 | 1 | 1 | 1 | 1 | 1 | 1 | 1 | 1 | 10 | 1 | KIF21B |
| 200942434 | 201013995 | | | | | | | | | | | | | |
| chr14: 77175342- | chr14: 77249905- | 1 | 1 | 1 | 1 | 1 | 1 | 1 | 1 | 1 | 1 | 10 | 1 | VASH1 |
| 77178167 | 77252314 | | | | | | | | | | | | | |

Specificity index can also be calculated using alternative experimental data, for example 4C data which does not require a specific pull-down step. Data from a 4C-seq experiment from multiple cell lines or treatment conditions will be processed using the 4Cseqpipe processing pipeline, which outputs a list of significant loops. Then the specificity index will be calculated as described in Formula 1 above.

### Example 2: Calculating Integrity Index (IntInd)

Formula 2 below describes how prevalent a loop is within a population of a single type of cells. For instance, a loop that is present in every cell in the population will have an IntInd of 1. A loop that “breathes” and is present in about half of the cells in the population at any given time will have an IntIndᵢ of about 0.5. A loop that permanently closed in about half of the cells and permanently open in the other half of the cells would also have an IntInd of about 0.5. A loop that is never present in this cell type will have an IntInd of 0. In some situations, it is advantageous to disrupt a loop that has a high integrity index (e.g., of 0.5-1), which has a strong effect on transcription in a large number of cells in the population. In some situations, it is advantageous to disrupt a loop that has a moderate integrity index (e.g., of about 0.25-0.75), because this loop may be more susceptible to disruption than a high integrity index loop, due to “breathing” making binding sites accessible to a disrupting agent.

Formula 2:

$${IntInd}_{i} = min ( \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} , 1 )$$

The frequency of a loop can be measured, e.g., by an experimental technique such as ChIA-PET, HiChIP, HiC, or 4C-seq.

IntInd Calculation Using ChIA-PET in Gm12878

In this example, A CTCF ChIA-PET dataset (Tang et al. CTCF-Mediated Human 3D Genome Architecture Reveals Chromatin Topology for Transcription (2015). Cell 163(7):1611-27.) was used to compute IntInd for CTCF mediated loops in Gm12878 cells. The ChIA-PET data was processed using a custom pipeline based on the ChIA-PET2 software as described in Li et al. ChIA-PET2: a versatile and flexible pipeline for ChIA-PET data analysis (2017). Nucleic Acids Research 45(1):e4. Briefly, the pipeline consists of the following steps:
1. Alignment was performed as described in step 1 of the pipeline in Example 1 above.
2. Making a BEDPE file with unique paired end tags (PETs) was performed as described in step 2 of the pipeline in Example 1 above.
3. Peak calling was performed as described in step 3 of the pipeline in Example 1 above.
4. PET clustering/loop calling was performed as described in step 4 of the pipeline in Example 1 above.
5. Loop significance calling and filtering:
   a. Loop significance was calculated using the MICC2.R script provided as part of the ChIA-PET2 software. This command uses a slightly modified version of the MICC algorithm (6) to examine the files from step 4b and compute a p-value and FDR q-value for a loop call between each pair of peaks.
   b. A custom R script was used to filter the MICC output to include only peaks with FDR qvalue less than 0.05. This filtered list of loops was used for the integrity index calculation using Formula 3 described below.
      The integrity index (IntInd) for a loop i was calculated according to Formula 3:

$${IntInd}_{i} = min ( \frac{\begin{matrix}log 2 ( number of PETs supporting \\ genomic complex ( e . g . , ASMC ) i )\end{matrix}}{Normalization factor} , 1 )$$

where the normalization factor is the 99^{th} percentile of the base-2 logarithm of the number of PETs supporting any single loop. Under Formula 3, the most abundant loop measured in a cell sample has an integrity index of 1, a loop that is not detected in the cell sample will have an integrity index of 0, and a loop that “breathes” or is stably present in only a subset of cells will have an intermediate integrity index. In some situations, it is advantageous to disrupt a loop that has a high integrity index (e.g., of 0.5-1), in order to strongly affect transcription in a large number of cells in the population. In some situations, it is advantageous to disrupt a loop that has a moderate integrity index (e.g., of about 0.25-0.75), because this loop may be more susceptible to disruption than a high integrity index loop, due to “breathing” making binding sites accessible to a disrupting agent.

Formula 3 is similar to formula 2 above, but uses the base-2 logarithm of the number of PETs supporting the loop, and uses a normalization factor that is the 99^{th} percentile of the base-2 logarithm of the number of PETs supporting any single loop.

TABLE 5
Some representative loops with their associated IntInd values.

| Left | Right | Number of | log(Number | | |
|---|---|---|---|---|---|
| anchor | anchor | PETs | of PETs) | IntInd | GeneList |
| chr12: 116672859- | chr12: 116713330- | 1 | 0.000 | 0.00 | MED13L |
| 116675209 | 116716369 | | | | |
| chr15: 91428179- | chr15: 91444033- | 1 | 0.000 | 0.00 | MAN2A2 |
| 91430402 | 91446510 | | | | |
| chr16: 67480261- | chr16: 67553686- | 1 | 0.000 | 0.00 | ATP6V0D1, AGRP |
| 67482960 | 67556459 | | | | |
| chr17: 40554432- | chr17: 40578808- | 1 | 0.000 | 0.00 | PTRF |
| 40556489 | 40582411 | | | | |
| chr1: 23865188- | chr1: 23915635- | 2 | 0.301 | 0.20 | ID3 |
| 23867340 | 23919574 | | | | |
| chr9: 129975941- | chr9: 130072371- | 2 | 0.301 | 0.20 | GARNL3 |
| 129978080 | 130074566 | | | | |
| chr14: 51239723- | chr14: 51325906- | 3 | 0.477 | 0.31 | NIN |
| 51241273 | 51328408 | | | | |
| chr10: 71980460- | chr10: 72031412- | 4 | 0.602 | 0.39 | PPA1 |
| 71982873 | 72033752 | | | | |
| chr16: 3135351- | chr16: 3143681- | 4 | 0.602 | 0.39 | ZSCAN10 |
| 3138452 | 3146860 | | | | |
| chr22: 37867170- | chr22: 37893169- | 5 | 0.699 | 0.46 | MFNG |
| 37870006 | 37895334 | | | | |
| chr11: 33708455- | chr11: 33762642- | 6 | 0.778 | 0.51 | C11orf91, CD59 |
| 33710502 | 33765307 | | | | |
| chr1: 51394023- | chr1: 51432459- | 7 | 0.845 | 0.55 | FAF1, CDKN2C |
| 51396733 | 51435068 | | | | |
| chr1: 171612506- | chr1: 171684113- | 7 | 0.845 | 0.55 | MYOC |
| 171616510 | 171686160 | | | | |
| chr1: 161149385- | chr1: 161194624- | 8 | 0.903 | 0.59 | ADAMTS4, |
| 161151597 | 161198401 | | | | NDUFS2, FCER1G, |
| | | | | | AL590714.1, |
| | | | | | APOA2, TOMM40L |
| chr22: 19704565- | chr22: 19717229- | 8 | 0.903 | 0.59 | GP1BB |
| 19706734 | 19720349 | | | | |
| chr1: 33831748- | chr1: 33920292- | 9 | 0.954 | 0.62 | PHC2 |
| 33834506 | 33922466 | | | | |
| chr12: 54972781- | chr12: 54995899- | 9 | 0.954 | 0.62 | PPP1R1A |
| 54975166 | 54998132 | | | | |
| chr17: 46150285- | chr17: 46207302- | 9 | 0.954 | 0.62 | CBX1, SNX11 |
| 46153258 | 46209908 | | | | |
| chr19: 12875886- | chr19: 12948004- | 9 | 0.954 | 0.62 | HOOK2, JUNB, |
| 12878306 | 12950492 | | | | PRDX2, |
| | | | | | RNASEH2A, |
| | | | | | RTBDN, MAST1, |
| | | | | | RTBDN, MAST1 |
| chr19: 10981264- | chr19: 11046654- | 11 | 1.041 | 0.68 | YIPF2, C19orf52 |
| 10984207 | 11049205 | | | | |
| chrX: 51077659- | chrX: 51162300- | 12 | 1.079 | 0.70 | CXorf67 |
| 51080946 | 51164988 | | | | |
| chr8: 21909532- | chr8: 21993832- | 13 | 1.114 | 0.73 | DMTN, FAM160B2, |
| 21913652 | 21996234 | | | | NUDT18, HR |
| chr17: 38457306- | chr17: 38472842- | 15 | 1.176 | 0.77 | RARA |
| 38461767 | 38475708 | | | | |
| chr16: 30545725- | chr16: 30637669- | 16 | 1.204 | 0.79 | ZNF764, |
| 30548321 | 30641577 | | | | AC002310.13, |
| | | | | | ZNF764, ZNF688, |
| | | | | | ZNF785, ZNF689 |
| chr11: 64553840- | chr11: 64611244- | 17 | 1.230 | 0.80 | MAP4K2, MEN1, |
| 64556066 | 64616706 | | | | CDC42BPG |
| chr16: 75232205- | chr16: 75266326- | 18 | 1.255 | 0.82 | CTRB2, CTRB1 |
| 75234946 | 75269360 | | | | |
| chr1: 110087732- | chr1: 110171637- | 22 | 1.342 | 0.88 | GNAI3, GNAT2, |
| 110090109 | 110174324 | | | | AMPD2 |
| chr2: 113893758- | chr2: 113930562- | 27 | 1.431 | 0.93 | PSD4 |
| 113897109 | 113933882 | | | | |
| chr5: 16488700- | chr5: 16549420- | 46 | 1.663 | 1.00 | FAM134B |
| 16490685 | 16553823 | | | | |
| chr2: 11832322- | chr2: 11918217- | 53 | 1.724 | 1.00 | LPIN1 |
| 11834325 | 11920619 | | | | |
| chr1: 19985727- | chr1: 20029446- | 67 | 1.826 | 1.00 | HTR6 |
| 19989839 | 20032917 | | | | |
| chr17: 3639837- | chr17: 3729207- | 93 | 1.968 | 1.00 | ITGAE |
| 3642439 | 3731975 | | | | |

IntInd Calculation Using HiChIP in Hepa1.6

Integrity index may also be calculated using data from a HiChIP experiment, as described herein. HiChIP (Mumbach et al. HiChIP: efficient and sensitive analysis of protein-directed genome architecture (2016). Nature Methods. 13(11):919-922) data will be generated for CTCF in Hepa1.6 cells. The data will be processed using the HiC-Pro (Servant et al. HiC-Pro: an optimized and flexible pipeline for Hi-C data processing (2015). Genome Biology 16:259) software, which generates PETs from the raw sequencing reads. In parallel, CTCF ChIP-seq data will be generated from Hepa1.6 cells, aligned using bowtie2 (Langmead et al. Fast gapped-read alignment with Bowtie 2 (2012). Nature Methods 9:357-359), duplicates removed using the Picard MarkDuplicates command (Broad Institute. Picard (2019), https://broadinstitute.github.io/picard/), and peaks called using MACS2 (https://github.com/taoliu/MACS). These PETs will then be provided to the hichipper software package (Lareau et al. hichipper: a preprocessing pipeline for calling DNA loops from HiChIP data (2018). Nature Methods 15(3):155-156) to associate PETs with peak pairs and to assign a significance value to these peak pairs (loops) using the Mango algorithm (Phanstiel et al. Mango: a bias-correcting ChIA-PET analysis pipeline (2015). Bioinformatics 31(19):3092-8.). Loops with an FDR q-value less than 0.05 will be retained. Formula 3 will be used to compute integrity indices.

### Example 3: Calculating the Integrity Index of Selected Genes

Integrity index was calculated for MYC, FOXJ3, TUSC5, DAND5, TTC21B, SHMT2, CDK6 in the Gm12878 cell line data, using the method described in Example 2. The results are shown in Table 6.

TABLE 6
Integrity index of selected genes.

| gene | chr1 | start1 | end1 | chr2 | start2 | end2 | cAB |
|---|---|---|---|---|---|---|---|
| MYC | chr8 | 128745952 | 128746887 | chr8 | 129663970 | 129668069 | 5 |
| MYC | chr8 | 128746090 | 128746745 | chr8 | 129375379 | 129379288 | 2 |
| MYC | chr8 | 127777265 | 127777866 | chr8 | 128745911 | 128746538 | 6 |
| MYC | chr8 | 128227119 | 128228132 | chr8 | 128745777 | 128746734 | 4 |
| MYC | chr8 | 128737786 | 128738529 | chr8 | 128745864 | 128746871 | 4 |
| FOXJ3 | chr1 | 42612335 | 42613220 | chr1 | 42638588 | 42639955 | 11 |
| FOXJ3 | chr1 | 41953773 | 41962710 | chr1 | 42637401 | 42641758 | 9 |
| TUSC5 | chr17 | 1177053 | 1182268 | chr17 | 1233979 | 1238190 | 54 |
| TUSC5 | chr17 | 1160730 | 1164657 | chr17 | 1234249 | 1237928 | 23 |
| DAND5 | chr19 | 13075452 | 13076663 | chr19 | 13093967 | 13095090 | 6 |
| DAND5 | chr19 | 13076058 | 13076747 | chr19 | 13134286 | 13136279 | 3 |
| TTC21B | chr2 | 166810322 | 166811369 | chr2 | 166826897 | 166827886 | 5 |
| SHMT2 | chr12 | 57607983 | 57608630 | chr12 | 57623787 | 57625290 | 5 |
| CDK6 | chr7 | 92138826 | 92142437 | chr7 | 92684747 | 92685556 | 3 |

| | | | −LOG1 | | | |
|---|---|---|---|---|---|---|
| | | | 0(1 − | | | |
| gene | cA | cB | PostProb) | fdr | logpetcount | ii |
| MYC | 65 | 47 | 5.363457479 | 0 | 0.69897 | 0.35461197 |
| MYC | 57 | 17 | 2.188327879 | 0.00115046 | 0.30103 | 0.15272306 |
| MYC | 6 | 65 | 0.48416059 | 0.04388702 | 0.77815125 | 0.39478338 |
| MYC | 19 | 74 | 2.944221178 | 2.23E−04 | 0.60205999 | 0.30544612 |
| MYC | 16 | 74 | 2.530003685 | 5.16E−04 | 0.60205999 | 0.30544612 |
| FOXJ3 | 64 | 95 | 6.679786043 | 0 | 1.04139269 | 0.52833498 |
| FOXJ3 | 67 | 127 | 6.713619917 | 0 | 0.95424251 | 0.48412065 |
| TUSC5 | 210 | 170 | 10.1928844 | 0 | 1.73239376 | 0.87890403 |
| TUSC5 | 101 | 168 | 8.363393781 | 0 | 1.36172784 | 0.69085223 |
| DAND5 | 272 | 21 | 3.471057927 | 1.08E−04 | 0.77815125 | 0.39478338 |
| DAND5 | 197 | 19 | 2.719551306 | 3.05E−04 | 0.47712125 | 0.24206032 |
| TTC21B | 83 | 19 | 3.092967287 | 1.82E−04 | 0.69897 | 0.35461197 |
| SHMT2 | 338 | 14 | 2.317830269 | 9.06E−04 | 0.69897 | 0.35461197 |
| CDK6 | 10 | 164 | 1.14389778 | 0.00909646 | 0.47712125 | 0.24206032 |

1. A method of disrupting a genomic complex, e.g., anchor sequence mediated conjunction (ASMC), in a mammalian subject, comprising:
administering to a subject a disrupting agent targeted to the genomic complex (e.g., ASMC),
wherein the genomic complex (e.g., ASMC) has, or is identified as having, an IntInch, measured by Formula 2
$$( {IntInd}_{i} = min ( \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} , 1 ) ) ,$$
of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0).

2. A method of disrupting a genomic complex, e.g., anchor sequence mediated conjunction (ASMC), in a mammalian subject, comprising:
administering to a subject a disrupting agent targeted to the genomic complex (e.g., ASMC),
wherein the genomic complex (e.g., ASMC) is present in a target cell type, and
wherein the genomic complex (e.g., ASMC) is present in less than 9, 8, 7, 6, 5, 4, 3, 2, or 1 reference cell types of Table 2.

3. The method of claim 2, wherein the target cell type of is chosen from: neuronal cells, myocytes (e.g., cardiomyocytes), immune cells, endothelial cells, hepatocytes, CD34+ cells, CD3+ cells, and fibroblasts.

4. A disrupting agent that specifically binds a genomic complex (e.g., anchor sequence-mediated conjunction (ASMC)),
wherein the genomic complex (e.g., ASMC) has, or is identified as having, an IntInch, measured by Formula 2
$$( {IntInd}_{i} = min ( \frac{\begin{matrix}Frequency of genomic complex \\ ( e . g . , ASMC ) i in cell sample\end{matrix}}{\begin{matrix}95 th percentile frequency of all genomic \\ complexes ( e . g . , ASMCs ) within cell sample\end{matrix}} , 1 ) ) ,$$
of between 0.25-0.75 (e.g., 0.3-0.4, 0.4-0.5, 0.5-0.6, or 0.6-0.7), or of between 0.5-1 (e.g., about 0.5-0.6, 0.6-0.7, 0.7-0.8, 0.8-0.9, or 0.9-1.0).

5. A disrupting agent that specifically binds a genomic complex (e.g., anchor sequence-mediated conjunction (ASMC)),
wherein the genomic complex (e.g., ASMC) is present in a target cell type, and
wherein the genomic complex (e.g., ASMC) is present in less than 9, 8, 7, 6, 5, 4, 3, 2, or 1 reference cell types of Table 2.

6. The disrupting agent of either of claim 4 or 5, wherein the disrupting agent comprises a nucleic acid complementary to DNA sequence of the genomic complex (e.g., ASMC).

7. The method or composition of any of claim 1 or 4, wherein the IntIndᵢ is measured using ChIA-PET, e.g., against CTCF, e.g., as described in Example 2.

8. The method of any of claim 2 or 5 wherein genomic complex (e.g., ASMC) presence is measured by ChIA-PET, e.g., against cohesin, e.g., using an assay of Example 1.

9. The method of any of claim 1 or 4, wherein the cell sample is a cell line sample or a primary cell sample (e.g., a biopsy sample).

10. The method of any of the preceding claims, wherein the disrupting agent comprises a DNA-binding moiety that binds specifically to one or more target anchor sequences within a cell and not to non-targeted anchor sequences within the cell with sufficient affinity that it competes with binding of an endogenous nucleating polypeptide within the cell.

11. The method of any of the preceding claims, wherein the disrupting agent comprises (i) a site-specific targeting moiety and (ii) a deaminating agent.

12. The method of any of the preceding claims, wherein the disrupting agent comprises (i) a fusion polypeptide comprising an enzymatically inactive Cas polypeptide and a deaminating agent, or a nucleic acid encoding the fusion polypeptide; and (ii) a guide RNA, wherein the guide RNA targets the fusion polypeptide to an anchor sequence comprised by the ASMC.

13. The method of any of the preceding claims, wherein the disrupting agent comprises (i) a site-specific targeting moiety and (ii) an epigenetic modifying agent, e.g., wherein the epigenetic modifying agent is selected from a DNA methylase, DNA demethylase, histone methyltransferase, a histone deacetylase, or any combination thereof.

14. The method of any of the preceding claims, wherein the disrupting agent comprises (i) a fusion polypeptide comprising an enzymatically inactive Cas polypeptide and an epigenetic modifying agent, or a nucleic acid encoding the fusion polypeptide; and (ii) a guide RNA, wherein the guide RNA targets the fusion polypeptide to an anchor sequence comprised by the genomic complex (e.g., ASMC).

15. The method of any of the preceding embodiments, wherein the disrupting agent comprises a fusion polypeptide comprising a TAL effector molecule and an epigenetic modifying agent, or a nucleic acid encoding the fusion polypeptide, wherein the TAL effector molecule targets the fusion polypeptide to an anchor sequence comprised by the genomic complex (e.g., ASMC).

16. The method of any of the preceding embodiments, wherein the disrupting agent comprises a fusion polypeptide comprising a Zn finger molecule and an epigenetic modifying agent, or a nucleic acid encoding the fusion polypeptide, wherein the Zn finger molecule targets the fusion polypeptide to an anchor sequence comprised by the genomic complex (e.g., ASMC).

17. The method of any of the preceding claims, wherein the IntIndᵢ as measured by Formula 2 in a cell of the subject, is reduced to less than 0.3-0.4, 0.4-0.5, 0.5-0.6, 0.7-0.8, or 0.8-0.9.

18. The method of any of the preceding claims, wherein the IntIndᵢ as measured by Formula 2 in a cell of the subject, is reduced by at least 0.1, 0.2, 0.3, 0.4, 0.5, 0.6, 0.7. 0.8, or 0.9.

19. The method of any of the preceding claims, which further comprises, after administration of the disrupting agent, obtaining a value for (e.g., measuring) the IntIndᵢ as measured by Formula 2 of the genomic complex (e.g., ASMC).

20. The method of claim 19, which further comprises, responsive to the value for the IntIndᵢ as measured by Formula 2, administering one or more additional doses of the disrupting agent to the mammalian subject, or administering one or more different therapies.

21. The method of claim 20, which comprises administering the one or more additional doses of the disrupting agent to the mammalian subject until the IntIndᵢ as measured by Formula 2 in a cell of the subject, is less than 0.9, 0.8, 0.7, 0.6, 0.5, 0.4, 0.3, 0.2, or 0.1.

22. The method of the preceding claims, which further comprises, after administration of the disrupting agent, determining obtaining a value for (e.g., measuring) expression of a gene associated with (e.g., situated at least partially within) the genomic complex (e.g., ASMC).

23. The method of claim 22, which further comprises, responsive to the value for the expression of the gene, administering one or more additional doses of the disrupting agent to the mammalian subject, or administering one or more different therapies.

24. The method of any of the preceding claims, wherein the genomic complex (e.g., ASMC) comprises a gene, an anchor sequence, or two anchor sequences listed in Table 4 or 5.

25. The method of any of the preceding claims, wherein the genomic complex (e.g., ASMC) is bound by a polypeptide selected from CTCF, cohesin, YY1, USF1, TAF3, or ZNF143.

26. The method of any of the preceding claims wherein the genomic complex (e.g., ASMC) is a type 1 or type 2 ASMC.

27. The method of any of the preceding claims wherein disruption of the genomic complex (e.g., ASMC) results in upregulation of expression of a gene situated at least partly within the genomic complex (e.g., ASMC).

28. The method of any of claims 1-26, wherein disruption of the genomic complex (e.g., ASMC) results in downregulation of expression of a gene situated at least partly within the genomic complex (e.g., ASMC).

29. The method of claim 28, wherein the IntIndᵢ as measured by Formula 2 of the ASMC in the cell is at least 0.5, 0.55, 0.6, 0.65, 0.7, 0.75, 0.8, 0.85, or 0.9.
